(19) United States
(12) Patent Application Publication
(10) Pub. No.: US 2016/0215331 A1
(43) Pub. Date: Jul. 28, 2016

(54) FLEXIBLE AND SCALABLE GENOTYPING-BY-SEQUENCING METHODS FOR POPULATION STUDIES

(71) Applicant: Yale University, New Haven, CT (US)

(72) Inventors: Stephen Dellaporta, Branford, CT (US); Christopher Fragoso, Newtown, CT (US); Christopher Heffelfinger, Columbus, OH (US); Maria Morena, Branford, CT (US)

(21) Appl. No.: 14/860,174

(22) Filed: Sep. 21, 2015

Related U.S. Application Data
(60) Provisional application No. 62/107,691, filed on Jan. 26, 2015.

(51) Int. Cl. C12Q 1/68

(52) U.S. Cl. CPC C12Q 1/6874; C12Q 1/6806

(57) ABSTRACT

Flexible and scale methods of genotyping-by sequencing are provided. Typically the methods include the steps of cutting genomic DNA into blunt-ended fragments using a blunt-cutting restriction endonuclease enzyme, dA tailing the fragments and ligating the dA tailed fragments to universal sequencing adapters, enrichment of desired DNA by size-selecting, barcoding and sequencing the size-selected fragments of the genomic DNA. A standard DNA size selection step can be used to capture a small portion of the genome that will be consistent between samples from the same species. The methods improve the efficiency, coverage, data quality and cost over existing methods for reduced representation sequences of genomes.

### CROSS-REFERENCE TO RELATED APPLICATIONS

This application claims benefit of U.S. Provisional Application No. 62/107,691, filed Jan. 26, 2015, the contents of which is incorporated by reference herein in its entirety.

### STATEMENT REGARDING FEDERALLY SPONSORED RESEARCH OR DEVELOPMENT

This invention was made with government support under Grant No. 9420201-15-0001 awarded by National Science Foundation. The U.S. government has certain rights in the invention.

### REFERENCE TO SEQUENCE LISTING

The Sequence Listing submitted as a text file named “YU_6638_ST25.txt,” created on Sep. 14, 2015, and having a size of 7,000 bytes is hereby incorporated by reference pursuant to 37 C.F.R. §1.52(e)(5).

### FIELD OF THE INVENTION

The invention is generally related to methods for reduced representation genotyping by sequencing that employ a mixture or library of blunt-ended genomic DNA fragments, and preferably for use with universal sequencing adapters for population-based genomic sequencing.

### BACKGROUND OF THE INVENTION

Genome re-sequencing has emerged as the principal means for identifying both the genotypes of single individuals and genetic variation within populations or species. Methods such as whole genome and whole exome sequencing can generate data on large numbers of common and rare variants and discover previously uncharacterized variants. Further, population genomics via sequencing shows reduced ascertaimnent bias relative to microarrays and other a posteriori methods.

Improvements in sequencing chemistry, methodologies, hardware, and software have increased sequencing read quantity and length, improved multiplexing scalability, and added further robustness to genotyping calls (Lorean N J, et al, Nature biotechnology 30(5):434-439 (2012); Lam, et al., Nature biotechnology 30(1):78-82 (2012)).

Associated bioinformatics have seen similar advancement in the filtering of false positives, imputation of missing data, and utilization of datasets for genomics (Sousa, et al, Nature reviews Genetics, 14(6):404-414 (2013); Nekrutenko, et al, Nature reviews Genetics, 13(9):667-672(2012); Abecasis, et al., Nature, 467(7319):1061-1073 (2010); Nielsen, et al., Nature reviews Genetics, 12(6):443-451(2011); Ruffalo, et al., Bioinformatics, 27(20):2790-2796(2011); Li, et al., Briefings in bioinformatics, 11(5):473-483(2010)).

In the course of these advances, two major avenues for genome re-sequencing have emerged: whole genome sequencing (WGS) and a variety of methods collectively referred to as reduced representation sequencing (RRS).

WGS methodologies attempt to query the entire genome in an as unbiased a manner as technically possible by constructing and sequencing libraries of randomly sheared genomic DNA. Millions of short reads are aligned to a reference genome to identify variants. While per-base error rate in most NGS methodologies is low, technical limitations, insufficient sequencing depth, and sequence and structural inaccuracies in the reference genomes can result in numerous errors (Nielsen, et al., Nature reviews Genetics, 12(6):443-451(2011)).

Deep sequence coverage of overlapping reads can significantly reduce errors in variant calling. Hence, each position in the genome is represented by many overlapping reads on both strands of DNA that result in highly robust genotype calls and reduced errors from PCR, sequencing artifacts, and alignment errors. The amount of sequencing required to achieve high coverage, especially in large eukaryotic genomes such as many plants, can be prohibitively expensive. This restricts the application of high-coverage WGS-based genotyping. Therefore, WGS methods that rely on 20× to 30× coverage are preferred when attempting to identify sample specific variation or very limited numbers of samples in a population are available.

Low-coverage (LC) WGS is typically kept around 5× and, in some cases, less than 1× mean coverage per base for a given sample. LC-WGS reduces the cost and improves the ability to multiplex samples in a single sequencing run. Its limitation is the accuracy of variant calling due to incomplete genome coverage and the inability to distinguish variants and inherent errors. For instance, polymorphisms may be lost in a sample due to low coverage or subsequent filtering during computational steps. Errors introduced by PCR and sequencing may be misidentified as variants when coverage is low. Nevertheless, when a reference genome and sufficient samples are available to infer haplotype structure, statistical methods such as imputation may result in variant calling that rivals that produced by HCWGS both in terms of quantity and accuracy for a fraction of the cost (Nielsen, et al., Nature reviews Genetics, 12(6):443-451(2011); Li, et al., Genome research, 21(6):940-951(2011); Abecasis, et al., Nature, 491(7422):56-65(2012); Wu, et al., BMC genomics, 11:469 (2010)). Yet, without some form of cross-sample validation of variation, LC-WGS is at a disadvantage to high coverage sequencing.

A second category of genome re-sequencing can be collectively called reduced representation sequencing (RRS) methodologies. Quite simply, RRS methodologies reduce a genome's complexity by enriching, separating, or eliminating a portion of the genome prior to sequencing. Some methods attempt to increase the informative fraction of the sequenced genome, such as exome sequencing (Bamshad, et al., Nature reviews Genetics, 12(11):745-755 (2011); Ng, et al., Nature, 461(7261):272-276 (2009)), while others ensure a consistent portion of the genome is retargeted for sequencing among samples (Wu, et al., BMC genomics, 11:469 (2010); Turner et al., Annual review of genomics and human genetics, 10:263-284 (2009); Elshire et al., PloS one, 6(5):e19379(2011); Miller, et al., Genome research, 17(2):240-248(2007); Van Tassell et al., Nature methods, 5(3):247-252(2008); Greminger, et al., BMC genomics, 15:16(2014); Poland, et al., Plant Genome-Us, 5 (3): 92-102 (2012)).

Exome sequencing, the most common RRS methodology, is based on oligonucleotide capture technologies, where short DNA fragments bind complementary targets of interest. Captured fragments are then isolated from the rest of the genome and sequenced. Large oligo capture arrays allow high specificity even when interrogating large genomic regions, such as the human exome. While this technology can be applied to almost any set of targets, initial implementation can be very costly and requires the genome of interest be well characterized. Alternative RRS technologies are restriction-site associated DNA (RAD) sequencing (Miller, et al., Genome research, 17(2):240-248(2007)) spin-off methods called double-digest RAD or ddRAD (Peterson et al., PloS one, 7(5):e37135 (2012); 2b-RAD (Wang, et al., Nature methods, 9(8):808-810 (2012)), and a related method called Genotyping-By-Sequencing or GBS (Elshire, et al., PloS one, 6(5):e19379(2011); U.S. Pat. Nos. 8,685,889; 8,785,353; 9,023,768 and U.S. application Ser. No. 14/626,822 to Keygene N.V.). These methods rely on an initial digest of sample DNA by restriction enzyme to reduce genome representation. The 2b-RAD method uses a Type IIb restriction enzyme, which cuts at two points to produce a fixed-size dsDNA fragment. In ddRAD, a second digest of genomic DNA (gDNA) by a different enzyme follows the first. In both RAD and ddRAD, a biotinylated adaptor specific to the initial enzyme captures DNA fragments of interest (Miller, et al., Genome research, 17(2):240-248(2007)). 2b-RAD uses size selection to capture fragments of interest. RAD technologies and GBS can be adapted to poorly characterized genomes, but lack the specificity to regions of interest of exome sequencing. In addition, much of the sequence will originate from non-informative, repetitive regions.

GBS is similar to RAD sequencing whereby a restriction enzyme digest of genomic DNA produces a size spectrum of DNA fragments. As restriction enzyme sites are reasonably fixed (barring polymorphism) within a species' genome, homologous regions will produce size spectrums that are consistent between members of a population. Reduced representation is achieved by sequencing a small range of fragment sizes, rather than by capture of biotinylated adaptor. GBS can target as little as 2.3% of a genome for sequencing (Elshire, et al., PloS one, 6(5):e19379 (2011). More importantly, this small portion remains sufficiently consistent across samples to produce comparative results even in highly diverse species (Romay, et al., Genome biology, 14(6):R55 (2013)), especially when other resources, such as NAM lines or a high quality reference genome, are available to guide calls. In maize, which has undergone extensive GBS-based research, there is approximately tenfold more inter-accession diversity than exists across the spectrum of human populations (Chia, et al., Nature genetics, 44(7):803-807 (2012); Tenaillon, et al., Proc Natl Acad Sci USA, 98(16):9161-9166 (2001)).

This methodology is easily implemented, low cost, adaptable to poorly characterized genomes, and suitable for large-scale multiplexing of both library preparation and sequencing (Elshire et al., PloS one, 6(5):e19379 (2011)). Interest in the GBS protocol has resulted in expansions to the original protocol and improved computational data filtering and imputation (Sonah, et al., PloS one, 8(1):e54603 (2013); Glaubitz, et al., PloS one, 9(2):e90346 (2014); Poland, et al. PloS one, 7(2):e32253 (2012); Spindel, et al., Theoretische and angewandte Genetik, 126(11):2699-2716 (2013)).

In spite of its popularity, several issues limit the adoption of GBS methodology. One key issue is the requirement of customized barcoded adaptors specific to a single restriction overhang sequence. This greatly reduces flexibility and increases the cost of implementation.

Efficient next-generation sequencing of large numbers of samples often requires methodologies that capture a reduced but consistent portion of the genome between samples. These methodologies are collectively referred to as reduced representation sequencing.

The standard methods for performing reduced representation sequencing in plants involve the use of restriction enzymes to interrogate the genome at fixed points.

Many areas critical to agricultural production and research, such as the breeding and trait mapping in plants and livestock, require robust and scalable genotyping platforms. Genotyping-by-sequencing (GBS) is a one such method highly suited to non-human organisms. In the GBS protocol, genomic DNA is fractionated via restriction digest, then reduced representation is achieved through size selection. Since many restriction sites are conserved across a species, the sequenced portion of the genome is highly consistent within a population. Thus, the GBS protocol is highly suited for experiments that require surveying large numbers of markers within a population, such as those involving genetic mapping, breeding, and population genomics.

Next generation sequencing has clearly demonstrated its utility for generating large, robust datasets for population genomics in humans. Migrating these methods and utilities to other reference organisms has been met with difficulty, however. The major obstacle has traditionally been poor or non-existent reference genomes combined with the high cost of developing oligo capture arrays required for exome sequencing, the most popular method for genotyping in humans. Nonetheless, low-cost, highly scalable sequencing is a critical requirement for large-scale population genomics in any species.

While effective in a wide variety of species, these methods are often costly, inflexible, and produce large amounts of noise in the data.

Therefore, it is an object of the invention to provide sensitive and efficient methods for genotyping-by-sequencing that overcome the requirement for customized barcoded adaptors specific to a single restriction overhang sequence.

It is a further object of the invention to provide robust, reliable and reproducible methods for enrichment, sequencing and identification of corresponding genomic nucleic acid sequence domains from a multiplicity of samples simultaneously.

### BRIEF SUMMARY OF THE INVENTION

Methods and compositions for blunt-cutting restriction enzyme-based reduced representation sequencing are provided. The methods utilize one or more restriction endonuclease enzymes that produce blunt-ended DNA fragments, amenable to ligation with universal adaptors. The methods are compatible with any blunt-ended restriction enzymes, providing flexibility in selection of the size, genic coverage, position, etc. of digestion products obtained from any given genome. The methods can also include a dual indexing system that enables exceptional multiplexing of individual samples, and a simple bead-based library preparation protocol that allows in-solution reaction cleanup and size selection in microliter plates. The methods provide a scalable and flexible means for analysis of DNA sequence patterns, such as polymorphisms, amongst a large pool of genomes. The methods reduce costs required to initially implement genotypic-by-sequencing, and provide a rapid and effective means to switch enzymes, enabling changes in experimental design to better meet experimental needs.

The methods include bringing into contact a first nucleic acid sample with one or more blunt-cutting restriction endonuclease enzyme(s) to form a reaction mix; incubating the reaction mix under conditions that allow the restriction endonuclease enzyme(s) to cut the nucleic acid to produce a digested nucleic acid sample containing blunt-ended nucleic acid fragments; ligating the blunt-end nucleic acid fragments in the digested nucleic acid sample to universal sequencing adapters to produce an adapter-ligated digested nucleic acid sample; performing a complexity reduction on the blunt-ended nucleic acid digestion fragments from the adapter-ligated digested nucleic acid sample to form an enriched nucleic acid sample; labelling the nucleic acid digestion fragments in the enriched nucleic acid sample with one or more labels to form a labelled, enriched nucleic acid sample; and sequencing at least a portion of the enriched nucleic acid sample to obtain a first sequence. An exemplary nucleic acid sample is genomic DNA, preferably whole genomic DNA. Genomic DNA samples can include genomic DNA from one or more than one individual. In some embodiments, the sequencing is paired-end sequencing.

In some embodiments, labelling the nucleic acid digestion fragments with one or more labels to form a labelled, enriched nucleic acid sample can be carried out without performing a complexity reduction on the blunt-ended nucleic acid digestion fragments from the adapter-ligated digested nucleic acid sample. Therefore, in some embodiments, performing a complexity reduction is optional.

When the methods do not include complexity reduction, the blunt-end nucleic acid fragments ligated to universal sequencing adapters are labelled with one or more labels to form a labelled, enriched nucleic acid sample, and sequenced to obtain a first sequence.

The methods can be consecutively or simultaneously performed on a second, third, or more nucleic acid sample(s) to obtain a second, third or more labelled, enriched nucleic acid sample(s). The multiplicity of labelled, enriched nucleic acid samples can be combined prior to sequencing. The methods can optionally include aligning the multiplicity of sequences and determining one or more polymorphisms between the multiplicity of nucleic acid samples in the alignment. In some embodiments, ligating the blunt-end DNA fragments to universal sequencing adapters includes the step of adding adenine to the 3′ end of the DNA fragments. Exemplary universal sequencing adapters are ILLUMINA® Y-adapters.

Typically, some or all of the methods are carried out with the nucleic acid samples bound to a solid phase. For example, the nucleic acid samples can be bound to a solid phase following digestion with blunt-ended restriction enzymes. An exemplary solid phase is a multiplicity of magnetic beads, such as SPRI beads. When nucleic acids are bound to a solid phase, the methods can include one or more steps of washing the nucleic acids-bound to the solid phase. Preferably, the methods are carried out within the same well of a microtiter plate.

In some embodiments, the methods can optionally include complexity reduction. Typically, complexity reduction includes fractionation of the adapter-ligated digested nucleic acid sample on the basis of size. For example, fractionation of the adapter-ligated digested nucleic acid sample on the basis of size can be carried out by selective hybridization of adapter-ligated digested nucleic acids to magnetic beads. The enriched nucleic acid sample typically includes DNA fragments with an average length of between 200 and 800 base pairs, inclusive. In some embodiments, the methods do not include complexity reduction.

Typically, the DNA samples are labelled following adapter ligation and enrichment by introducing one or more sequence identifiers to each DNA fragment by polymerase chain reaction, preferably using between 2 and 10 cycles, most preferably 5 or 6 cycles. The oligonucleotide primers used in the polymerase chain reaction can independently include a sequence identifier of between four and ten nucleic acid residues, inclusive. In some embodiments, two unique sequence identifiers are added to each nucleic acid fragment. One or more of the restriction enzymes can be selected based on in silico modeling to create a virtual restriction enzyme digest for the genomic nucleic acid sample.

Kits for including reagents necessary to conduct the methods are also provided. Typically, the kits include one or more blunt-cutting restriction endonuclease enzyme(s); universal sequencing adapters; SPRI beads; and instructions for carrying out the methods. In some embodiments, the kit also includes oligonucleotide primers each comprising unique nucleotide sequences of between four and ten nucleic acid residues for labelling the enriched nucleic acid sample.

In some embodiments, the first nucleic acid sample has high sequence complexity. The first nucleic acid sample can include double stranded DNA. The first nucleic acid sample can include genomic DNA. In some embodiments, the enriched nucleic acid fragments have an average length of between approximately 200 and 800 base pairs. The methods can enrich a small proportion of the first nucleic acid sample. For example, when the first nucleic acid sample includes genomic DNA, the methods can enrich between about 0.1% and 5% of the genomic DNA, for example 5%, 4%, 3%, or less than 3% of the genomic DNA. In a certain embodiment, the methods enrich approximately 2.3% of the genomic DNA.

### BRIEF DESCRIPTION OF THE DRAWINGS

FIGS. 1A-1B are histograms showing fraction of reads (0.0-1.0) with mapping quality of ≧30 and ≧20, for each of unpaired reads (white) and paired reads (black), respectively for the maize (FIG. 1A) and rice (FIG. 1B) genomes, respectively.

FIGS. 2A-2B are histograms showing fraction of reads (0.0-1.0) with mapping quality of ≧30 and ≧20, for each of unpaired reads (grey) and paired reads (black), for each of MlyI; AluI; RsaI; DRaI; EcoRV; StuI; HaeIII; and HincII, respectively. The relative position of WG MQ≧30 and ≧20 is indicated by black and grey horizontal lines, respectively.

FIGS. 3A-3F are histograms. FIG. 3A shows the site count (0-2,500,000) for each of AluI; HaeIII; MlyI; RsaI; DRaI; HincII; EcoRV; and StuI, respectively. FIG. 3B is an enlarged view of the region of FIG. 3A corresponding to the site count from 0-160,000 for each of DRaI; HincII; EcoRV; and StuI, respectively. FIG. 3C shows the site count (0-900,000) for each of AluI; HaeIII; MlyI; RsaI; DRaI; HincII; EcoRV; and StuI, respectively. FIG. 3D is an enlarged view of the region of FIG. 3C corresponding to the site count from 0-120,000 for each of DRaI; HincII; EcoRV; and StuI, respectively. FIG. 3E shows mean reads per site (0-25) for each of AluI; HaeIII; MlyI; RsaI; DRaI; HincII; EcoRV; and StuI, respectively. FIG. 3F shows mean reads per site site (0-80) for each of AluI; HaeIII; MlyI; RsaI; DRaI; HincII; EcoRV; and StuI, respectively. FIG. 3G is a schematic showing the relative sizes of nucleic acid fragments generated in a nucleic acid sequence containing three restriction endonuclease cleavage sequences (represented by “R”) and the corresponding nucleic acid fragments generated by cleavage at predicted sites; mis-paired sites; singlets and null-cleavage, respectively.

FIGS. 4A-4B are graphs showing distance (base pairs) for each of restriction endonuclease enzymes MlyI; RsaI; DRaI; EcoRV; StuI; HaeIII; and HincII, respectively, for each of the maize (FIG. 4A) and rice (FIG. 4B) samples, respectively.

FIGS. 5A-5B are histograms showing the fraction of sites in or overlapping genes in all sites (white) and covered sites (black) for each of MlyI; AluI; RsaI; DRaI; EcoRV; StuI; HaeIII; and HincII, respectively, for each of the maize (FIG. 5A) and rice (FIG. 5B) samples, respectively. The intronic/exonic fraction of genome is indicated by a horizontal line.

FIGS. 6A and 6B are histograms showing marker count for samples with post-filter marker call for fragments produced by RsaI (FIG. 6A); and HincII (FIG. 6B), respectively.

FIGS. 7A-7D are graphs showing log 10(p) (1-15) for each chromosome (1-10), showing data for the RsaI GBS dataset su1 map (FIG. 7A); RsaI GBS dataset y1 map (FIG. 7B); HincII GBS dataset su1 map (FIG. 7C); and HincII GBS dataset y1 map (FIG. 7D), respectively.

FIGS. 8A-8B are graphs showing fraction shared with original sample (0.0-1.0) for paired-end reads for each of post-imputation genomewide basecalls (♦); and post-filter markers (□), respectively, for sub-samplings of RsaI F₂-44 (FIG. 8A) and HincII F₂-23 (FIG. 8B).

FIGS. 9A-9B are line graphs showing fraction of sites covered over Guanine/Cytosine fraction for predicted fragments and reads aligning to predicted fragments for each of MlyI; AluI; RsaI; DRaI; EcoRV; StuI; HaeIII; and HincII, respectively, for each of the maize (FIG. 9A) and rice (FIG. 9B) samples, respectively.

FIGS. 10A-10H are graphs showing the difference in base fraction between covered and predicted sites in genic regions (FIG. 10A) and non-genic regions (FIG. 10B) for each of MlyI; AluI; RsaI; and DRaI in maize; predicted sites in genic regions (FIG. 10C) and non-genie regions (FIG. 10D) for each of; EcoRV; StuI; HaeIII; and HincII in maize; predicted sites in genic regions (FIG. 10E) and non-genic regions (FIG. 10F) for each of for each of MlyI; AluI; RsaI; and DRaI in rice; and predicted sites in genic regions (FIG. 10G) and non-genic regions (FIG. 10H) for each of EcoRV; StuI; HaeIII; and HincII in rice, respectively. Changes between predicted and covered sites are plotted one (⋄) to two (∘) bases upstream of enzyme recognition motif and guanine one to two bases downstream for each sample maize and rice. Mean value (□) is also shown for 1-12 bp upstream or downstream. Error bars represent two standard deviations based on nucleotide ratios three through twelve bases upstream and downstream.

FIG. 11 is a schematic diagram demonstrating how restriction endonuclease enzymes enable consistent representation of a limited set of sites across multiple samples within a given population.

FIG. 12 is a flow chart showing an exemplary GBS method as disclosed herein.

### DETAILED DESCRIPTION OF THE INVENTION

### I. Definitions

As used herein, “enrich” and “enrichment” refer to an increase in the proportion of a component relative to other components present or originally present. In the context of nucleic acids, enrichment of nucleic acids in a sample refers to an increase in the proportion of the nucleic acids in the sample relative to other molecules in the sample. “Selective enrichment” is enrichment of particular components relative to other components of the same type. In the context of nucleic acid fragments, selective enrichment of a particular nucleic acid fragment refers to an increase in the proportion of the particular nucleic acid fragment in a sample relative to other nucleic acid fragments present or originally present in the sample. The measure of enrichment can be referred to in different ways. For example, enrichment can be stated as the percentage of all of the components that is made up by the enriched component. For example, particular nucleic acid fragments can be enriched in an enriched nucleic acid sample to at least 90% of the enriched nucleic acid sample.

As used herein, “nucleic acid fragment” refers to a portion of a larger nucleic acid molecule. A “contiguous nucleic acid fragment” refers to a nucleic acid fragment that represents a single, continuous, contiguous sequence of the larger nucleic acid molecule. A “naturally occurring nucleic acid fragment” refers to a nucleic acid fragment that represents a single, continuous, contiguous sequence of a naturally occurring nucleic acid sequence. “DNA fragment” refers to a portion of a larger DNA molecule. A “contiguous DNA fragment” refers to a DNA fragment that represents a single, continuous, contiguous sequence of the larger DNA molecule. A “naturally occurring DNA fragment” refers to a DNA fragment that represents a single, continuous, contiguous sequence of a naturally occurring DNA sequence.

As used herein, “naturally occurring” refers to a molecule that has the same structure or sequence as the corresponding molecule as it exists in nature. A naturally occurring molecule or sequence can still be considered naturally occurring when it is coupled to or incorporated into another molecule or sequence.

As used herein, “nucleic acid sample” refers to a composition, such as a solution, that contains or is suspected of containing nucleic acid molecules. An “enriched nucleic acid sample” is a nucleic acid sample in which nucleic acids, particular nucleic acid fragments, or a combination thereof, are enriched. “DNA sample” refers to a composition, such as a solution, that contains or is suspected of containing DNA molecules. An “enriched DNA sample” is a DNA sample in which DNA, particular DNA fragments, or a combination thereof, are enriched.

As used herein, “whole genomic DNA” refers to all of an organism's chromosomal DNA as well as DNA contained in the mitochondria and, for plants, in the chloroplast. Whole genomic DNA for a given organism includes the sum total of all genetic material for that organism. A “genomic DNA sample” can contain entire genomes of any size and complexity. A “Genomic library” is a collection of clones made from a set of randomly generated overlapping DNA fragments that represent the entire genome of an organism.

As used herein, the term “nucleotide” refers to a molecule that contains a base moiety, a sugar moiety and a phosphate moiety. Nucleotides can be linked together through their phosphate moieties and sugar moieties creating an inter-nucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. A non-limiting example of a nucleotide would be 3′-AMP (3′-adenosine monophosphate) or 5′-GMP (5′-guanosine monophosphate). There are many varieties of these types of molecules available in the art and available herein.

As used herein, the terms “oligonucleotide” or a “polynucleotide” are synthetic or isolated nucleic acid polymers including a plurality of nucleotide subunits.

As used herein, the term “Restriction endonuclease” or “Restriction enzyme” or “RE enzyme” is any enzyme that recognizes one or more specific nucleotide target sequences within a DNA strand, to cut both strands of the DNA molecule at or near the target site. A “blunt-cutting” Restriction endonuclease, or “blunt RE” is any Restriction endonuclease enzyme that cuts both strands of the DNA at the same nucleotide bond and does not produce a single-strand overhang.

As used herein the terms “universal” or “standard” adapters are used interchangeably to refer to sequencing adapters that are not customized for use with a specific restriction endonuclease enzyme. Universal adapters can be ligated to double-stranded DNA fragments produced by restriction endonuclease activity from more than a single enzyme. DNA fragments having a common terminal or “end”, such as the blunt ended fragments produced by digestion with one or more blunt-cutting restriction enzymes can be ligated to universal sequence adapters can at one or both ends. Universal sequencing adapters can also bind to blunt-ended DNA fragments having a common single base overhang, such as a dN nucleotide, where N is any of adenine, thymine, uracil or guanine.

As used herein, the term “Complexity reduction” refers to the step of reducing the complexity of a nucleic acid sample, for example, by the generation of a subset of the same sample. Reducing the complexity of a nucleic acid sample produces a sub-population that is enriched for nucleic acids having desired sequences or lacking nucleic acids having undesirable sequences. Complexity reducing methods typically employ sequence-specific hybridization to capture nucleic acids from a source such as genomic DNA, DNA libraries or RNA. The resulting “enriched” subset can be used as probes, or can be quantitated or sequenced. This subset can be representative of the whole (i.e. complex) sample and can be a reproducible subset. For example, a reproducible subset is one in which when the same or similar samples are reduced in complexity using the same or similar methods, the same, or at least a comparable, subset is obtained.

As used herein, the term “Identity,” as known in the art, is a relationship between two or more nucleic acid sequences, as determined by comparing the sequences. In the art, “identity” also means the degree of sequence relatedness between nucleic acid sequences as determined by the match between strings of such sequences. “Identity” can also mean the degree of sequence relatedness of a nucleic acid sequence as compared to the full-length of a reference nucleic acid sequence. “Identity” and “similarity” can be readily calculated by known methods, including, but not limited to, those described in (Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). In general, variants of oligonucleotides, nucleic acid sequences, nucleotide analogs, or nucleotide substitutes thereof and proteins described herein typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent identity to the stated sequence or the native sequence. Those of skill in the art readily understand how to determine the identity of two or more nucleic acid sequences, such as genes. Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). By way of example, nucleic acid sequences may be identical to the reference sequence, that is 100% identical, or may include up to a certain integer number of nucleotide alterations as compared to the reference sequence such that the % identity is less than 100%. Such alterations are selected from: at least one nucleotide substitution, deletion, or insertion and wherein said alterations may occur at the terminal positions of the reference sequence or anywhere between those terminal positions, interspersed either individually among the nucleotides in the reference sequence or in one or more contiguous groups within the reference sequence. The number of nucleotide alterations for a given % identity is determined by multiplying the total number of nucleic acids in the reference sequence by the numerical percent of the respective percent identity (divided by 100) and then subtracting that product from said total number of nucleic acids in the reference sequence.

As used herein, the term “polymorphism” means variations of a nucleotide sequence in a population. For example, polymorphism can be one or more base changes, an insertion, a repeat, or a deletion. Polymorphisms can be single nucleotide polymorphisms (SNP), or simple sequence repeat (SSR). SNPs are variations at a single nucleotide, e.g., when an adenine (A), thymine (T), cytosine (C) or guanine (G) is altered. Generally a variation must generally occur in at least 1% of the population to be considered a SNP.

As used herein, “Isolated,” “isolating,” “purified,” “purifying,” “enriched,” and “enriching,” when used with respect to nucleic acids of interest (e.g., DNA such as intact or fragmented genomic DNA, etc.,), indicate that the nucleic acids of interest at some point in time were separated, enriched, sorted, etc., from or with respect to other cellular material to yield a higher proportion of the nucleic acids of interest compared to the other cellular material, contaminates, or active agents such as enzymes, proteins, detergent, cations or anions. “Highly purified,” “highly enriched,” and “highly isolated,” when used with respect to nucleic acids of interest, indicates that the nucleic acids of interest are at least about 70%, about 75%, about 80%, about 85%, about 90% or more, about 95%, about 99% or 99.9% or more purified or isolated from other cellular materials, contaminates, or active agents such as enzymes, proteins, detergent, cations or anions. “Substantially isolated,” “substantially purified,” and “substantially enriched,” when used with respect to nucleic acids of interest, indicate that the nucleic acids of interest are at least about 70%, about 75%, or about 80%, more usually at least 85% or 90%, and sometimes at least 95% or more, for example, 95%, 96%, and up to 100% purified or isolated from other cellular materials, contaminates, or active agents such as enzymes, proteins, detergent, cations or anions.

As used herein, the term “Ligating” refers to enzymatic reactions in which two double-stranded DNA molecules are covalently joined, for example, catalyzed by a ligase enzyme.

As used herein, the terms “Aligning” and “Alignment” refer to the comparison of two or more nucleotide sequence based on the presence of short or long stretches of identical or similar nucleotides. Several methods for alignment of nucleotide sequences are known in the art, as will be further explained below.

As used herein, the term “subject” includes, but is not limited to, animals, plants, bacteria, viruses, parasites and any other organism or entity. The subject can be a plant. The subject can be an animal, such as a vertebrate, more specifically a mammal (e.g., a human, horse, pig, rabbit, dog, sheep, goat, non-human primate, cow, cat, guinea pig or rodent), a fish, a bird or a reptile or an amphibian. The subject can be an invertebrate, more specifically an arthropod (e.g., insects and crustaceans). The term does not denote a particular age or sex. Thus, adult and newborn subjects, as well as fetuses, whether male or female, are intended to be covered. A patient refers to a subject afflicted with a disease or disorder. The term “patient” includes human and veterinary subjects. A cell can be in vitro. Alternatively, a cell can be in vivo and can be found in a subject. A “cell” can be a cell from any organism including, but not limited to, a bacterium.

### II. Methods

Genotyping by sequencing methods amenable to targeting and enriching specific nucleic acid fragments from a mixture of genomic nucleic acids using one or more blunt-cutting sequence-specific restriction endonuclease enzymes are provided. The methods facilitate and enhance reduced representation sequencing, and can be used in applications ranging from genotyping a specific individual to preparing genetic variation profiles amongst populations of species. The disclosed approaches, which are compatible with all blunt-end restriction enzymes, are high-throughput, scalable to large sample sizes, and have a lower cost to implement than other methods.

The methods typically include digestion of genomic DNA with one or more “blunt-cutting” restriction endonucleases (RE) enzymes (i.e., restriction endonucleases that do not result in a single-strand overhang at the cut site) that cleave between contiguous nucleotide bases to leave a phosphate group at the 5′ end of the resulting DNA strand. The methods can be carried out using a range of RE enzymes having different recognition motifs to provide sequencing information for a range of different genomic DNA fragments without the need for customized sequencing adapters. Therefore, the methods are compatible with standard universal sequencing adapters without the need for end-repair or “polishing” of genomic DNA fragments with sticky-ends. For example, the methods are compatible with ILLUMINA® library preparation protocols.

Adapter-ligated samples can be subjected to an optional complexity reduction, for example, a size-selection process for nucleic acid fragments between 200 and 800 nucleotide base-pairs (bp) in length. The selection step enriches only the genomic DNA fragments that were generated by restriction sites separated by between 200 and 800 bp apart from the remaining (majority) of the genomic DNA. The enriched, isolated samples can be labelled, e.g., by bar-coding, to be directly compatible with a dual-indexed barcoding system identification when using ILLUMINA® Y-adapters for sequencing. For example, a low-cycle PCR reaction can add short barcoding sequences to the ends of each DNA strand. The combination of two barcodes is unique to each sample, and sequence information can be reassigned to each individual sample following sequence acquisition. Barcoded samples can be pooled and sequenced according to any sequencing protocol.

Blunt-end GBS methods using combinations of commercially-available blunt RE enzymes can correctly map several individuals from a population and are readily adaptable to different genomes and highly amenable to multiplexing. Therefore, methods for multiplexing of individual samples are provided. For example, the methods enable high-throughput sequencing of targeted regions of a pool of genomic DNA for the selective sequencing of exons or sets of protein-coding genes implicated in specific diseases; whole human exome sequencing (for example, in cancer or disease cohorts) (Poland, Plant Genome-Us, 5(3):92-102 (2012); Peterson, et al., PloS one, 7(5):e37135 (2012); Wang, et al. Nature methods, 9(8):808-810 (2012)) and re-sequencing of specific regions as a follow-up to genome-wide association studies.

The methods can be used to identify and genotype informative markers in a single step. High-throughput methods that utilize blunt-cutting RE enzymes to accelerate the identification of polymorphisms for efficient and reliable genotyping of a pool of samples are provided. Specifically, the methods can be used for any purpose where performing a complexity reduction assists in the identification of a large number of sequences. In some embodiments the methods can identify one or more single nucleotide polymorphisms (SNPs) in two or more different samples. For example, the methods can be used to produce one or more libraries of size-selected genomic fragments. The libraries can be sequenced and aligned to identify or confirm single nucleotide polymorphisms at a multiplicity of locations amongst a multiplicity of samples.

The methods can include immobilizing the digested nucleic acid fragments to Solid Phase Reversible Immobilization (SPRI) beads of DNA fragments using microtiter plates. Therefore, the methods enable selection of specific nucleic acid fragments based on size using without the need for gels or columns.

When the methods include complexity reduction, the methods can enrich a small proportion of the first nucleic acid sample. For example, when the first nucleic acid sample includes genomic DNA, the methods can enrich from about 0.1% to about 5% of the genomic DNA, for example about 5%, 4%, 3%, or less than 3% of the genomic DNA. In a certain embodiment, the methods enrich approximately 2.3% of the genomic DNA.

Preferably, the methods produce data that is of equal quality or of better quality than data obtained using other genotyping by sequencing methods that do not employ restriction endonuclease enzymes that produce blunt DNA fragments.

Because the methods enable use of the same oligonucleotide adapters with any blunt-end restrictions endonuclease enzymes, the methods impart flexibility in the portion of the genome that will be represented in the final dataset. Further, methods including restriction enzymes that do not produce overhangs enable repeated application with the same pool of sequencing adapters. Therefore, a common set of sequencing reagents can be paired with the restriction enzymes necessary to produce nucleic acid fragments having the desired length/including the desired sequence. Data quality can be assessed by a metric such as sequence mapping quality value (MQ) for sequencing reads.

The methods described herein include one or more of the following steps: (a) bringing into contact one or more blunt-cutting restriction endonuclease enzymes with a first nucleic acid sample in an appropriate buffer to form a reaction mix; (b) incubating the reaction mix under conditions that allow cutting of the nucleic acid by the RE enzyme(s) to yield a pool of blunt DNA fragments; (c) incorporating a non-templated dAMP on the 3′ end of the blunt DNA fragment; (d) binding DNA fragments to adapters; (c) optional complexity reduction; (d) labelling the optionally size-selected DNA fragments; and (e) pooling and sequencing the labelled nucleic acid sample. Each of these steps is discussed in more detail, below.

A. Production of Blunt-Ended Genomic DNA Fragments

The disclosed methods typically include producing fragments of genomic DNA having a terminal base pair, as opposed to a “sticky end” single-stranded overhang. Restriction enzymes that generate blunt ended-fragments, rather than ones with staggered ends enable preparation of a genomic DNA fragment library without an end-repair step. The methods are compatible with a broad range of restriction endonuclease enzymes. Restriction enzymes can be selected to produce fragments with a desired average length.

1. Preparation of Nucleic Acid Samples

Any of the methods described herein can include the step of preparing a nucleic acid sample. Methods for preparation of nucleic acid samples are known in the art. For example, methods for collecting various bodily or cellular samples and for extracting nucleic acids are well known in the art. In some embodiments, nucleic acid samples are obtained from cells, tissues, or bodily fluids containing nucleic acid. Exemplary source organisms for nucleic acid samples include bacteria, fungi, animals and plants.

Exemplary bodily samples include, but are not limited to, blood, lymph, urine, gynecological fluids, and biopsies. Bodily fluids can include blood, urine, saliva, or any other bodily secretion or derivative thereof. Blood can include whole blood, plasma, serum, or any derivative of blood. The sample can include cells, particularly eukaryotic cells from swabs and washings or tissue from a biopsy. Samples can be obtained from a subject by a variety of techniques including, for example, by scraping, washing, or swabbing an area, by using a needle to aspirate bodily fluids, or by removing a tissue sample (i.e., biopsy).

If the nucleic acid sample is within cells, tissue or bodily fluids, preparation and purification of the nucleic acid from the sample can include lysis of cells, such as cells within blood. The nucleic acid sample can provided as a lyophilized, dry powder, or in solution, such as an aqueous solution. Preferably, the nucleic acid sample is provided in a solution that forms part of a reaction mix. A reaction mix can include the reagents, e.g., buffers, etc., that enable and enhance the activities of one or more restriction endonuclease enzymes.

In some embodiments, the genomic DNA is bound to a solid phase prior to digestion.

2. Selection of Restriction Endonuclease Enzymes

Methods for producing an enriched pool of nucleic acid samples of a desired size, produced by the choice of appropriate blunt-cutting RE enzymes are provided. The blunt-cutting RE enzyme(s) can be selected to yield a specific nucleic acid fragment (i.e., to include one or more nucleic acid sequence(s) of interest) or to produce a series of different nucleic acid fragments, having known size(s). The selection of appropriate restriction endonuclease (RE) enzymes can influence many factors that contribute to the quality of sequencing data produced by the methods. Therefore, restriction endonuclease (RE) enzymes can be selected to meet the needs of a given experiment by, for example, enriching informative sequences and minimizing repetitive and ambiguous reads. High levels of multiplexing and consistent genome representation can be achieved by utilizing enzymes with complex recognition motifs, while enzymes with simple motifs may better serve experiments requiring extensive variant identification. Genome size, repetitiveness, methylation status, and quality of the associated reference sequence are all factors that may ultimately affect enzyme selection.

As discussed in the Examples below, a panel of restriction endonuclease enzymes was selected for testing on B73 maize and Nipponbare rice genomic DNA. The quality of the data was confirmed by identifying that the vast majority of reads from each enzyme aligned to restriction sites predicted in silico. It has been established that RE enzyme parameters can influence experimental outcome. As described in the Examples, the sequenced portion of the genome is adaptable by selecting RE enzymes based on motif length, complexity, and methylation sensitivity.

Key factors that should be balanced in any GBS experiment include multiplexing, resolution, and coverage. Optimal marker density for QTL mapping and other population genomics increases with the expected number of recombination events per sample and sample size. This can be empirically calculated to a degree (Beissinger, et al., Genetics, 193(4):1073-1081(2013)). All three are directly affected by enzyme choice. The choice of enzyme is therefore highly dependent on available data resources. In a population with a well-established reference genome and little heterozygosity, imputation may reconcile a dataset with large amounts of missing markers into a robust genetic map. In an organism with a contig-level or non-existent reference genome, selecting an enzyme with a sparse profile so each marker is covered in a large number of samples may be desirable.

Preferably, RE are selected to provide optimal sequencing data as desired by the needs of the user.

a. In Silico Site Prediction

Selection of appropriate blunt-cutting restriction endonuclease enzymes for use with the described methods can be aided or enhanced using computational modelling. Therefore, in some embodiments, in silico modelling can be used to predict whether a given restriction endonuclease recognition sequence would be covered by sequencing reads. For example, in silico modelling can be used to predict sequences originating from proximal restriction sites (i.e., “predicted” sites). The predicted restriction fragment “map” for a given sample can be used as a comparison with actual data.

Therefore, the methods can include the step of in silico modelling to create a virtual restriction enzyme digest for a given nucleic acid sample. Restriction endonuclease digest mapping of a genomic sample in silico can be used as a control. For example, the in silico prediction of sequencing coverage for a given restriction endonuclease enzyme can be compared with actual sequencing coverage data. In some embodiments, differences between the in silico prediction of sequencing coverage and actual sequencing coverage can identify methylation, genic enrichment, GC bias, etc. Typically, only sequences reads with a mapping quality (MQ) score≧20, or a 99% chance of correct alignment, are included in these analyses.

In silico modelling for predicted RE enzyme cut sites can be used to produce a predicted digestion map for each nucleic acid sample. Predicted digestion maps can be used to identify the type and/or number of different enzymes that can be used. Preferably, the in silico modelling can identify the number and type of enzyme recognition sequences that will produce optimal sequencing coverage for a given nucleic acid sample.

b. DNA Fragment Size

The size of DNA fragments produced by digestion with blunt-cutting RE enzymes can influence coverage of DNA sequence reads and sequence mapping quality. Therefore, the number of non-overlapping restriction enzyme recognition sequences for each restriction enzyme within a given nucleic acid sample (i.e., restriction site density) can influence the coverage of DNA sequence reads and sequence mapping quality. The size of the RE enzyme recognition sequence can be directly proportional to the site density. Therefore, the relative sizes of RE enzyme recognition sequences can determine the relative site density and can influence the selection of RE enzymes, based on the desired experimental results. For example, a blunt-cutting RE enzyme that has a recognition sequence four base-pairs in length will produce a dense site profile across the genome but large amount of sequencing is required to obtain coverage on predicted sites. A six base pair cutting enzyme will produce a sparse site profile, but less sequencing will accomplish coverage saturation.

Typically, preferred sequence coverage can be achieved for digestion products of between 100 and 200 bp. The fragment size associated with preferred sequence coverage can vary in an enzyme-specific manner. Therefore, for some RE enzymes, coverage of predicted sites extends outwards to 400 bp or more. Generally, all enzymes show a reduction in the fraction of predicted sites with sequencing coverage for fragments greater than 400 bp. Therefore, in some embodiments, the RE enzymes are selected based on the DNA fragment size they will produce. Suitable methods for determining DNA fragment size are known in the art, for example, using a predicted digestion map by in silico modelling.

In some embodiments, the methods include digesting a nucleic acid sample by one or more blunt-cutting RE enzymes that cleave the nucleic acid sample to produce fragments of a specific length. Generally, the DNA fragments are less than 800 base pairs (bp) in length, preferably less than 400 bp, more preferably less than 200 bp, most preferably between approximately 100 bp and 200 bp in length. In some embodiments, RE enzymes can be selected that produce DNA fragments that are less than 100 bp in length, for example, 80 base pairs.

c. Genic Coverage

The methods include digesting a nucleic acid sample by one or more blunt-cutting RE enzymes that cleave the nucleic acid sample to produce fragments within genic regions of a genome. Predicted genic site fraction can vary from enzyme to enzyme for a given genomic sample. Therefore, in some embodiments, RE enzymes can be selected having predicted cut sites that produce DNA fragments with sequencing coverage in genic regions. Markers in genic regions are generally held to be more informative than non-genie markers as they are less repetitive and, for many studies, more likely to be in proximity of a trait-associated polymorphism.

3. Digestion of Nucleic Acid Samples

Digestion of nucleic acid samples (i.e., a first nucleic acid sample) by restriction endonuclease enzymes can be carried out by any means known in the art. Preferably, the digestion conditions are optimized to achieve maximum digestion of the nucleic acid sample. An exemplary digestion reaction typically includes bringing the nucleic acid sample into contact with one or more RE enzymes under manufacturer-specified conditions to allow for digestion. For example, enzymes can be added in a suitable buffer to form a reaction mix, at a suitable concentration, for a suitable time to allow for digestion of the nucleic acid by the RE enzyme(s). Preferably, restriction enzymes are added in an excess to ensure maximum digestion of nucleic acids. For example, enzymes can be added in a 2-fold, 3-fold, 4-fold, 5-fold, or more than 5-fold excess to the amount of DNA to achieve complete digestion. An exemplary incubation time is one hour, two hours, or more than two hours. An exemplary incubation temperature is 37° C. An exemplary incubation buffer contains 50 mM Potassium Acetate; 20 mM Tris-acetate (pH 7.9); and 10 mM Magnesium Acetate. The parameters can be optimized according to the specific requirements of different blunt-cutting RE enzymes. Typically, enzyme digestion is enhanced in the presence bovine serum albumin (BSA), or a similar protein, at a suitable concentration, for example, 100 μg/ml.

Digestion can include one type of blunt-cutting enzyme, or more than one type. For example, where it is desired that the same nucleic acid sample is cut at multiple different recognition sequences, a mixture of different blunt cutting RE enzymes can be used. In some embodiments, the nucleic acid sample is contacted with two, three, four, five, six, or more different RE enzymes.

B. Solid-Phase Immobilization

All of the described steps can be carried out using nucleic acid samples that are immobilized on a solid phase. Following addition to the sample, the solid-phase can be retained throughout the protocol until the post-adaptor ligation size selection step.

For example, nucleic acid samples can be hybridized to Solid-Phase Reversible Immobilization (SPRI) beads, and all method steps carried out using a bead-based methodology, for example, using samples micro-titer plates (Hawkins, et al., Nucleic acids research, 22(21):4543-4544 (1994)).

SPRI beads bind to the nucleic acid samples in a non-specific manner.

Immobilization can enhance the efficacy of digestion, reduce the loss of samples during washing and/or buffer exchange, and can facilitate the size-selection of digested nucleic acid samples. Genomic nucleic acid fragments can be immobilized on paramagnetic SPRI beads at any stage during the described methods, for example, before or after digestion of the nucleic acids with RE enzymes. DNA immobilized on the paramagnetic beads can be held in place during buffer exchange, DNA size selection and cleanup steps. Wash, elution, and hybridization buffers are known in the art, for example, as implemented by the Broad Institute (Fisher, et al., Genome biology, 12(1):R1 (2011)).

DNA samples can be hybridized to beads during all clean-up steps. Beads remain in solution during all stages of the protocol, but when the solution lacks PEG (poly ethylene glycol), the DNA can be in solution rather than hybridized to the bead.

The status of DNA hybridization in an exemplary protocol can be:
1) Initial, pre-enzymatic digestion cleanup. Hybridized.
2) Restriction enzyme digest. Not hybridized.
3) Post-restriction digest cleanup. Hybridized.
4) dA tailing. Not hybridized.
5) Post-dA tailing cleanup. Hybridized.
6) Adapter ligation. Not hybridized.
7) Post-adapter ligation size selection and clean-up. Hybridized during the second size selection step when fragments under 200 bp are removed.

1. Washing/Buffer Exchange

Nucleic acid fragments that are adhered or coupled to a substrate can be isolated from the solution and washed once or more as required to remove buffers and contaminants. For example, when SPRI magnetic beads are used, the beads and bound DNA can be separated from solution using a magnet. The isolated beads and bound DNA can be washed once, twice, or more than twice. Any suitable washing buffer can be used to remove non-bound contaminants from the beads.

In some embodiments, the use of solid-phase immobilization can replace column-based wash and manipulation steps. For example, samples bound to a solid phase can remain in the same vessel throughout the method steps. In other embodiments, washing of the DNA-SPRI beads is facilitated by use of a column. For example, the beads can be placed into a column and wash buffer passed through the column continuously to flush away the reaction mixture. The only remaining material bound to the beads is genomic DNA fragments.

In other embodiments, the wash steps are effectively integrated without employing a series of discrete cleanup-steps. For example, the SPRI beads can be added to the sample after the shearing step, and can remain in the reaction vessel throughout the sample preparation protocol. Thus, by allowing each cleanup step to employ the same beads, the described methods can greatly reduce the number of liquid-transfer steps required. The isolated, “purified” nucleic acids are then eluted from the surface of the beads. Preferably, this methodology increases the overall DNA yield, for example, by eliminating sample transfer steps and avoiding the loss of DNA sticking to the sides of the vessel or loss of volume in pipetting.

In some embodiments, DNA is selectively bound to the iron beads in the presence of a suitable binding buffer. An exemplary binding buffer is 20% polyethylene glycol (PEG), 2.5 M NaCl buffer. To wash the DNA/bead samples, the entire mixture vessel/tray can be placed on top of a magnet which pulls the beads and bound DNA to the sides of the well so that the reagents, washes and/or unwanted fragments can be removed with the supernatant.

Nucleic acid samples can be released from the capture surface of the solid phase (e.g., magnetic beads) using a suitable buffer. Preferably, DNA is eluted form the surface in a manner than minimizes loss and/or manipulation of the DNA. Exemplary methods and suitable buffers for eluting DNA bound to solid phase matrices are known in art. An exemplary incubation is in 10 mM Tris-HCl at 65° C. for 5 minutes, with agitation.

C. dA-Tailing

The methods preferably include the addition of an adenine moiety to the 3′ end of the DNA fragments dA tailing”). dA tailing prevents formation of self-assembled DNA concatemers following digestion of genomic DNA. In some embodiments, dA tailing enables compatibility of blunt-ended DNA fragments with “standard” DNA ligation adapters (e.g., Y adaptors with a dT overhang).

Methods for dA tailing are known in the art. For example, dA tailing can be achieved by contacting the nucleic acid fragments with the Klenow fragment of the DNA polymerase enzyme (3′-5′ exo-). Kits and materials for dA tailing are available from multiple commercial sources, including Applied Biological Materials, Inc. (Cat. No. E009); NEBNext® dA-Tailing Module; and New England Biolabs (Cat. No. M0212).

When DNA fragments are bound to a solid phase, such as SNIT beads, the DNA fragments can be eluted from the beads prior to the dA tailing step.

D. Ligation of DNA Sequencing Adapters

The methods can include ligation of the nucleic acid fragments to sequencing adapters, to produce adapter-ligated DNA fragments.

Restriction enzymes used with this protocol produce blunt-end, 5′ phosphorylated DNA fragments, amenable to ligation with standard universal sequencing adapters. The blunt-ended DNA fragments including an adenine moiety at the 3′ end of each strand are modified to facilitate sequencing or microarray analysis, for example, by ligation to appropriate sequencing adapters.

Typically, the adapters are universal adapters (i.e., the adapters are not specific to the overhang region produced by any specific RE enzyme) (Shin, et al., Nature Neuroscience 17, 1463-1475 (2014)).

In some embodiments, the adapters include a dT overhang complementary to the dA tail added to the blunt-ended nucleic acid fragment as described above. Therefore, methods for ligating sequencing adapters to genomic DNA fragments that do not require PCR are provided. For example, blunt-ended DNA fragments can be directly ligated to adaptors for high throughput sequencing. Ligation to adapters does not require end-repair of nucleic acid fragments, when the fragments are created using a blunt-end RE enzyme.

The methods enable the use of universal adapters, such as ILLUMINA® Y-adaptors, for subsequent next generation sequencing applications. For example, the methods enable paired-end sequencing of size-selected nucleic acid fragments. Exemplary adaptors that can be used are well known in the art and include, for example, ILLUMINA® adaptors. In some embodiments, the universal adapters are standard ILLUMINA® Y adapters.

Methods for ligation of nucleic acid fragments to universal sequencing adapters are known in the art. For example, the methods can include ligation of adapters to DNA fragments mediated by a DNA ligase enzyme that can mediate the ligation of blunt DNA ends. Exemplary polydeoxyribonucleotide synthase (ATP) enzymes include T3 DNA ligase and T4 DNA ligase.

Kits and materials for the ligation of blunt DNA ends are available from multiple commercial sources, such as New England Biosciences (Cat. No. M0317L; M0202M).

E. Complexity Reduction

In some embodiments, the methods include the step of enriching fragments of the desired sequence(s) from the pool of digested DNA fragments. The step of sample enrichment is optional, and reduces the complexity of a sample, such as genomic DNA, to generate of a subset of the sample. This subset can be representative for the whole (i.e., genomic DNA) sample and is preferably a reproducible subset. When one or more of the same or similar samples are reduced in complexity using the same methods, the same, or at least comparable, subsets are obtained.

The methods can include one or more steps for reducing the complexity of the nucleotide sequences in the first nucleic acid sample. In some embodiments, methods for reducing the complexity of nucleotide sequences in a nucleic acid sample include capturing target polynucleotides from an initial nucleic acid sample to obtain a nucleic acid sub-population having desired sequences, or lacking certain undesired sequences. In some embodiments, an initial nucleic acid sample includes target polynucleotides and non-target polynucleotides.

The methods used for complexity reduction may be any method for complexity reduction known in the art. Representative methods include size selection and sequence selection. Size and/or sequence selection can be carried out using methods known in the art, for example, using affinity-mediated isolation of specific sequences or sizes of nucleic acids, or electrophoresis methods, etc. Examples of methods for complexity reduction of nucleic acid samples are described in EP 0 534 858; US 20140243232 A1; WO 03/012118; and WO 00/24939.

An exemplary method for enrichment of the desired DNA sequences is size-selection (i.e., molecular weight exclusion) by, for example, selective hybridization to a solid-phase matrix, such as beads. The beads can be magnetic beads, such as SPRI beads. SPRI beads are an alternative to gel extraction for size selection and purification of DNA fragments from a pool or library of DNA fragments before amplification. If added to the DNA in the presence of polyethylene glycol (PEG) and salt (e.g., NaCl), SPRI beads replace the capricious gel extraction with standardized and quick binding and elution steps. Thus, in some embodiments the described methods include SPRI-based in-solution size selection of DNA fragments. Methods for selective hybridization to SPRI beads are known in the art. For example, molecular weight exclusion, which is essentially a size selection, of unwanted lower molecular weight DNA fragments can be controlled through the volume of the PEG NaCl buffer that is added to the reaction, changing the final concentration of PEG in the resulting mixture and altering the size range of fragments bound to the beads. DNA fragments that have been cleaned or size selected are eluted from the beads, ready for the next step; however, the eluate does not have to be transferred into a new reaction vessel. Rather, the reagents for the next step can be added directly to the reaction vessel containing samples and beads. Typically, the presence of beads does not interfere with any of the steps in the process. This with-bead protocol has greatly increased the number of unique fragments entering the pond PCR step, increasing the complexity of libraries made by roughly 12-fold.

The ability to control the smallest size of the nucleic acid fragments that are bound to the beads enables the selective isolation of libraries of digested DNA fragments from a solution contaminated with non-ligated adaptors and adaptor-dimers. In general, the greater the concentration of PEG and salt in the solution, the smaller the size of nucleic acid fragments that are bound, and therefore the lower the starting molecular weight of the isolated products.

The ability of DNA fragments of a given size to hybridize to beads can be dependent upon the composition of the hybridization buffer used. For example, DNA fragments below a certain size will fail to hybridize to the beads according to the concentration of polyethylene glycol (PEG) in the hybridization buffer. Since the digested DNA fragments will always be longer than the size of the two adaptors at the ends of the DNA insert, fine-tuning the concentrations of these compounds to exclude the characteristic size of adaptor-dimers from binding to the beads will in effect purify the library.

Therefore, the methods can include the step of enriching the digested nucleic acid sample for DNA fragments within a certain size range by hybridization to beads. In some embodiments the size range of nucleic acid fragments that are coupled to the hybridization beads is varied by manipulation of the composition of the hybridization buffer. The selective hybridization of DNA fragments on the basis of size can be carried out using any apparatus known in the art. Suitable methods and apparatus can be determined according to the desired results. For example, Solid-Phase Reversible Immobilization (SPRI) methodology enables column-free cleanup of samples and gel-free size selection of DNA fragments, which makes it highly amenable to robust, large-scale multiplexing. In other embodiments, a conventional column/gel method is optimal.

Sizing of DNA fragments by SPRI concentration can give rise to a spectrum of DNA fragment sizes. Typically, DNA fragments fractionated by the described methods are greater than 100 base pairs, for example, 200 base-pairs or more. Exemplary size ranges for DNA fragments enriched by the described methods are between approximately 100 nucleotides and 1,000 nucleotides. In some embodiments the fragments are between 200 and 600 nucleotides. For example, ligation products with a lower size limit of 200 bp (i.e., approximately 80 bp of genomic DNA fragment, plus adaptors) and an upper limit of 800 bp, (i.e., approximately 680 bp of genomic DNA fragment, plus adaptors). Therefore, in some embodiments the methods for the DNA fragments below the expected size and a variable upper size limit for DNA fragments that tended to be below 680 bp. Typically, the enriched DNA fragments contain little or no adaptor dimer contamination. Samples can be eluted from the beads using any suitable elution buffer, for example, samples can be eluted in 10 mM Tris-HCl. The eluted double-stranded DNA fragments preferably include between 200 and 800 nucleic acids.

F. Addition of Sequence Identifiers

The methods include a step to amplify the sequenceable portion of the library and to add labels for sample identification of DNA fragments following multiplexing. The addition of a label to the adapter-ligated nucleic acid sample enables it to be distinguished from a second or more nucleic acid sample. The adapter-ligated nucleic acid sample can be an enriched nucleic acid sample (i.e., if the methods include complexity reduction) or a non-enriched nucleic acid sample. The inclusion of sequence identifiers (e.g., a “barcode” of between 4 and 10 nucleotides) to DNA fragments enables rapid and reliable identification of samples of different origins, e.g. obtained from different plant lines, when the enriched samples of from two or more different nucleic acid samples are combined. For example, by incorporating different sequence identifiers (i.e., “bar codes”) for different starting material or different assays, large numbers of fragments from different starting material and/or assays can be amplified and/or sequenced as pool and linked to a specific profile by identifying the bar code during bioinformatics analysis. Therefore, different sequence identifiers are used for each original nucleic acid sample. For example, a first set of sequence identifiers can be added to a first adapter-ligated, enriched nucleic acid sample, and a second (different) set of sequence identifiers (bar-codes) can be added to a second adapter-ligated, enriched nucleic acid sample. Therefore, when two, three or more nucleic acid samples are used, two, three or more differently bar coded adapter-ligated enriched nucleic acid samples are produced, corresponding to each of the respective first, second, third or more original samples.

Typically, the amplification step includes a polymerase chain reaction (PCR) reaction. For example, when standard ILLUMINA® Y adapters are used, the primers including the sequence identifiers can be added to the adapters. Therefore, methods for adding sequence identifiers to the adapter-ligated enriched DNA sample include contacting the adapter-ligated enriched DNA sample with one or more oligonucleotide primers under hybridizing conditions.

The PCR can be optimized to reduce GC bias and prevent incorporation of errors into the DNA. For example, the PCR can be carried out using a low number cycles. Therefore, in some embodiments, the PCR is carried out using less than 10 cycles, for example 5-6 cycles. Techniques and reagents for PCR are known in the art. Exemplary PCR reagents for minimal PCR bias in AT and GC-rich regions of DNA templates include the Kapa HIFI PCR reagents (e.g., Kapa Biosystems, cat. no. KK2502).

In some embodiments, barcoding is performed using a dual-indexing system. An exemplary protocol for dual-indexing is the TruSeq Dual Index Sequencing Primer Box (Lamble. et al., BMC biotechnology, 13:104 (2013)). While the TruSeq Dual-Index Sequencing Primer Box (FC-121-1003, Illumina) offers compatibility with up to 96 libraries, much higher levels of multiplexing are possible with custom primers.

Oligonucleotide primers with custom indices can be selected with input from the user's sequencing center to ensure compatibility with local protocols. In some embodiments, samples are barcoded and are then bound to the surface of a solid phase matrix, such as SPRI beads, and samples washed to remove primers and other non-desired nucleic acid fragments or contaminants.

G. Sequencing

The described methods can include sequencing and associated data analysis. Suitable method of sequencing of the complexity-reduced, enriched nucleic acid sample are known in the art. Exemplary sequencing methods include the dideoxy chain termination method. In some embodiments, nucleic acid sequencing is performed using high-throughput sequencing methods, such as described in WO/2003/004690, WO/2003/054142; WO/2004/069849; WO/2004/070005; WO/2004/070007, and WO/2005/003375.

Sequences can be aligned to provide an alignment. Methods to align sequences for comparison purposes are well known in the art. For example, sequences can be aligned to a corresponding genomic map, such as a blunt-cutting RE enzyme digestion map for the corresponding blunt-cutting RE enzyme(s) used in the digestion step, for example, produced by in silico modelling. The NCBI Basic Local Alignment Search Tool (BLAST) (Altschul, et al., 1990) is available from several sources, including the National Center for Biological Information (NCBI, Bethesda, Md.) and on the Internet, for use in connection with the sequence analysis programs blastp, blastn, blastx, tblastn and tblastx. Generally, alignment of nucleic acid sequences is carried out using sequenced data corresponding to enriched, adapter ligated nucleic acid fragments in the absence of sequences associated with the adaptors, oligo-nucleotide primers and/or sequence identifiers. Therefore, sequence reads represent at least a portion of the corresponding nucleic acid sample. The sequenced data can be used to identifying the origin of the enriched nucleic acid fragment.

Typically, dA-tailing of the blunt-ended DNA fragments produces minimal chimeric sequencing reads due to concatamer formation. Thus, the methods are suited to paired-end sequencing. Preferably the paired-end reads align correctly to a genome to a greater extent than single end reads. Paired reads are generally held to be more likely to map correctly than unpaired reads (Li, et al., Genome research, 18(11):1851-1858 (2008)).

Typically, at least a portion of enriched, adapter-ligated nucleic acid sample is sequenced. In preferred embodiments, the amount of overlap of sequenced fragments from different nucleic acid samples is at least 50%, at least 60%, at least 70%, or at least 80%.

Evaluation of the effect of paired versus single end reads on alignment can be assessed by any suitable means known in the art. For example, effect of paired versus single end reads on alignment can be assessed by a comparison of the mapping quality of reads. Mapping quality (MQ) is a measure of confidence in a given read alignment, given the information available in the reference genome. MQ is a Phred scaled value. For example, an MQ of 20 indicates a 1 in 100 chance of misalignment, and a MQ of 30 indicates a 1 in 1000 chance. Reads that map equally well at multiple locations or fail to map at all are given mapping qualities of 0. For many experiments, alignments below a certain mapping quality, usually values of 20, 30 or 40, are filtered out.

Sequences from each enzyme dataset can be aligned as both paired and unpaired reads to the maize and rice reference genomes and the fraction of reads aligning at mapping quality MQ≧20 and MQ≧30 determined.

In some embodiments, the sequencing is carried out using nucleic acid samples bound to a solid phase, such as a bead. Sequencing can include the step of annealing the adapter-ligated enriched DNA fragments to the beads. The beads can be located within the wells of a multi-well plate, for example, a 96-well plate. Each well of the plate can contain a single bead, or a multiplicity of beads bound to the adapter-ligated enriched DNA fragments.

H. Imputation

One important challenge of low-coverage next generation sequencing methodologies is that of missing data, both missing markers and missing alleles due to low coverage. A sequencing read represents a discrete measurement of a single allele, and coverage at a given marker is expected to follow a binomial distribution. When only one allele is recovered at a heterozygous site, the result is a falsely homozygous call, which is likely to occur when the coverage is low. Algorithms can be employed to resolve these issues in biallelic populations through the use of variable emission probabilities based on sequencing coverage at a given site. By adjusting the probability of a state based on the number of sequencing reads assigned to each allele, missing and erroneous calls can be better imputed and corrected than existing methods with high accuracy, even at very low levels of coverage.

In the case of datasets from organisms with non-existent or incomplete reference genomes, namely ones that exist as unscaffolded contigs, algorithms designed for humans fail entirely. Imputation methods do exist that are suitable for these datasets that can provide high levels of accuracy (Rutkoski, et al. G3 (Bethesda), 3(3):427-439 (2013); Stekhoven, et al., Bioinformatics, 28(1):112-118 (2012)).

While differing in implementation, these methods consistently rely on identifying proximal markers through linkage disequilibrium. As such, an initial dataset with only a modest number of missing markers is advisable when employing these methods. In addition, data with a high error rate may be unsuitable for these algorithms.

Imputation methods designed for GBS are implemented to incorporate parental data into phasing and, when necessary, impute missing parental genotypes from population data. Further, they do not assume Hardy-Weinberg equilibrium or random mating, as may be the case with many populations. Many, however, are designed to work with NAMs or other populations without heterozygosity (Poland, et al., PloS one, 7(2):e32253 (2012); Spindel, et al., Theoretische and angewandte Genetik, 126(11):2699-2716 (2013); Huang, et al, Genetics (2014); Andolfatto, et al., Genome research, 21(4):610-617 (2011)).

Of the GBS capable imputation methods that do exist, most are designed for inbred lines where heterozygosity is largely absent. For populations with large remaining amounts of heterozygosity, these methods are unsuitable. While a reduced representation sequencing (RRS) experiment is restricted to a subset of the total alleles in a population by design, it is unlikely that the entire subset will be recovered in each sample.

The described methods can include algorithms for imputation of missing data, particularly when low-coverage sequencing resulting in missing data.

In some embodiments, missing data occurs when sequencing coverage is insufficient to type every allele in each sample. For example, the proportion of unrecovered alleles increases with the level of multiplexing. The missing data can manifests in multiple forms. In some embodiments, no alleles are recovered at a marker in a given sample, resulting in the absence of any genotype at that site. In other embodiments, not all alleles are recovered at a sample's marker. If alleles at this site are monomorphic, no data is lost. If the marker is heterozygous in the sample, however, that site will be falsely identified as a homozygote (Swarts, K., et al., The Plant Genome, (2014)).

It is these erroneous homozygote calls that pose the greatest challenge for the imputation of missing data in low coverage sequencing datasets.

Methods of data imputation for RRS methods are known in the art. For example, in some embodiments, missing variant states are not directly imputed. Regions can be classified as heterozygous, or homozygous for a given genotype. Variants can first be phased by parental states, then a most likely state (e.g., homozygous, or heterozygous) is determined. For example, a sliding window of a fixed number of bases (e.g., 5 mega base pairs) across the genome using a least squares based method can be applied. In some embodiments the methods can be described using the equation:

$$S = {\sum }_{i = 1}^{n} {r}_{i}^{2}$$

Where S is the sum of residuals, and r is the residual defined by the equation:

rᵢ=gᵢ−mᵢ

Where gᵢ is the window genotype and mᵢ is the individual marker's genotype.

All possible marker genotypes, are assigned values of 0, 1, and 2 respectively. Each possible “overall” genotype is assigned a value using the same system, and each of the three possible genotypes is tested against the set of markers. The genotype with the lowest sum of squared residuals is assigned to the window.

In some embodiments, where windows less than ten total variants exist, variant states in proximal windows can be included. Recombination breakpoints are resolved by first identifying proximal bins with differing calls. A five marker sliding window can be then moved across the two proximal bins in a forward and reverse direction and a genotype call obtained at each point. When the window is transitioned from the first bin's genotype to the second's and vice versa, the point is recorded. Finally, the mean value of the two transition points is used as the point of recombination.

These methods can be employed to resolve heterozygous regions in GBS data in spite of the high rate of missing and erroneous data, especially false homozygous calls resulting from low coverage of heterozygous SNPs.

### III. Uses

The disclosed methods can be employed in a broad range of applications and analyses. For example, in some embodiments, the methods are employed in population genetic analyses.

The methods can be used with next generation sequencing techniques to generate large datasets, for example, for population genomics. The described modified GBS methods selectively isolate the same part of the genome of multiple individuals, and provide methods for identification of the sequence associated with each individual. Therefore, the methods can be used to provide a comparison of the same DNA fragment or multiplicity of fragments from a population of individuals. Because the methods are compatible with numerous blunt-end RE enzymes, the parameters of the sequence selection can be modified according to the needs of a given experiment.

In some embodiments the steps of digesting a first nucleic acid sample with one or more blunt-cutting restriction endonuclease (BRE) enzymes; incorporating a non-templated dAMP on the 3′ end of the blunt DNA fragment; ligating the DNA fragments to universal adapters; optional complexity reduction by selecting DNA fragments on the basis of size; and labelling the optionally size-selected fragments with sequence identifiers is carried out consecutively or simultaneously with a second or more nucleic acid sample. For example, the methods can be carried out consecutively or simultaneously on a second sample, or a third, fourth, fifth, sixth, etc. Therefore, in some embodiments, the methods are consecutively or simultaneously carried out on 10, 100, 1,000 or more than 1,000 samples. Typically, the samples are genomic DNA samples, such as total genomic DNA.

Generally, the methods are most useful when the methods are consecutively or simultaneously carried out using the same methods under the same, preferably identical, conditions for a multiplicity of nucleic acid samples. For example, a first nucleic acid sample can be processed in the same manner as a second or more nucleic acid sample. In some embodiments the methods are carried out at using a multiplicity of different samples at the same time, for example, using a 96-well plate or similar platform.

Therefore, equivalent or comparable components of the nucleic acid samples will be retained in the multiplicity of corresponding enriched nucleic acid samples.

Typically, the multiplicity of nucleic acid includes samples that are related to one another. For example, a first nucleic acid sample can be related to a second or more nucleic acid sample. For example, the first nucleic acid sample and the second or more nucleic acid sample can belong to different species of the same or different genus. In one embodiment, the first nucleic acid sample and the second or more nucleic acid samples belong to different varieties or sub-species of the same species, or to different individuals from the same variety or sub-species. Exemplary nucleic acid samples include whole genomic DNA belonging to a plant, animal, bacteria, virus or fungi. In some embodiments the first nucleic acid sample and the second or more nucleic acid sample can belong to different humans.

Many popular imputation algorithms are designed specifically for human data (Li et al., Annual review of genomics and human genetics, 10:387-406(2009)). These methods often assume high per-marker accuracy, complex haplotype, and the availability of a reference genome. GBS datasets, on the other hand, may have significant amounts of missing or inaccurate data. Haplotypes may be complex in some cases, but in many experiments parental data will be available and genotypes can be phased in a straightforward manner. Reference genomes are often not available or are incomplete. While popular methods such as fastPHASE can be applied to GBS data (Stephens, et al., American journal of human genetics, 73(5):1162-1169 (2003); Scheet, et al., American journal of human genetics, 78(4):629-644 (2006)); Marchini, et al. Nature reviews Genetics, 11(7):499-511 (2010)), pre-processing is advisable. Pre-processing should test for false homozygotes resulting from low coverage and collapse non-independent markers into single values. Non-independent markers are polymorphisms called from a set of reads aligned to the same location, which is typical with GBS experiments. Errors, including misalignment, false homozygosity, and paralogous sequence will be common to all markers originating from this set of reads. Improperly accounted for, they may offer multiple, seemingly independent confirmations of a false genotype that may produce an incorrect result from imputation. Thus, it is recommended that all markers from the same set of reads be treated as a single event rather than independently.

GBS has already demonstrated viability in trait mapping, admixture analysis, genome wide association, population genomics, and characterization of diversity in reference and non-reference organisms (Narum, et al., Molecular ecology, 22(11):2841-2847 (2013)). The modifications described here increase the portability of GBS to individual labs interested in adopting it by reducing the initial cost of oligos, allowing for simple, low-cost, pilot experiments, and integrating library preparation more directly into the standard ILLUMINA® pipeline.

### IV. Compositions

The compositions described below include materials, compounds, and components that can be used for the disclosed methods. Various exemplary combinations, subsets, interactions, groups, etc. of these materials are described in more detail above. However, it will be appreciated that each of the other various individual and collective combinations and permutations of these compounds that are not described in detail are nonetheless specifically contemplated and disclosed herein. For example, if one or more restriction endonuclease enzymes that cleave DNA to yield blunt-ends are described and discussed and a number of substitutions of one or more of the enzymes, each and every combination and permutation of the enzymes possible are specifically contemplated unless specifically indicated to the contrary. These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Nucleic Acid Samples

For the described methods, samples generally can be collected and/or obtained in any of the manners and modes in which nucleic samples are collected and obtained.

By “sample” is intended any sampling of nucleic acids. Any nucleic acid sample can be used with the described methods. Examples of suitable nucleic acid samples include genomic samples, RNA samples, cDNA samples, nucleic acid libraries (including cDNA and genomic libraries), whole cell samples, environmental samples, culture samples, tissue samples, bodily fluids, and biopsy samples. Numerous other sources of nucleic acid samples are known or can be developed and any can be used with the described method. Preferred nucleic acid samples for use with the described method are nucleic acid samples of significant complexity such as genomic DNA samples, preferably whole genomic DNA and dsDNA libraries created by enzymatic or mechanical cleavage of genomic DNA. Therefore, nucleic acid samples can be one or more genomic DNAs or a pool of multiple genomic DNAs, or a DNA sequencing library.

In certain embodiments, the nucleic acid sample is genomic DNA, such as human genomic DNA. Human genomic DNA is available from multiple commercial sources (e.g., Coriell #NA23248). Nucleic acid fragments are segments of larger nucleic molecules. Nucleic acid fragments, as used in the described method, generally refer to nucleic acid molecules that have been cleaved. A nucleic acid sample that has been incubated with a nucleic acid cleaving reagent is referred to as a digested sample. A nucleic acid sample that has been digested using a restriction enzyme is referred to as a digested sample. Therefore, nucleic acid samples can be genomic DNA, such as human genomic DNA, or any digested or cleaved sample thereof. In a preferred embodiment, the nucleic acid sample contains one or more genomic DNA fragments of interest.

In some embodiments the nucleic acid sample includes more than a single genomic DNA sample. For example, a nucleic acid sample can include genomic DNA from two different organisms, or more than 2 organisms, such as 10, 100, 1,000 or more than 1,000 different organisms. When nucleic acid samples contain genomic DNA from more than a single organism, the organisms can be from the same species, or different species. In some embodiments, genomic DNA samples include the genomic DNA representative of an entire population of organisms. The genomic DNA within each sample may represent the complete genome for an organism, or may be incomplete, for example, a representative fraction of a library of genomic DNA fragments.

Generally, an amount of genomic DNA between 1 ng and 1 μg is required per sample, for example, 500 ng of genomic DNA per sample.

In some embodiments, nucleic acid samples are bound to a solid phase. For example, nucleic acid samples can be hybridized onto beads, such as AMPure XL SPRI beads.

In some embodiments, the nucleic sample includes nucleic acids isolated from more than one individual. For example, the nucleic acid sample can include nucleic acid (e.g., DNA), for across a population or other collection of individuals. In preferred embodiments, the population are somehow related. Relatedness means that genotypes found in one individual may be found in others, and makes validation of true genotypes easier (since false ones are likely to only be found in one individual). In some embodiments, the nucleic acid samples are population wide genomic samples. For example, the nucleic acid sample can be a trait mapping population that includes DNA samples from all members of an F₂ cross. Tested examples include the F₂ offspring of an F₁ selfing of a B73 xCountry Gentleman cross. DNA was extracted from all samples and sequenced via GBS. The DNA can be a sampling of wild populations, for example, a collection of DNA samples from mosquitos found in the same region.

B. Blunt-Cutting Restriction Endonuclease Enzymes

Restriction endonucleases (RE) are enzymes that cut the sugar-phosphate backbones of complementary nucleic acids within the DNA double helix to produce blunt-ended nucleic acid fragments (i.e., both strands terminate in a base pair). Restriction endonuclease enzymes that recognize a specific sequence of nucleotides and cut both strands of DNA to yield blunt-ended DNA fragments are well known in the art.

A genomic DNA sample digested by a blunt-cutting RE enzyme will be digested by a particular restriction endonuclease into a distinct profile of blunt-ended DNA fragments (i.e., restriction fragments). Recognition sequences for restriction endonuclease enzymes are generally between 4 and 8 bases. Therefore, the amount of bases in the recognition sequence can determine how often the recognition site for a given enzyme appears within any given genome. The number of non-overlapping recognition sites within any given nucleic acid sequence can be directly proportional to the number of DNA fragments produced by the enzyme.

Restriction endonuclease enzymes that digest double stranded DNA to produce a blunt-ended DNA fragments (i.e., blunt-cutting RE) can recognize palindromic or non-palindromic sequences. The cut site can be within the recognition sequence, or can be contiguous with the recognition sequence, or at a distance from the recognition sequence. The blunt ends produced by blunt-cutting RE are compatible with universal sequence adapters.

A non-limiting list of blunt-end restriction endonuclease enzymes includes AanI, Acc16I, AccBSI, AccII, AcvI, AfaI, AfeI, AhaIII, AjiI, AleI, AluBI, AluI, Aor51HI, Asp700I, AssI, BalI, BbrPI, BmcAI, BmgBI, BmiI, BoxI, BsaAI, BsaBI, Bse8I, BseJI, Bsh1236I, BshFI, BsnI, Bsp68I, BspFNI, BspLI, BsrBI, BssNAI, Bst1107I, BstBAI, BstC8I, BstFNI, BstPAI, BstSNI, BstUI, BstZ17I, BsuRI, BtrI, BtuMI, Cac8I, CdiI, CviJI, CviKI_1, CviRI, DinI, DpnI, DraI, Ecl136II, Eco105I, Eco147I, Eco32I, Eco47III, Eco53kI, Eco72I, EcoICRI, EcoRV, EgeI, EheI, EsaBC3I, FaiI, FnuDII, FspAI, FspI, GlaI, HaeI, HaeIII, HincII, HindII, HpaI, Hpy166II, Hpy8I, HpyCH4V, KspAI, LpnI, MalI, MbiI, MlsI, MluNI, MlyI, MroXI, MscI, MslI, Msp20I, MspA1I, MssI, MstI, MvnI, NaeI, NIaIV, NruI, NsbI, NspBII, OliI, PceI, PdiI, PdmI, PmaCl, PmeI, PmlI, Ppu2II, PshAI, PsiI, PspCI, PspN4I, PvuII, RruI, RsaI, RseI, ScaI, SchI, SciI, SfoI, SmaI, SmiI, SmiMI, SnaBI, SrfI, SseBI, SspD5I, SspI, Sth302II, StuI, SwaI, XmnI, ZraI, and ZrmI.

These enzymes can be isolated from bacteria and archaea. Blunt-cutting RE enzymes are available from multiple commercial sources (for example, from New England Biolabs, cat nos. R0610; R0137; R0167; R0195; R0187; R0108; and R0103).

Preferably, restriction endonucleases produce DNA fragments with the appropriate sequence at the expected cut-site with a high degree of reproducibility. For example, restriction endonucleases can produce DNA fragments with the appropriate cut sites in 80% or more, preferably 90%, or more preferably greater than 90% of the DNA fragments within the genomic DNA samples.

Generally, blunt-cutting RE enzymes can be present in reaction mixtures at a concentration ranging from approximately 1 unit to 20 units of enzyme per μg of DNA. An exemplary concentration for each enzyme is between 10 and 20 units for genomic DNA in a 1 hour digest. Generally, enzymes are stored in a glycerol solution to prevent enzyme activity. Therefore, the volume of the enzyme stock solution added to the digestion reaction should not exceed 10% of the total reaction volume to prevent star activity due to excess glycerol.

C. Solid-Phase Reversible Immobilization (SPRI) Beads

Solid-Phase Reversible Immobilization (SPRI) beads are magnetic particles coated with carboxyl groups (in the form of succinic acid) that can bind DNA non-specifically and reversibly. Exemplary beads measure approximately 1 μM in diameter, are super-paramagnetic and display iron as well as the carboxyl groups on the surface. The iron and negative charge surface features mediate the interaction between nucleic acid and the solid-phase surface.

SPRI beads are available from multiple commercial sources, for example, AMPure XL SPRI beads (Beckman Coulter cat. No. AG3880).

Preferably, SPRI-bead-based nucleic acid interactions are implemented in a standard microtiter plate, such as a 96-well plate format (Fisher, et al., Genome Biology 2011, 12:R1 doi:10.1186/gb-2011-12-1-r1). The 96-well plate format replaces single tube spin-column-based cleanups with liquid handling-compatible magnetic bead-based cleanups; enables selection of molecular weight ranges, eliminating the need for agarose gel-based sizing; and simplifies the process by allowing elimination or combining of several steps, which results in a higher overall DNA yield.

Suitable buffers for enhancing binding of SPRI beads to nucleic acids are known in the art. An exemplary binding buffer is 20% PEG 8,000, 2.5 M NaCl. Buffers can be manipulated for hybrid selective capture can be used. Suitable buffers, reagents and vessels, such as microtiter plates, are available from multiple commercial sources.

D. Adapters

Adapters are described for use with the methods. Typically, adaptors are double-stranded DNA molecules or about 10 to about 40 nucleotides, designed to be ligated to the ends of DNA fragments. Adaptors can include two partially complementary oligonucleotides that align to form a double stranded molecule that is compatible with the end of a DNA fragment generated by a blunt RE enzyme.

Preferably, the adapters are universal adapters that can be used to bind to DNA fragments produced by any blunt-cutting restriction endonuclease enzyme. Universal adapters are compatible with the blunt ended DNA fragments created by all blunt-cutting RE enzymes. In some embodiments, the adapters are compatible with any double stranded DNA fragment having a single base overhang. For example, universal adapters can have a single-base overhang that is complementary to a single base overhang that is common to a pool of double stranded DNA fragments. In some embodiments, the universal adapters are compatible with all DNA fragments having a single adenine

Preferred universal sequencing adapters are “Y-shaped” adapters (Y-adaptors). Y adapters allow different sequences to be annealed to the 5′ and 3′ ends of each molecule in a library (Shin, et al., Nature Neuroscience 17, 1463-1475 (2014)). The arms of the Y are unique, and the middle part, connected to the DNA fragment, is complementary. Therefore, Y-adaptors reduce concatamer formation due to the dA tailing step, which in turn improves the quality of paired-end sequencing data. Ligation of dA-tailed inserts with Y-adapters enables synthesis of two unique adapters on either end of a blunt ended DNA sequence. Thus, Y-shaped adapters also allow for paired-end sequencing. Fragments have a unique sequence on either end, which allows for the first “run” to sequence one side of all molecules, then synthesize the reverse and sequence that.

In some embodiments, the sequencing adapters are ILLUMINA® Y-adaptors, paired with the dA tailing step, prevent concatamer formation, increase the sequenceable fraction of the library, and allows for paired-end sequencing. Use of ILLUMINA® Y-adaptors also enables incorporation of dual-indexed barcodes during library amplification, which facilitates large-scale, inexpensive multiplexing. In some embodiments, the adapters enable selective PCR enrichment of adapter-ligated DNA fragments. Preferably, sequence adapters can bind to a flow cell. Therefore, the sequence adapters enable the associated DNA fragments to be manipulated through multiple applications for next generation sequencing.

The ligation of sequencing adapters to nucleic acid fragments can require an enzymatic reaction catalyzed by a ligase enzyme in which two double-stranded DNA molecules are covalently joined together. Ligation enzymes are known in the art.

In some embodiments, all or part or the adaptor sequence can be used as a template sequence for one or more PCR oligonucleotide primers. For example, adapters and/or oligonucleotide primers can be designed to be complementary. Exemplary nucleic acid sequences for each complementary strand of a universal adapter are

(SEQ ID NO. 1)
5′-/5Phos/GATCGGAAGAGCACACGTCTGAACTCCAGTCAC-3′;
and
(SEQ ID NO. 2)
5′-ACACTCTTTCCCTACACGACGCTCTTCCGATCT-3′.

E. Sequence Identifiers

In some embodiments, the nucleic acid fragments include sequence identifiers (i.e., indexing or “barcoding” regions). Sequence identifiers can identify the origin of any given nucleic acid fragment upon further processing. In the case of combining processed products originating from different nucleic acid samples, the different nucleic acid samples should be identified using different tags. Exemplary sequence identifiers include a nucleotide sequence of varying but defined length that is uniquely used for identification of one or more specific nucleic acid samples.

Sequence identifiers can be added to a nucleic acid sample by any means known in the art. For example the addition of sequence identifiers to oligonucleotide primers (i.e., “Barcoding”) enables unique labelling of DNA fragments within a pool of multiple samples. Barcoding enables multiple DNA libraries to be pooled together into a single sequencing lane (i.e., multiplexing).

In certain embodiments, each DNA sequence adapter contains more than a single unique sequence which enables identification of each adapter-ligated DNA fragment as belonging to certain organism, library, pool of libraries, etc., through a dual-index system. In some embodiments each unique barcode sequence is between 4 and 10 nucleotides in length, inclusive. The length of the sequence identifier can be adjusted according to the needs of the user. For example, a length of 4 nucleotides is sufficient to produce up to 256 different sequences. Preferably, the tag sequence identifiers differ by at least one nucleotide amongst all the different samples. An exemplary sequence identifier is 6 nucleotides in length.

Typically, sequence identifiers (i.e., barcodes) are included within oligonucleotide primers. Therefore, each double-stranded DNA fragment includes at least two unique barcodes, one at each end of the adapter-ligated DNA fragment. The adapters can be designed to be ligated to DNA fragments by hybridization to a known sequence by a standard polymerase chain reaction (PCR), such as a low-cycle PCR. Therefore, oligonucleotide primers including (1) at least a portion of the same nucleotide sequence as the terminal parts of universal adapter and (2) a distinct sequence identifier are provided. The oligonucleotide primers can be designed for use with adapters bound to a specific subset of fragments, or to all adapters, as required by the needs of the experiment. Methods for designing and producing appropriate pairs of oligonucleotide primers including one or more sequence identifiers for use in PCR for amplifying adapter-ligated nucleic acid fragments are known in the art. Custom-designed oligonucleotide primers are available from multiple commercial sources.

In some embodiments, universal adapters and/or oligonucleotide primers can be designed to be complementary. Exemplary oligonucleotide sequences for a complementary oligonucleotide primers that can hybridize to the universal adapter of SEQ ID NO. 1 and SEQ ID NO. 2 are

(SEQ ID NO. 3; reverse primer)
5′-CAAGCAGAAGACGGCATACGAGAT[NNNN]GTGACTGGAGT
TCAGACGTGTGCTCTTCCGATC*T-3′;
(SEQ ID NO. 4; reverse primer)
5′-CAAGCAGAAGACGGCATACGAGAT[NNNNN]GTGACTGGAG
TTCAGACGTGTGCTCTTCCGATC*T-3′;
(SEQ ID NO. 5; reverse primer)
5′-CAAGCAGAAGACGGCATACGAGAT[NNNNNN]GTGACTGGA
GTTCAGACGTGTGCTCTTCCGATC*T-3′;
(SEQ ID NO. 6; reverse primer)
5′-CAAGCAGAAGACGGCATACGAGAT[NNNNNNN]GTGACTGG
AGTTCAGACGTGTGCTCTTCCGATC*T-3′;
(SEQ ID NO. 7; reverse primer)
5′-CAAGCAGAAGACGGCATACGAGAT[NNNNNNNN]GTGACTG
GAGTTCAGACGTGTGCTCTTCCGATC*T-3′;
(SEQ ID NO. 8; reverse primer)
5′-CAAGCAGAAGACGGCATACGAGAT[NNNNNNNNN]GTGACT
GGAGTTCAGACGTGTGCTCTTCCGATC*T-3′;
(SEQ ID NO. 9; reverse primer)
5′-CAAGCAGAAGACGGCATACGAGAT[NNNNNNNNNN]GTGAC
TGGAGTTCAGACGTGTGCTCTTCCGATC*T-3′;
and
(SEQ ID NO. 10; forward primer)
5′-AATGATACGGCGACCACCGAGATCTACAC[NNNN]ACACTC
TTTCCCTACACGACGCTCTTCCGATC*T-3′;
(SEQ ID NO. 11; forward primer)
5′-AATGATACGGCGACCACCGAGATCTACAC[NNNNN]ACACT
CTTTCCCTACACGACGCTCTTCCGATC*T-3′;
(SEQ ID NO. 12; forward primer)
5′-AATGATACGGCGACCACCGAGATCTACAC[NNNNNN]ACAC
TCTTTCCCTACACGACGCTCTTCCGATC*T-3′;
(SEQ ID NO. 13; forward primer)
5′-AATGATACGGCGACCACCGAGATCTACAC[NNNNNNN]ACA
CTCTTTCCCTACACGACGCTCTTCCGATC*T-3′;
(SEQ ID NO. 14; forward primer)
5′-AATGATACGGCGACCACCGAGATCTACAC[NNNNNNNN]AC
ACTCTTTCCCTACACGACGCTCTTCCGATC*T-3′;
(SEQ ID NO. 15; forward primer)
5′-AATGATACGGCGACCACCGAGATCTACAC[NNNNNNNNN]A
CACTCTTTCCCTACACGACGCTCTTCCGATC*T-3′;
and
(SEQ ID NO. 16; forward primer)
5′-AATGATACGGCGACCACCGAGATCTACAC[NNNNNNNNNN]
ACACTCTTTCCCTACACGACGCTCTTCCGATC*T-3′,

wherein “[ ]” indicates the location of the sequence identifier (i.e., “barcode”) and “N” is any nucleic acid residue. The number of residues in each barcode is typically between 4 and 10. “C*T” indicates C and T linked by a phosphorothioate bond to prevent degradation.

In some embodiments, the forward primer has the reverse complement of the same barcode used in the reverse primer. In some embodiments, the barcodes used in the forward and reverse primers are completely independent.

Paired Sequence Tags

Paired sequence tags may be used. Paired-end sequencing provides the sequence of both ends of nucleic acid fragments and produces sequence data that can be aligned. Paired-end sequencing can detect repeating sequence elements, genomic rearrangements and gene fusions. Therefore, paired-end sequencing can improve the quality of the entire data set.

A single paired-end sequencing reads both forward and reverse template strands of each fragment. The reads can be combined to overlay long-range positional information, allowing for highly precise alignment of reads. In some embodiments, methods for paired-end DNA sequencing provide superior alignment across DNA regions containing repetitive sequences, and produce longer contigs for de novo sequencing by filling gaps in the consensus sequence. Paired-end DNA sequencing also detects rearrangements such as insertions, deletions, and inversions.

### V. Kits

The materials described above as well as other materials can be packaged together in any suitable combination as a kit useful for performing, or aiding in the performance of, the described method. It is useful if the kit components in a given kit are designed and adapted for use together in the described method. For example, described are kits for the size-specific enrichment and labelling of double stranded DNA fragments of genomic DNA according to the described methods.

Typically, kits include one or more sets of restriction endonuclease enzymes that cleave DNA upon recognition of specific nucleic acid sequences to yield blunt-ended DNA fragments. For example, kits for the sequencing of blunt-ended fragments of one or more specific DNA sequences from a genome include a multiplicity of different restriction endonuclease enzymes that cleave DNA to yield blunt-ended DNA fragments and universal sequence adapters, as well as buffers and other reagents necessary for digesting the nucleic acid samples, dA tailing the fragments and ligating the adapters to the digested fragments.

In certain embodiments, kits for genotyping by reduced representation sequencing can be customized to include a multiplicity of different restriction endonuclease enzymes to capture specific genomic DNA fragments.

The kits also can contain apparatus suitable for size-purification of the cleaved nucleic acid fragments complexes. Suitable apparatus can include SPRI beads and/or an affinity-binding column. The affinity binding column can contain SPRI beads, or another suitable substrate matrix. Preferably, the affinity-binding column facilitates simplified washing and handling of nucleic acid fragments, and allows automation of all or part of the method. Kits also can contain any other apparatus that provides a convenient means of washing away or otherwise separating undesirable reaction components from the target DNA fragments. An exemplary material for separation of DNA fragments on the basis of size is polyacrylamide, for example in the form of beads. Polyacrylamide beads suitable for separation of DNA species are available from multiple commercial sources (e.g., Biogel P100, available from BioRad catalogue number 150-4170). Therefore, kits can include a column containing Biogel P100.

Kits can contain substrates in any useful form, including thin films or membranes, beads, bottles, dishes, fibers, woven fibers, shaped polymers, particles and microparticles. In a preferred embodiment, kits contain substrates in the form of magnetic beads, for example, streptavidin coated paramagnetic beads (e.g., DYNABEADS(R)® M280 streptavidin, available from Thermo-Fisher Life Technologies catalogue number 112.05D; 112.06D or 602.10). Kits can also contain the buffers and reagents required to couple nucleic acids, wash the bound complexes and elute nucleic acids from the substrates. An exemplary buffer for coupling and washing includes 10 mM Tris-HCl (pH 7.5), 1 mM EDTA, 2M NaCl. Kits can also include other buffers and reagents that are commercially available from multiple sources (e.g., DYNABEADS® Kilobase BINDER™ kit, available from Thermo-Fisher Life Technologies catalogue number 60101). When magnetic beads are used, kits can also include suitable means for isolating the magnetic beads, such as a magnet.

In some embodiments, kits contain oligonucleotide primers that can hybridize to the sequence of the universal adapters. The oligonucleotide primers can optionally include sequence identifiers.

Kits can also contain instructions for performing the described methods, for example, instructions for enhanced reduced representation sequencing.

### VI. Systems

Described are systems useful for performing, or aiding in the performance of, the described method. Systems generally comprise combinations of articles of manufacture such as structures, machines, devices, and the like, and compositions, compounds, materials, and the like. Such combinations that are described or that are apparent from the disclosure are contemplated. For example, described and contemplated are systems comprising a device for processing nucleic acid samples according to the described methods, a device for size-selecting blunt-ended DNA fragments and a device for determining the nucleic acid sequence of the fragment, optionally including and assessing secondary characteristics, such as aligning and comparing the nucleic acid sequences. As another example, described and contemplated are systems including an automated device for fragmenting genomic nucleic acid samples and detecting the sequence, aligning the sequences and determining one or more parameters based on the alignment of the nucleic acid fragments.

Data Structures and Computer Control

Described are data structures used in, generated by, or generated from, the described method. Data structures generally are any form of data, information, and/or objects collected, organized, stored, and/or embodied in a composition or medium. For example, the nucleotide sequence of a blunt-ended, adapter ligated, labeled and enriched dsDNA fragment associated with a specific target sequence, or set of sequences stored in electronic form, such as in RAM or on a storage disk, is a type of data structure. The described method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be described herein.

The present invention will be further understood by reference to the following non-limiting examples.

### EXAMPLES

### Example 1

### Design and Implementation of Flexible and Scalable GBS Methods

### Materials and Methods

Preparation of the GBS Library and Sequencing

Leaf tissue was collected from the rice “Nipponbare,” maize inbreds B73 and “Country Gentleman”, the B73×CG F1 hybrid and 91 of its F2 progeny. DNA was extracted from leaf tissue as described (Chen and Dellaporta S L The Maize Handbook. Edited by Freeling M, Walbot V. New York: Springer-Verlag; 1994: 526-528). Approximately 500 ng of genomic DNA per sample was hybridized onto AMPure XL SPRI beads (AG3880, Beckman Coulter), cleaned as described in Broad Institute Protocol (Fisher, et al., Genome biology, 12(1):R1 (2011)), and digested with a 5-fold excess of restriction enzymes under manufacturer specified conditions for 2 hours. Genomic DNA from B73 and the Nipponbarre was digested with MlyI (R0610), AluI (R0137), RsaI (R0167), EcoRV (R0195), StuI (R0187), HaeIII (R0108), and HincII (R0103, New England Biolabs). For the F2 mapping population, RsaI and HincII were used to digest genomic DNA. Of the ninety-one F2 individuals in the B73×CG mapping population, eighty-nine were processed with RsaI, and ninety were processed with HincII.

Following digestion, a modified version of the standard ILLUMINA® library preparation was performed. The first modification was the omission of the end-repair step. As restriction enzymes compatible with this protocol produce blunt-end, 5′ phosphorylated DNA fragments, end-repairs is unnecessary. Further, end-repair would fix random, broken DNA fragments and add phosphate groups to their 5′ ends. This is undesirable as these ends would be highly random and result in irreproducible noise being added to the dataset. The second modification is the replacement of column-based cleanup with a Solid Phase Reversible Immobilization (SPRI) bead based methodology (Hawkins, et al., Nucleic acids research, 22(21):4543-4544 (1994)). as implemented by the Broad Institute (Fisher, et al., Genome biology, 12(1):R1 (2011)). In this method, double stranded DNA is immobilized on the paramagnetic beads held in place during buffer exchange, DNA size selection and cleanup steps. Wash, elution, and hybridization buffers were as described in the Broad Institute protocol. Following addition of beads, they are retained throughout the protocol until the post-adaptor ligation size selection step.

dA Tailing and Adaptor Ligation

Following digestion, samples were immobilized to the SPRI beads via addition of well-mixed beads at 3× concentration, then a wash was performed as described in the Broad Institute protocol. The end-repair was omitted for the reasons described above and dA tailing was performed. The addition of a 3′ adenine to DNA fragments ensures compatibility with standard adaptors having a single base overhang while preventing concatamer formation.

Methods for dA tailing of blunt-ended DNA fragments are known in the art. In some embodiments, were DNA fragments are coupled to a solid phase, such as beads, the DNA must first be eluted from the beads. For example, DNA fragments can be eluted with 40 μL of 10 mM Tris-HCl, then Exemplary methods include dA tailing using the Klenow Fragment (3′-5′ exo-) (M0212, New England Biolabs) per manufacturer's instructions. Following dA tailing, samples were once again washed per Broad Institute protocol. After elution into 40 μL of 10 mM Tris-HCl, ILLUMINA® Y-adaptors were ligated to DNA fragments using standard ILLUMINA® protocol with Broad Institute modifications for SPRI based library preparation (Fisher, et al., Genome biology, 12(1):R1(2011)). Ligation is done using the Quick T4 DNA ligase kit (M0202M, New England Biolabs) per manufacturer's protocol.

SPRI-Based Size Selection

A key advantage of SPRI based DNA manipulation is the ability to perform gel-free, in-solution size selection of DNA fragments. By varying the concentration of polyethylene glycol (PEG) in the hybridization buffer, DNA fragments below a certain size will fail to hybridize to the beads. As per the Broad institute protocol, 20% PEG, 2.5 M NaCl is added directly to the adapter ligation reaction at a final concentration of 0.3×, binding DNA fragments above 800 bp in size. The supernatant, which now contains DNA fragments below 800 bp in size is transferred to a new plate where 20% PEG 2.5 M NaCl is added at 1.2× volume to the supernatant, this time binding everything above approximately 100 bp to the SPRI beads. Supernatant is then discarded and beads are eluted with 30 μL of tris-HCl. Samples are now ready for PCR and addition of barcodes.

The SPRI methodology ultimately allows both column-free cleanup of samples and gel-free size selection, which makes it highly amenable to robust, large-scale multiplexing. Typically, SPRI beads represent a costly but worthwhile initial investment for large scale GBS, but for smaller experiments a more standard column/gel protocol may be optimal. Finally, it is worth noting that sizing by SPRI concentration does not produce hard cutoffs. Initially, ligation products were fractionate with lower limit of 200 bp, corresponding to approximately 80 bp DNA fragment plus adaptors and an upper limit of 800 bp, or 680 bp of gDNA. Following sequencing, significant DNA fragments below the expected size were observed and a variable upper size limit for DNA fragments that tended to be below 680 bp. Little or no adaptor dimer contamination was observed.

Barcoding and Multiplexing

Following size fractionation, to amplify the sequenceable portion of the library as well as add barcodes for sample identification post multiplexing, primers and a six cycle PCR using KAPA HiFi Master Mix (KK2101, Kapa Biosystems) were employed according to manufacturer's instructions. PCR conditions were 95° C. for 5 min followed by 6 cycles of 98° C. for 20 sec, 65° C. for 15 sec, and 72° C. for 30 sec. Finally, 72° C. for 1 min and 4° C. hold. Following barcoding, SPRI beads were added at 1.5× concentration, and samples were washed per Broad Institute protocol then pooled. Barcoding was performed using a dual-indexing system based on the TruSeq Dual Index Sequencing Primer Box that is further described in Lamble et al. (Lamble, et al., BMC biotechnology 13:104. (2013)). While the TruSeq Dual-Index Sequencing Primer Box (FC-121-1003, Illumina) offers compatibility with up to 96 libraries, much higher levels of multiplexing are possible with custom primers. Lamble et al. offer a list of 120 indices that meet the necessary requirements. Base primer sequences, which incorporate the barcodes. Primers with custom indices should be selected with input from the user's sequencing center to ensure compatibility with local protocols.

DNA Sequencing

The O. sativa and Z. mays digest sample libraries as well as the B73×CG HincII and RsaI mapping population libraries were sequenced as paired-end 75 bp reads on the ILLUMINA® HiSeq 2500 according to manufacturer's protocol. Image analysis and base calling was done using the ILLUMINA® version 1.8 pipeline with default parameters.

Computational Resources

Dataset analysis was performed on the Yale High Performance Computing Cluster. The YHPC clusters run a shared Linux environment with Perl ver 5.10.1, Python version 2.6.6, and Java version 1.7.0. Virtual restriction digest and associated data analysis In silico restriction digests were performed on the Z. mays B73 (v2) (Satiable, et al., Science, 326(5956):1112-1115(2009)) and O. sativa japonica Nipponbare 1.0 (Kawahara, et al., Rice (N Y), 6(1):4 (2013)) reference genomes for all tested enzymes using a custom Python script that employed a sliding window algorithm.

For MlyI, sites were identified on both the forward and reverse strands due to its non-palindromic recognition motif. Only reference positions that were a complete match to the recognition motif were recorded. The resulting digest map provided a framework for subsequent data analysis. Of interest for many downstream analyses were predicted sites, DNA fragments between proximal restriction sites. Predicted sites, due to their limited number compared to possible mispair (fragments generated from non-proximal restriction sites), singlet (fragments with only one end originating from a restriction site), and null (fragments with neither end originating from a restriction site), provided a useful control against sites with actual sequencing coverage for analyses of methylation, genic enrichment, GC bias, etc. Downstream analyses on the sequencing dataset and comparisons between aligned reads and predicted restriction sites were performed using custom Perl and Java scripts unless otherwise noted.

Read Alignment

Bowtie2 (parameters -N 1 -L 20 -D 20 -R 3 -I S,1,0.50) (Langmead, et al., Nature methods, 9(4):357-359 (2012)) was used to align Z. mays and O. sativa reads to the unmasked B73 reference genome and the Nipponbare O. sativa reference genome, respectively. These parameters were selected to maximize the probability of finding the correct alignment at the cost of increased runtime, which is especially important for the B73 genome given its high repetitive content.

### Results

Modifications to Existing GBS Methods

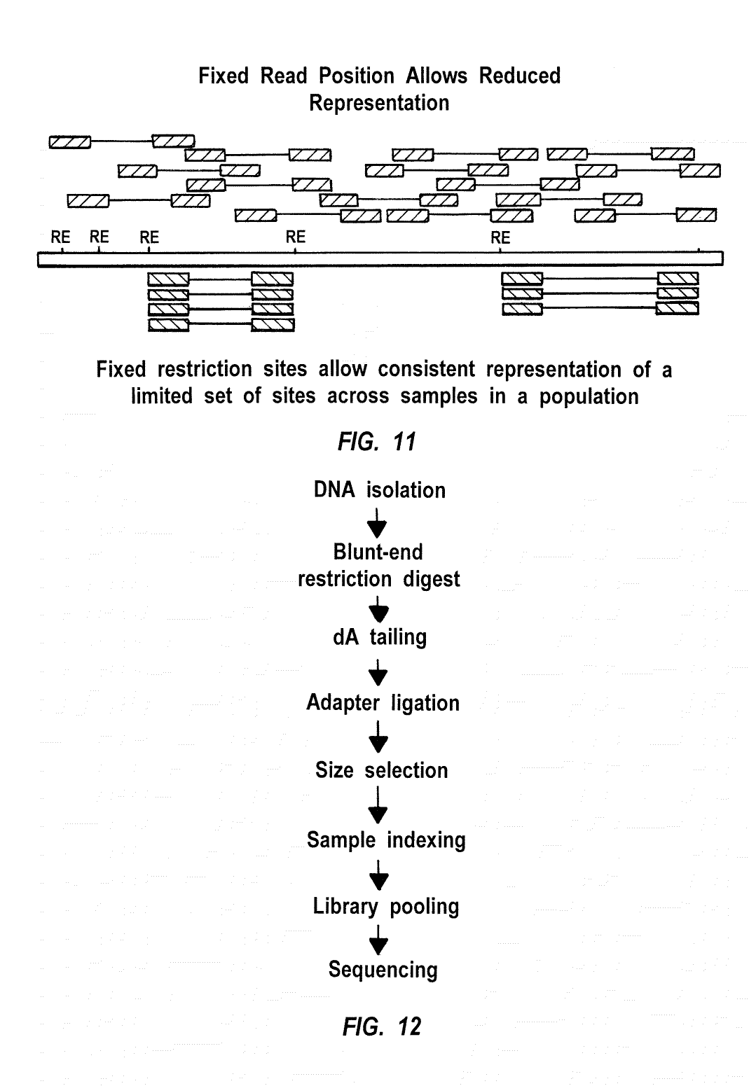

To improve the flexibility and scalability of GBS several modifications were incorporated into the protocol (FIG. 11). A key modification was that by choosing restriction enzymes that generated blunt ends fragments rather than ones with staggered ends, the custom enzyme-specific adaptors used in the original protocol (Elshire, et al., PloS one, 6(5):e19379 (2011)) could be replaced with standard ILLUMINA® Y-adaptors. This change removes the need for a costly end-repair step in the library preparation and enables the protocol to be compatible with a variety of enzymes. Supporting the switch to blunt-end enzymes and universal ILLUMINA® Y-adaptors, barcodes that were previously incorporated into custom adaptors were replaced with a primer-based method that adds dual indices, one to each end of an adaptor ligated DNA fragment, during a low-cycle PCR step (Lamble, et al., BMC biotechnology, 13:104 (2013)). Finally, a Solid Phase Reversible Immobilization (SPRI) (Hawkins, et al., Nucleic acids research, 22(21):4543-4544 (1994)) based library preparation allows for the entire protocol, including size-selection, to be done in microliter plates, without the need for gels or columns (Fisher, et al., Genome biology, 12(1):R1(2011)).

The results of these modifications were substantial reduction in cost, compatibility with a variety of blunt-end restriction enzymes, and a streamlined protocol that was adaptable to high throughput population genomic applications. The ability to choose restriction enzymes has several advantages as discussed later. To test the robustness of these changes to the GBS methods, eight blunt end restriction enzymes were surveyed on two different plant reference genomes Zea mays B73 (Schnable, et al., Science, 326(5956):1112-1115 (2009)) and Oryza sativa japonica Nipponbare (Kawahara et al., Rice (N Y), 6(1):4 (2013)). These genomes differ significantly in size, repetitive content, and methylated fraction. These eight multiplexed samples from each library were pooled and sequenced. Enzymes, motifs, and summary sequencing information are summarized in Table 1.

The methods improved both flexibility and scalability of GBS by removing any requirement for custom enzyme-specific barcoded adaptors. Specifically, restriction enzymes were chosen that created blunt-end fragments that required a single adenylation step for compatibility with standard ILLUMINA® Y-adaptors. DNA barcodes required for multiplexing samples were added to the universal adaptors during a low-cycle PCR step. This dual indexing system allows a great number of samples to be multiplexed during sequencing. For instance, with just twenty indexed forward and twenty indexed reverse primers as many as four hundred samples can be multiplexed on a single HiSeq 2500 lane. Finally, a bead-based in-solution library preparation protocol facilitates automation and allows for gel-free size selection. Over forty blunt-end enzymes compatible with this GBS protocol are commercially available. Eight enzymes that represented a variety of recognition motif lengths, sequence contents, and methylation sensitivities were selected to test the robustness of these new methods. This panel of enzymes was used to create GBS datasets from two reference genomes Z. mays B73 (Schnable, et al., Science, 326(5956):1112-1115 (2009)) and Oryza saliva japonica Nipponbare (Kawahara et al., Rice (N Y), 6(1):4 (2013)). Haploid genome length (approximately 2500 Mbp and 430 Mbp respectively), repeat content, methylation, and genic fraction differ considerably between the two genomes. In addition, a maize F2 population consisting of ninety-one individuals was created from two maize inbreds B73×Country Gentleman and genotyped by GBS using two enzymes, RsaI and HincII.

### Example 2

### Validation of Flexible and Scalable GBS Methods: Restriction Enzyme Selection

### Materials and Methods

Validation of Restriction Motif in Reads

A detailed assessment of the quality of data produced was performed. The first parameter tested was the quality of the sequenced fragments by confirming the appropriate restriction motif at the end of reads. All restriction enzymes, other than MlyI, tested in maize and rice had >80% and in most cases >90% of reads with the proper cut-site (Table 1). MlyI is a special case, as its non-palindromic recognition site is offset from its cleavage site, which results in the restriction motif being absent from 50% of the reads. Only 38.9% and 37.5% of the reads in maize and rice were observed with the proper MlyI motif, however.

TABLE 1
Enzyme summary statistics

| Enzyme | MlyI | AluI | RsaI | DraI | EcoRV | StuI | HaeIII | HincII |
|---|---|---|---|---|---|---|---|---|
| Recognition | GAGTC(N)_{5/} | AG/CT | GT/AC | TTT/AAA | GAT/ATC | AGG/CCT | GG/CC | GTY/RAC |
| Motif: | | | | | | | | |

Maize

| | | | | | | | | |
|---|---|---|---|---|---|---|---|---|
| Reads | 11,092,770 | 68,513,249 | 13,758,608 | 2,039,750 | 1,495,384 | 785,205 | 60,419,585 | 1,011,458 |
| (2 × 75 bp) | | | | | | | | |
| Fraction | 0.389 | 0.995 | 0.969 | 0.957 | 0.882 | 0.904 | 0.996 | 0.851 |
| reads with | | | | | | | | |
| correct motif | | | | | | | | |

Rice

| | | | | | | | | |
|---|---|---|---|---|---|---|---|---|
| Reads | 2,970,049 | 73,426,557 | 7,953,490 | 7,181,944 | 498,460 | 415,512 | 35,197,321 | 526,507 |
| (2 × 75 bp) | | | | | | | | |
| Fraction | 0.375 | 0.995 | 0.994 | 0.996 | 0.946 | 0.971 | 0.998 | 0.890 |
| reads with | | | | | | | | |
| correct motif | | | | | | | | |

### Results

In Silico Site Prediction

A key goal of this project was to both be able to predict which sites would be covered by sequencing reads and to understand the factors affecting sequencing coverage. Simply quantifying each individual restriction site as having reads aligning to it or not would fail to distinguish between restriction sites that would reliably generate sequencing reads and restriction sites that generated spurious reads from singular events.

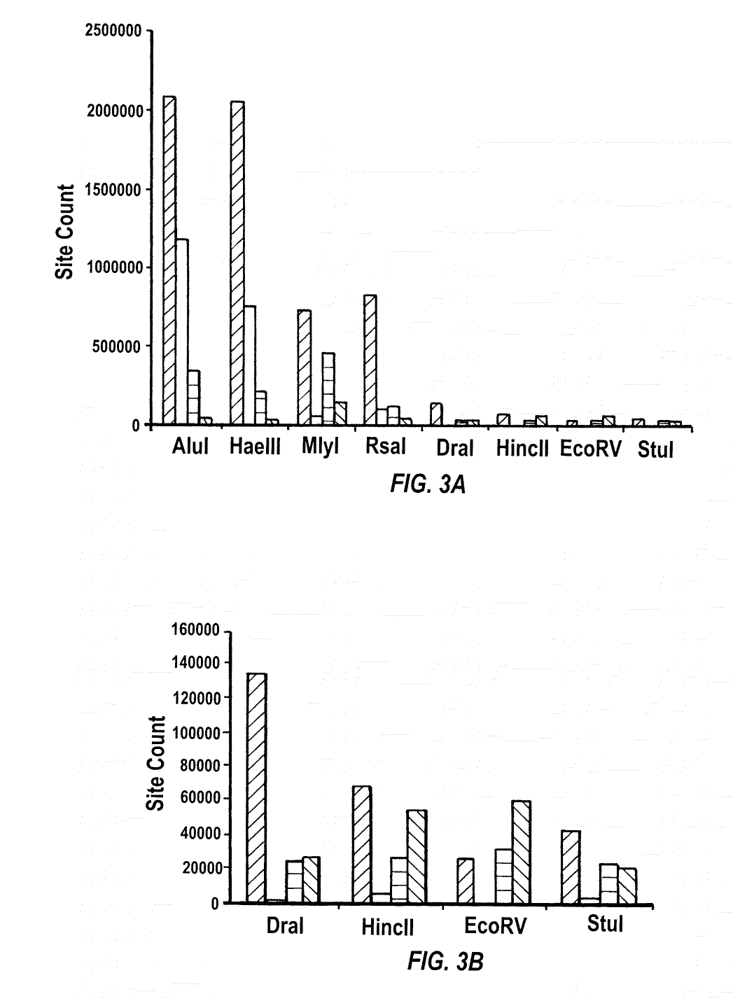

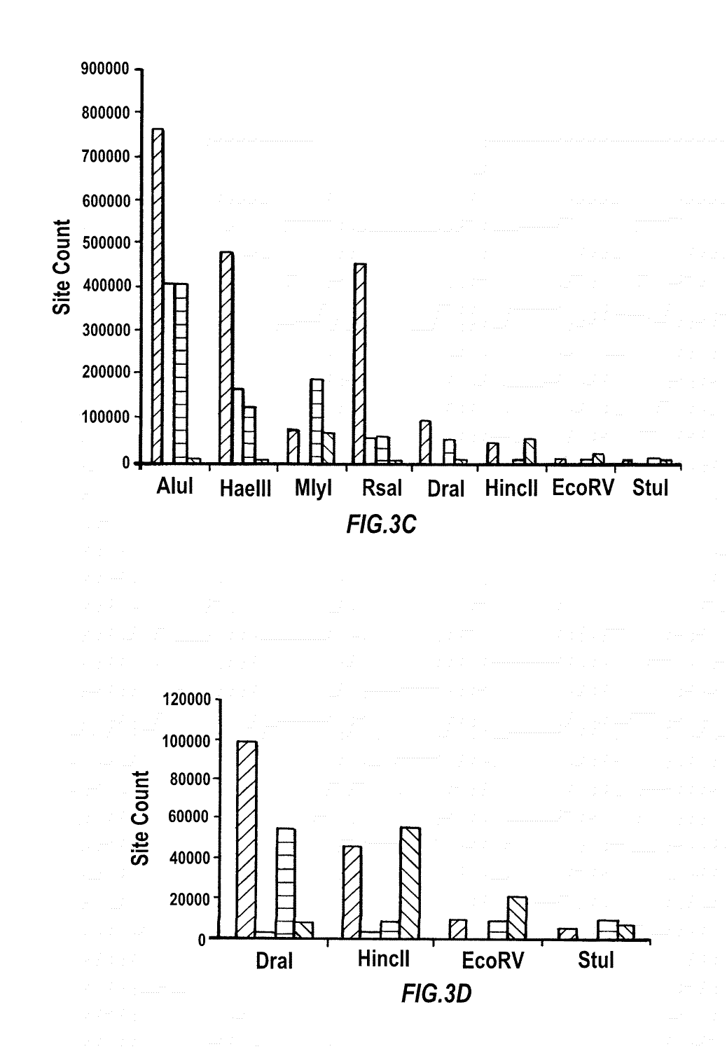

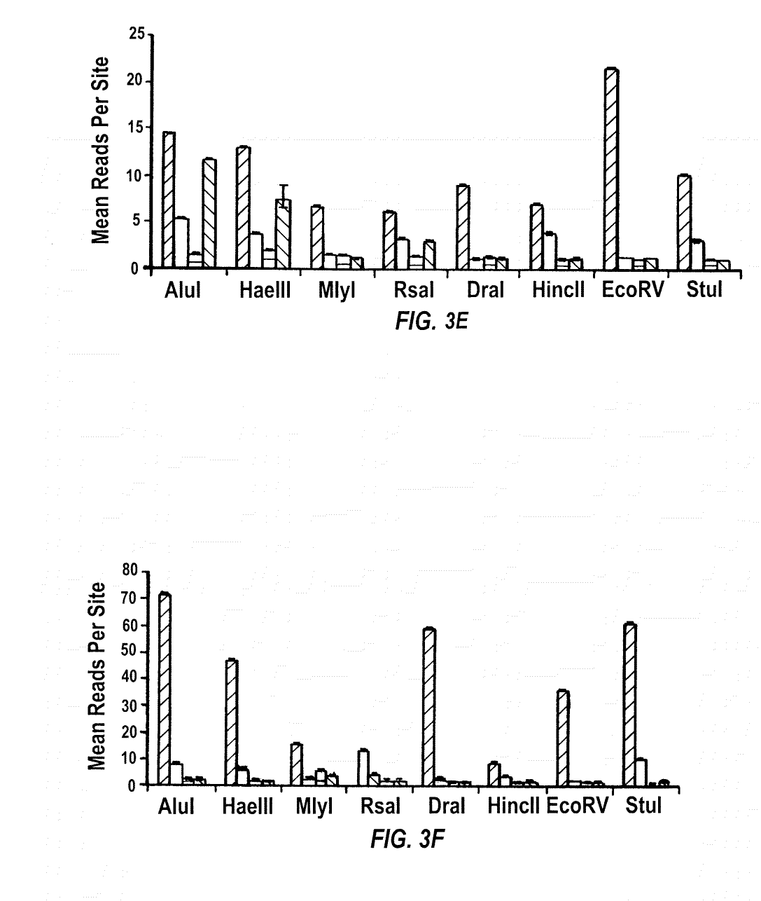

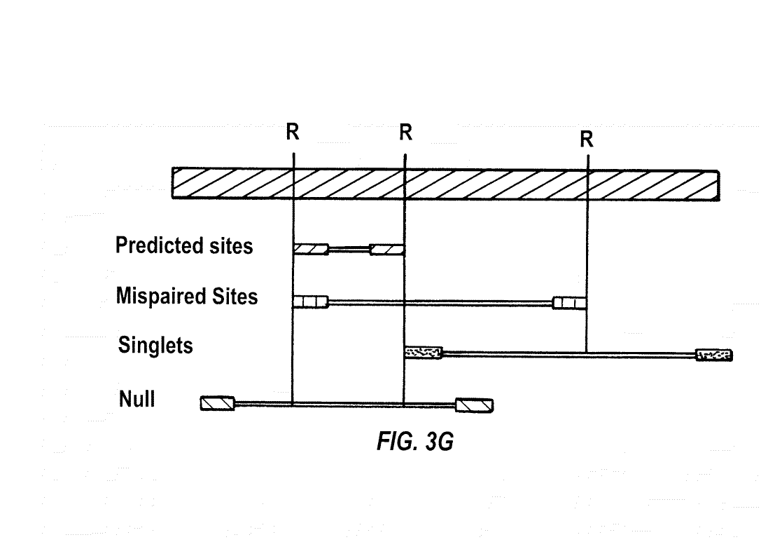

An example of a singular event would be a restriction site that would not normally be covered due to the distance between it and proximal restriction sites occurring sufficiently close to the random end of a DNA strand to produce a suitable fragment for sequencing. Instead, sites were classified into four categories based on restriction sites identified in silico and the alignments of both ends of paired-end reads (FIG. 3A-3G). An in silico digest of the maize and rice reference genomes identified predicted restriction sites for each enzyme. Following alignment to the genome, sequencing reads from each GBS dataset were categorized as “predicted” when the ends aligned to proximal restriction sites, “mis-paired” when the ends aligned to non-proximal restriction sites, “singlet” when only one end of a read aligned to a restriction site, and “null” when neither end of a read aligned to an in silico predicted restriction site. The number of each type of site with coverage (mapping quality (MQ)≧20) was determined for both (FIG. 3A, 3B) maize and (FIG. 3C, 3D) rice. The mean coverage per site type (MQ≧20) was also calculated in (FIG. 3E) maize and (FIG. 3F) rice for all enzymes.

“Predicted” sites were defined as reads originating from proximal restriction sites. Reads aligning to non-proximal restriction sites were designated as “mis-paired”. Paired reads with one end aligning to a restriction site and the other end aligning to no predicted restriction site were designated “singlets”. Reads that did not align to any predicted restriction site were identified as “null”. Only reads with a mapping quality (MQ) score≧20, or a 99% chance of correct alignment, were included for further analysis.

It was predicted that the vast majority of reads for all enzymes would originate from proximal restriction sites, which were designated as predicted sites. To test this, the alignments of actual reads (MQ≧20) were compared to in silico digest predictions of the maize and rice reference genomes. In maize, between 80.9% and 94.8% of all actual reads with MQ≧20 aligned to predicted sites, while in rice 71.3% to 94.8% of reads aligned to predicted sites (Table 2). In raw count of unique sites with sequencing coverage, predicted sites were the most common for all enzymes except EcoRV in maize (FIG. 3A, 3B) and HincII in rice (FIG. 3C, 3D). In terms of depth of coverage, predicted sites were the highest across all enzymes in both maize (FIG. 3E) and rice (FIG. 3F). The ultimate outcome of this analysis was the conclusion that proximal restriction sites are the origin of most sequenced reads. This provided a framework for the prediction of sequenced sites. This framework not only allowed us to predict what sites might be covered, but to compare the total set of predicted sites to the subset of sites with sequencing coverage to discover factors that influence site coverage.

Effect of Fragment Size on Coverage

DNA fragment size is a major factor affecting coverage in both maize and rice. The largest proportion of covered predicted sites in maize and rice occurs between 100 and 200 bp in all enzymes. For some enzymes, coverage of predicted sites extends outwards to 400 bp or more, but all enzymes show a reduction in the fraction of predicted sites with sequencing coverage after 400 bp. Therefore, the anchoring of reads to restriction sites and the bias in sequenced fragment sizes were two sources for reduced representation in genome coverage in GBS datasets. Further, depth of sequencing

TABLE 2
Predicted site counts and coverage

| | | | | | | | |
|---|---|---|---|---|---|---|---|
| MlyI | AluI | RsaI | DraI | EcoRV | StuI | HaeIII | HincII |

Maize

| | | | | | | | | |
|---|---|---|---|---|---|---|---|---|
| Total | 3,326,697 | 8,886,974 | 4,870,173 | 894,567 | 427,268 | 515,556 | 7,667,926 | 1,376,427 |
| predicted | | | | | | | | |
| sites | | | | | | | | |
| Predicted | 1,898,039 | 5,083,913 | 3,105,315 | 320,985 | 71,742 | 134,944 | 3,823,749 | 583,683 |
| sites | | | | | | | | |
| 100-1000 | | | | | | | | |
| bp | | | | | | | | |
| Predicted | 1,010,023 | 3,701,470 | 1,815,078 | 175,669 | 23,107 | 69,468 | 2,690,200 | 246,190 |
| sites | | | | | | | | |
| 100-400 | | | | | | | | |
| bp | | | | | | | | |
| Predicted | 391,160 | 1,936,120 | 746,075 | 78,672 | 10,382 | 21,045 | 1,436,751 | 103,861 |
| sites | | | | | | | | |
| 100-200 | | | | | | | | |
| bp | | | | | | | | |
| Total | 0.2226 | 0.2350 | 0.1705 | 0.1514 | 0.0604 | 0.0818 | 0.2684 | 0.0504 |
| predicted | | | | | | | | |
| sites | | | | | | | | |
| covered* | | | | | | | | |
| Covered | 0.3529 | 0.3465 | 0.2301 | 0.3701 | 0.3365 | 0.2986 | 0.4550 | 0.1068 |
| sites | | | | | | | | |
| 100-1000 | | | | | | | | |
| bp* | | | | | | | | |
| Covered | 0.5756 | 0.4721 | 0.3877 | 0.6503 | 0.7259 | 0.4126 | 0.6296 | 0.2439 |
| sites | | | | | | | | |
| 100-400 | | | | | | | | |
| bp* | | | | | | | | |
| Covered | 0.5986 | 0.5589 | 0.5524 | 0.7354 | 0.6872 | 0.4372 | 0.6746 | 0.2884 |
| sites | | | | | | | | |
| 100-200 | | | | | | | | |
| bp* | | | | | | | | |
| Fraction | 0.8363 | 0.8090 | 0.9000 | 0.9485 | 0.8373 | 0.8810 | 0.8874 | 0.8147 |
| of MQ | | | | | | | | |
| 20 reads | | | | | | | | |
| aligning to | | | | | | | | |
| predicted | | | | | | | | |
| sites | | | | | | | | |

Rice

| | | | | | | | | |
|---|---|---|---|---|---|---|---|---|
| Total | 371,222 | 1,486,508 | 1,037,100 | 301,435 | 78,181 | 60,707 | 1,204,615 | 260,304 |
| predicted | | | | | | | | |
| sites | | | | | | | | |
| Predicted | 188,264 | 875,739 | 641,398 | 128,513 | 14,728 | 10,684 | 565,466 | 111,036 |
| sites | | | | | | | | |
| 100-1000 | | | | | | | | |
| bp | | | | | | | | |
| Predicted | 90,536 | 623,512 | 408,979 | 70,456 | 6,146 | 4,093 | 371,004 | 49,065 |
| sites | | | | | | | | |
| 100-400 | | | | | | | | |
| bp | | | | | | | | |
| Predicted | 37,870 | 308,092 | 183,142 | 32,571 | 2,145 | 1,175 | 188,652 | 19,823 |
| sites | | | | | | | | |
| 100-200 | | | | | | | | |
| bp | | | | | | | | |
| Total | 0.2150 | 0.5132 | 0.4420 | 0.3281 | 0.1224 | 0.0867 | 0.4048 | 0.1757 |
| predicted | | | | | | | | |
| sites | | | | | | | | |
| covered* | | | | | | | | |
| Covered | 0.3578 | 0.7423 | 0.6226 | 0.6715 | 0.6005 | 0.4536 | 0.7235 | 0.3819 |
| sites | | | | | | | | |
| 100-1000 | | | | | | | | |
| bp* | | | | | | | | |
| Covered | 0.7026 | 0.8692 | 0.8513 | 0.9136 | 0.8967 | 0.6426 | 0.8732 | 0.5978 |
| sites | | | | | | | | |
| 100-400 | | | | | | | | |
| bp* | | | | | | | | |
| Covered | 0.8202 | 0.8684 | 0.8575 | 0.9062 | 0.9152 | 0.7668 | 0.8606 | 0.5681 |
| sites | | | | | | | | |
| 100-200 | | | | | | | | |
| bp* | | | | | | | | |
| Fraction | 0.7138 | 0.9224 | 0.9457 | 0.9854 | 0.9138 | 0.9361 | 0.9484 | 0.8298 |
| of MQ | | | | | | | | |
| 20 reads | | | | | | | | |
| aligning to | | | | | | | | |
| predicted | | | | | | | | |
| sites | | | | | | | | |

*A read alignment with MQ ≧20 is required for a site to be considered “covered”

coverage per site tends to be higher for smaller sites in both maize and rice, with the highest coverage occurring in sites between 100 and 200 bp. Covered, predicted sites>400 bp had the lowest coverage for all enzymes. Sites between 200 bp and 400 bp occupied an intermediate position. This observation suggests that while a complete coverage saturation of all possible sites may require an excessive number of reads, it is possible to achieve near saturation of sites within a limited size-spectrum at much lower depth of coverage.

Prediction of Coverage

The vast majority of reads for all enzymes align to proximal restriction sites. Further, these sites tend to be between 100 bp and 400 bp in size (FIGS. 3A-3G). This is likely a result of the size selection step during the library preparation and the bias of the ILLUMINA® sequencer towards smaller fragments.

Mispair sites tended to have lower coverage than predicted sites across all enzymes, but their >1× coverage values indicated that some mispair events were reproducibly covered. These may have been generated as a result of polymorphism or methylation disrupting a restriction site or the digest of a given site inactivating proximal ones.

Singlet sites, events where one end of a read aligned to a restriction site and the other end aligned to random DNA could also be generated from two potential sources. The first possibility is a polymorphism creating a restriction site that was not found in the reference. The other possibility is that the restriction site occurs near the random end of a DNA fragment. The latter is the most common case, as in most samples singlet sites were at or barely above 1× mean coverage, which suggests singular events.

Null sites occurred when neither end of a read aligned to a restriction site. For MlyI, DraI, HincII, EcoRV, and StuI in maize and all enzymes in rice save MlyI, these sites had a mean coverage near 1×, suggesting they were the result of random DNA fragments being sequenced. In AluI, HaeIII, and RsaI in maize, coverage was considerably above 1×, though the number of unique sites was small compared to the others. The likely reason for this is that some reads were misaligned to the same location in the genome multiple times. Several observations support this. First, as random fragments are generated from degradation, a consistent amount of these would be expected to be generated for each library as the amount of input DNA was equal between them. For enzymes that cut rarely and produce relatively few reads, such as Drat, HincII, EcoRV, and StuI, these would make up a larger overall proportion or the reads than for enzymes that cut frequently and generate large numbers of potential reads, such as AluI and HaeII. Second, misaligned reads represent a fraction of the total amount of reads generated and aligned. Thus, high coverage null sites are observed for enzymes that cut frequently and generate large datasets, such as AluI and HaeIII. Finally, null sites with high coverage generated by misalignments would be expected to be more common in maize, due to the highly repetitive and difficult nature of the genome, than in rice, which is much simpler to align reads to. This is also concordant with observations.

Finally, it is worth noting that all reads that are accurately aligned and whose alignments are observed across multiple samples in a population contribute to the value of a dataset, not just reads aligning to predicted sites. Mispair sites are the most common example of this, though singlet sites contribute as well. Given the likelihood that many null sites represent misalignments or broken DNA fragments, however, it may be advisable to filter these reads.

To further examine how well. GBS sequencing coverage was predicted, reads from two datasets, one produced by RsaI and the other by HincII, generated from a B73×CG F₂ population were realigned to the total set of predicted sites and to the set of predicted sites with sequencing coverage. As was expected from original datasets, the majority of coverage occurred between 100 and 400 bp. Predictability of coverage, as measured by the fraction of sites covered, improved when an F₂ sample's reads were aligned against sites covered in the pilot B73 experiment rather than the total set of predicted sites. In RsaI, this improvement was modest, with many samples only improving 540%. In HincII, however, the improvement was considerable. While only 30-40% of the total predicted sites were covered in each F₂ sample, up to 80-90% of the pilot-experiment sites were covered in the same F2 samples. The reasons for this are likely twofold. First, the identification of total predicted sites did not take into account the ability to unambiguously align reads to these sites. The use of a dataset based on predicted sites with sequencing coverage intrinsically did, as there was a MQ≧20 cutoff for sites. Second, the use of predicted sites with sequencing coverage by nature accounted for sites that were made inaccessible by methylation. The improvement in HincII data quality between the total and covered sites was likely due to this, as HincII is highly sensitive to methylation. Finally, though not as applicable in this case, pilot experiments account for differences between the target genome and the reference genome that cannot be identified in silico.

GC Content of Reads

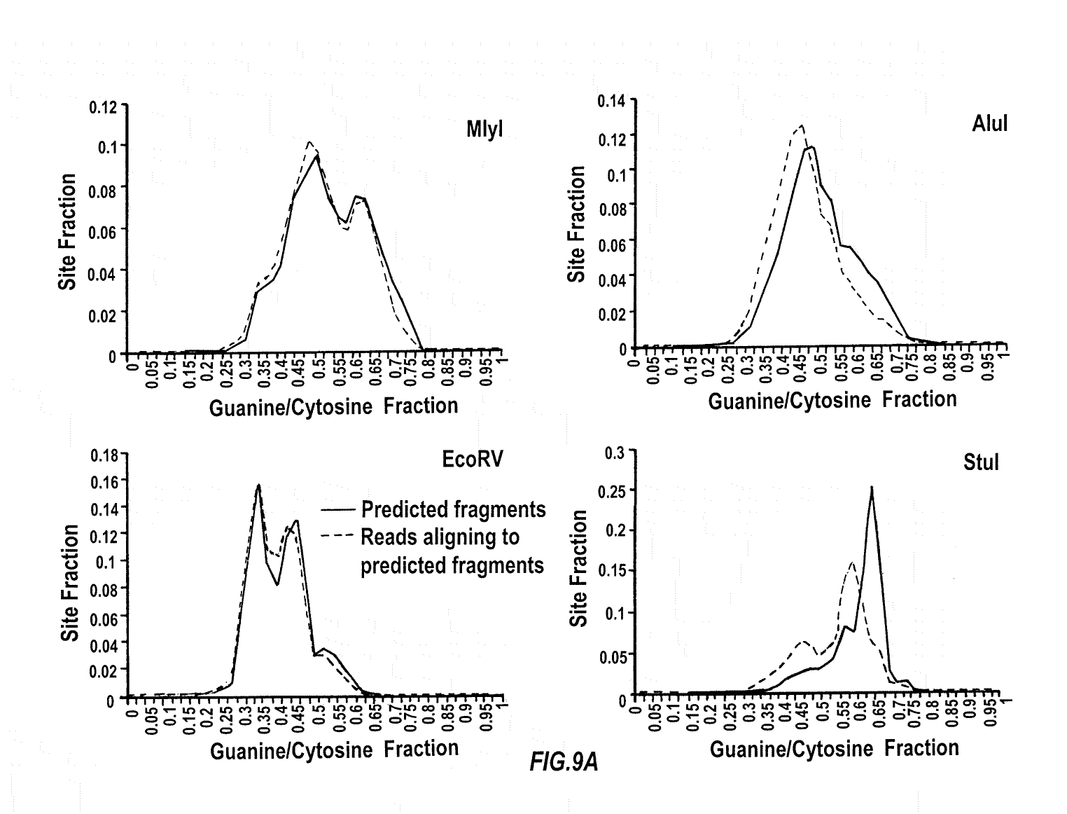

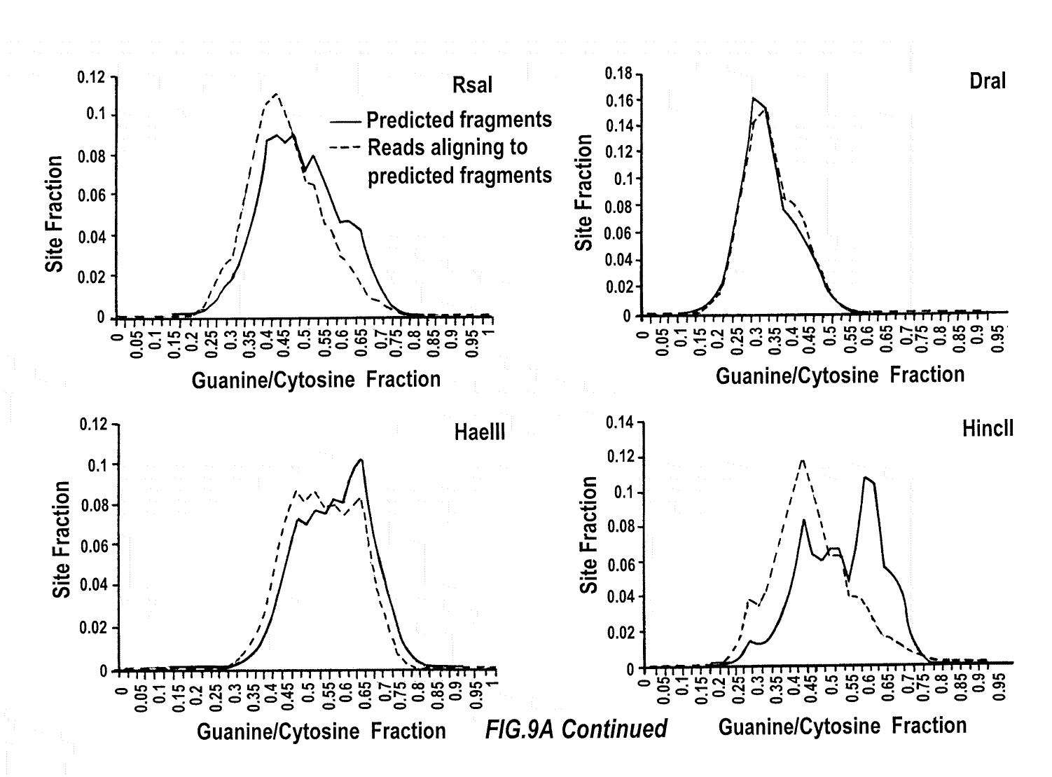

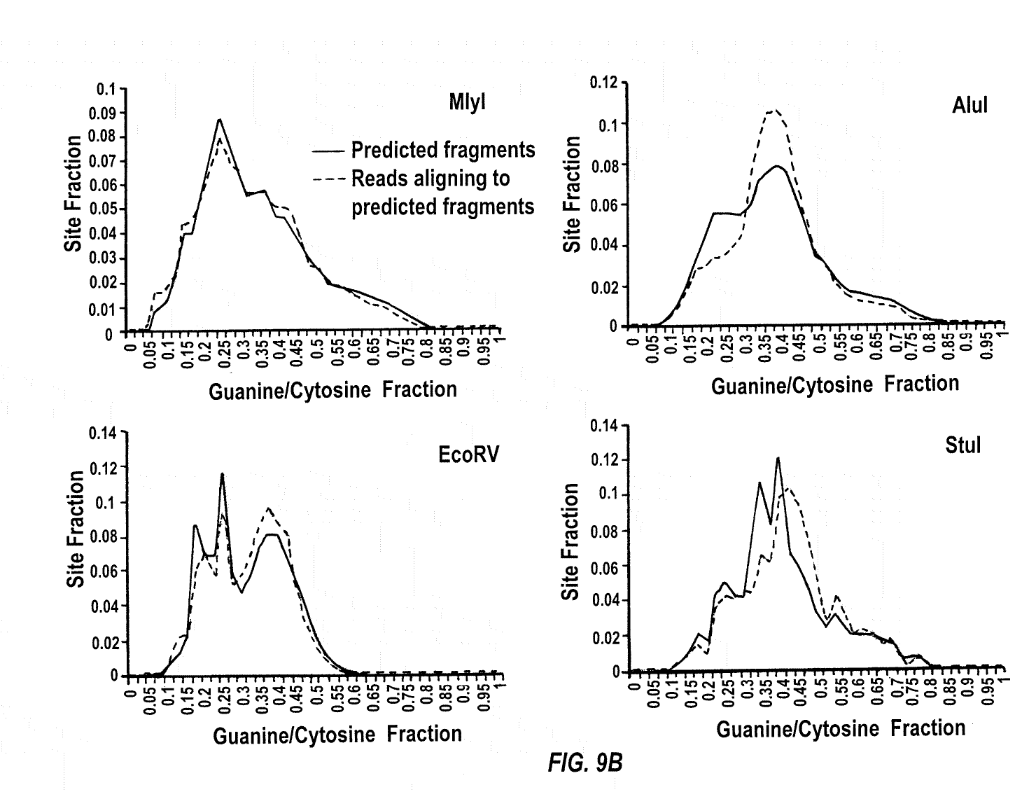

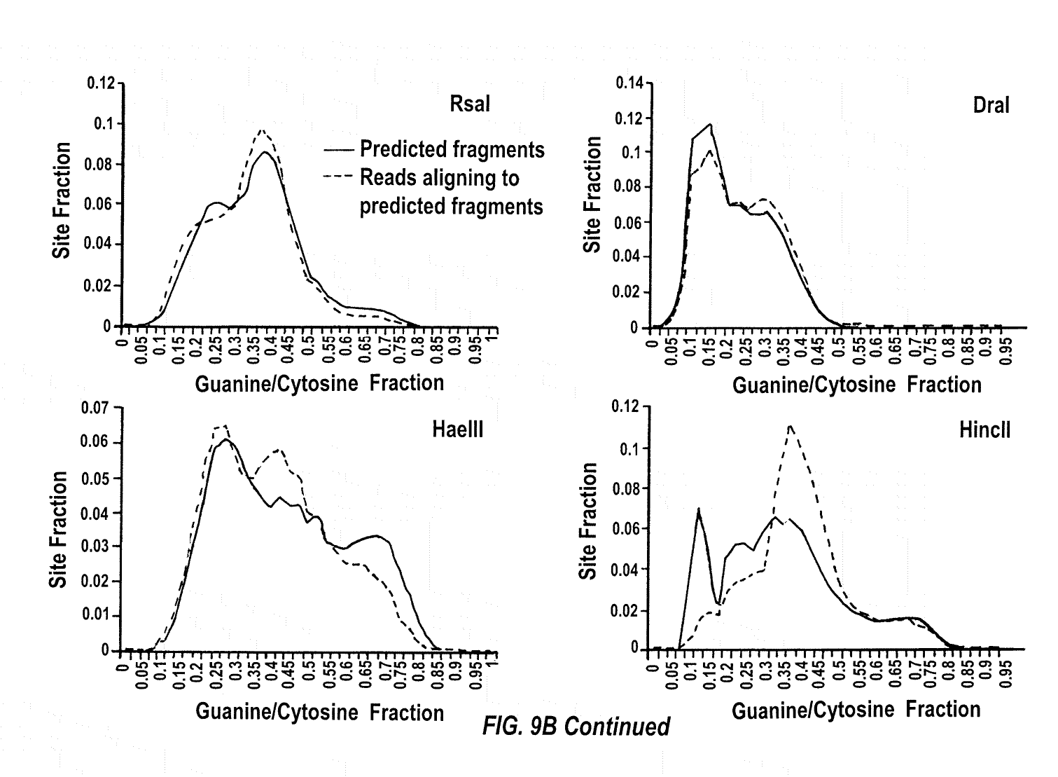

A source of coverage bias may be base composition of fragments due to poor amplification in the PCR step of library preparation. The protocol tried to minimize this bias by keeping the PCR cycles, necessary for indexing, to a minimum. In silico predicted sites, based on proximal restriction sites, were used to estimate bias in actual coverage due to the effect of base composition ratios. The GC ratios of all predicted sites between 100 and 200 bp were compared to the GC ratios of actual sequenced reads aligning to predicted sites between 100 and 200 bp in size for all tested enzymes in maize (FIG. 9A) and rice (FIG. 9B). Sites/reads were placed in 2.5% GC-content bins from 0 to 100% and predicted versus sequenced read distributions were compared via two-tailed paired t-test.

No bin showed a significant difference (p≦0.05) after correction for false discovery rate (Benjamini, et al., J Roy Stat Soc B Met, 57(1):289-300 (1995)). This suggests that the low number of cycles employed in barcoding and amplification (5-6) and the Kapa HiFi PCR reagents likely minimized any PCR bias in AT or GC rich regions.

Site Density

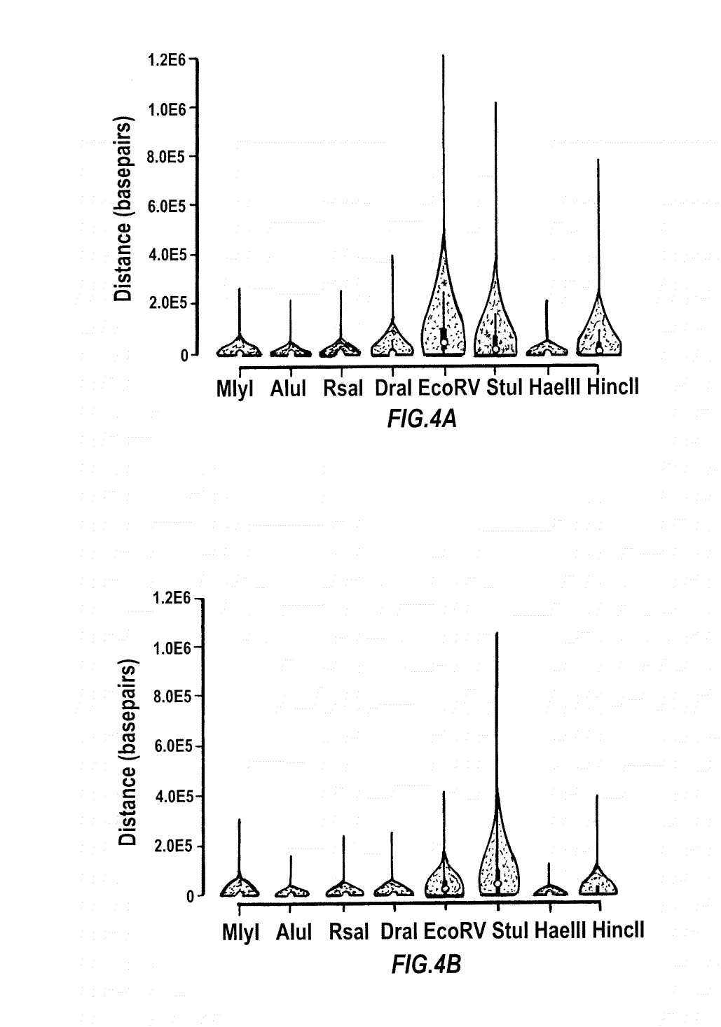

A factor important for the design of GBS experiments is the density of restriction site motifs found in a given genome. Site density will affect the ability to resolve recombination breakpoints and overall number of variants discovered. The distribution of distances between covered predicted sites with sequencing coverage was determined for all enzymes in maize (FIG. 4A) and rice (FIG. 4B). For all enzymes the shortest distance between predicted sites was 0 bp, indicating both upstream and downstream sequencing from a restriction site. In maize, AluI had the shortest mean distance between covered sites (811.2 bp±1739.2 bp (SD)) followed shortly after by HaeIII (811.8 bp±1928.8 bp (SD)). The longest mean distances between covered sites occurred in EcoRV (79430.0 bp±91774.1 bp (SD)) followed by StuI (48470.0 bp±66580.2 bp (SD)). In rice, AluI had the shortest mean distance between covered sites (251.7 bp±805.5), followed by HaeIII (516.0 hp±1234.1 bp (SD)). StuI had the longest mean distance (70400.0 bp±84923.3 bp (SD)) followed by EcoRV (38600.0 bp±45027.4 bp (SD)). The longest interval without a covered site observed in any organism was 1.2 Mbp (EcoRV, maize).

### Example 3

### Validation of Flexible and Scalable GBS Methods: Genic Enrichment and Methylation Sensitivity

### Materials and Methods

Assessment of Genic Enrichment

Genic enrichment was determined by comparing the total set of predicted sites and predicted sites with sequencing coverage to gene databases for maize and rice. These datasets give the positions of introns, exons, and untranslated sequences. For maize, the utilized dataset was the filtered, 5 b dataset (maizesequence.org) (Schnable, et al., Science, 326(5956):1112-1115 (2009)), which has transposases, pseudo-genes, contamination, and low confidence events. The rice dataset was the IRGSP 1.0 reference dataset, which includes intronic, exonic, an untranslated sequence (Kawahara, et al., Rice(N Y), 6(1):4 (2013)). This dataset is supported by FL-cDNAs, ESTs, and proteins.

Assessment of Methylation Sensitivity

Methylation sensitivity was determined by comparing nucleotide frequencies around the set of total, predicted restriction sites to nucleotide frequencies around predicted sites with sequencing coverage. Differences between predicted and covered datasets in guanine ratios 1-2 bases upstream and cytosine ratios 1-2 bases downstream of restriction motifs were potentially due to methylation, as plant methylation can occur at CpG and CpNpG motifs. Changes in other nucleotide ratios were used to measure variance between predicted and covered sites not caused by methylation. The total set of predicted versus covered nucleotide ratios was further divided into genic and non-genic groups based on annotated datasets (Kawahara, et al., Rice (N Y), 6(1):4 (2013); (Schnable, et al., Science, 326(5956):1112-1115 (2009)).

### Results

Coverage in Genic Regions

An important parameter in experimental design was the fraction of predicted sites with sequencing coverage in genic regions. Markers in genic regions are generally held to be more informative than non-genic markers as they are less repetitive and, for many studies, more likely to be in proximity of a trait-associated polymorphism. The genic fractions of all predicted sites and sites with sequencing coverage in genic regions were determined (Table 3).

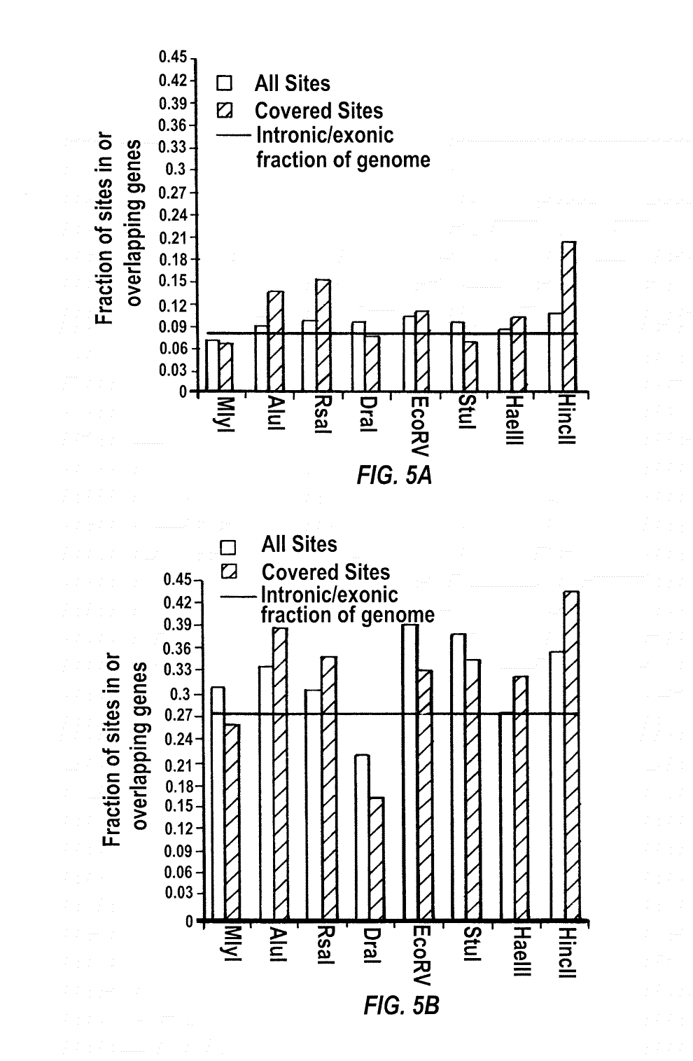

The fraction of predicted sites with aligned reads versus total predicted sites in genic regions was assessed. The maize and rice genomes were binned into 1 Mbp intervals, then within each bin the fraction of covered sites in genic regions was compared to the fraction of predicted sites in genic regions. Bins were then plotted based on the two ratios and the number of bins in a given point indicated via heat map to indicate the predicted values at which the covered and predicted genic fractions would be identical. Points plotted above the line represented bins with a greater fraction of sequenced sites in genic regions than predicted. Predicted genic site fraction varied from enzyme to enzyme, but in maize (FIG. 5A) the covered genic fraction for HincII (0.104 predicted, 0.203 covered), AluI, (0.087 predicted, 0.134 covered) and RsaI (0.095 predicted, 0.153 covered) were considerably higher than predicted. In rice (FIG. 5B), covered genic fractions tended to be closer to predicted genic fractions for all enzymes tested. To better understand the ratio of the total predicted and covered predicted genic fractions, termed genic enrichment, the maize and rice genomes were divided into 1 Mbp bins. The predicted versus covered genic ratio was plotted for each of these bins and graphed. While both the predicted and covered genic fractions did vary from bin to bin based, likely based on genic fraction within the bin itself, the relationship between the two was consistent for most enzymes.

Enzyme Methylation Sensitivity

One possible factor responsible for the enrichment of covered sites in genic regions relative to the predicted values for some enzymes is cytosine methylation sensitivity of the restriction enzyme. Repetitive DNA in plants tends to be methylated at CpG and CpNpG motifs. Digestion of repetitive gDNA by methylation sensitive enzymes may result in DNA fragments too large to sequence being generated, whereas non-methylated regions would produce a normal DNA size spectrum.

TABLE 3
Genic fractions of total and covered predicted sites

| | | | | | | | |
|---|---|---|---|---|---|---|---|
| MlyI | AluI | RsaI | DraI | EcoRV | StuI | HaeIII | HincI |

Maize

| | | | | | | | | |
|---|---|---|---|---|---|---|---|---|
| Fraction total predicted genic sites | 0.0668 | 0.0868 | 0.0957 | 0.0921 | 0.1021 | 0.0921 | 0.0836 | 0.1040 |
| Fraction predicted genic sites | 0.0621 | 0.0900 | 0.0982 | 0.0766 | 0.0820 | 0.0542 | 0.0823 | 0.0967 |
| 100-1000 bp | | | | | | | | |
| Fraction predicted genic sites | 0.0543 | 0.0908 | 0.1011 | 0.0624 | 0.0940 | 0.0428 | 0.0804 | 0.0953 |
| 100-400 bp | | | | | | | | |
| Fraction genic sites | 0.0538 | 0.0900 | 0.1068 | 0.0560 | 0.0780 | 0.0495 | 0.0804 | 0.0865 |
| 100-200 bp | | | | | | | | |
| Fraction total covered sites genic* | 0.0657 | 0.1368 | 0.1531 | 0.0768 | 0.1067 | 0.0669 | 0.1007 | 0.2031 |
| Fraction covered sites genic | 0.0660 | 0.1321 | 0.1488 | 0.0757 | 0.1050 | 0.0646 | 0.0967 | 0.1993 |
| 100-1000 bp* | | | | | | | | |
| Fraction covered sites genic | 0.0638 | 0.1317 | 0.1484 | 0.0742 | 0.1093 | 0.0664 | 0.0964 | 0.1958 |
| 100-400 bp* | | | | | | | | |
| Fraction covered sites genic | 0.0630 | 0.1321 | 0.1503 | 0.0661 | 0.0928 | 0.0763 | 0.0961 | 0.1737 |
| 100-200 bp* | | | | | | | | |

Rice

| | | | | | | | | |
|---|---|---|---|---|---|---|---|---|
| Fraction total predicted genic sites | 0.3102 | 0.3342 | 0.3070 | 0.2196 | 0.3917 | 0.3794 | 0.2756 | 0.3561 |
| Fraction predicted genic sites | 0.2793 | 0.3367 | 0.3261 | 0.1721 | 0.3124 | 0.2261 | 0.2977 | 0.3021 |
| 100-1000 bp | | | | | | | | |
| Fractions predicted genic sites | 0.2471 | 0.3442 | 0.3112 | 0.1439 | 0.2790 | 0.2084 | 0.2910 | 0.3021 |
| 100-400 bp | | | | | | | | |
| Fraction genic sites | 0.2244 | 0.3468 | 0.3041 | 0.1328 | 0.3016 | 0.2417 | 0.2929 | 0.2867 |
| 100-200 bp | | | | | | | | |
| Fraction total covered sites genic* | 0.2588 | 0.3871 | 0.3487 | 0.1619 | 0.3321 | 0.3455 | 0.3240 | 0.4336 |
| Fraction covered sites genic | 0.2625 | 0.3837 | 0.3502 | 0.1646 | 0.3334 | 0.3413 | 0.3222 | 0.4178 |
| 100-1000 bp* | | | | | | | | |
| Fraction covered sites genic | 0.2587 | 0.3796 | 0.3435 | 0.1504 | 0.2970 | 0.2958 | 0.3180 | 0.4178 |
| 100-400 bp* | | | | | | | | |
| Fraction covered sites genic | 0.2423 | 0.3825 | 0.3366 | 0.1395 | 0.3158 | 0.3019 | 0.3241 | 0.4060 |
| 100-200 bp* | | | | | | | | |

*A read alignment with MQ ≧20 is required for a site to be considered “covered”

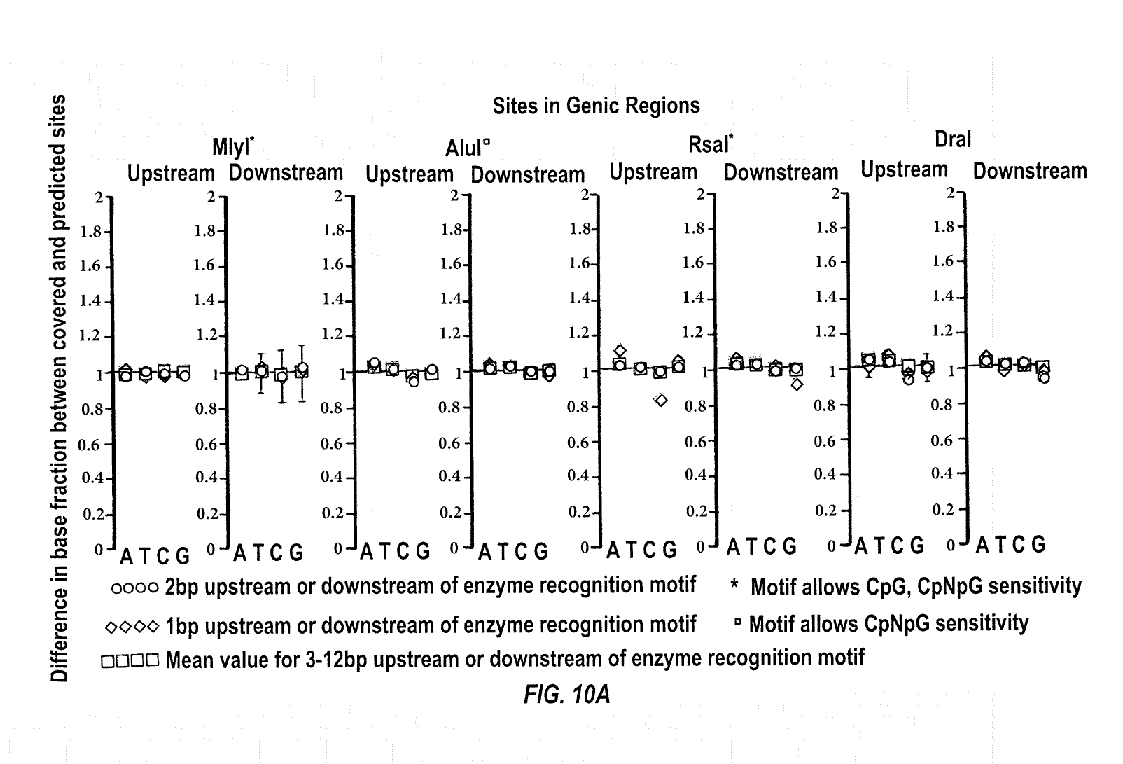

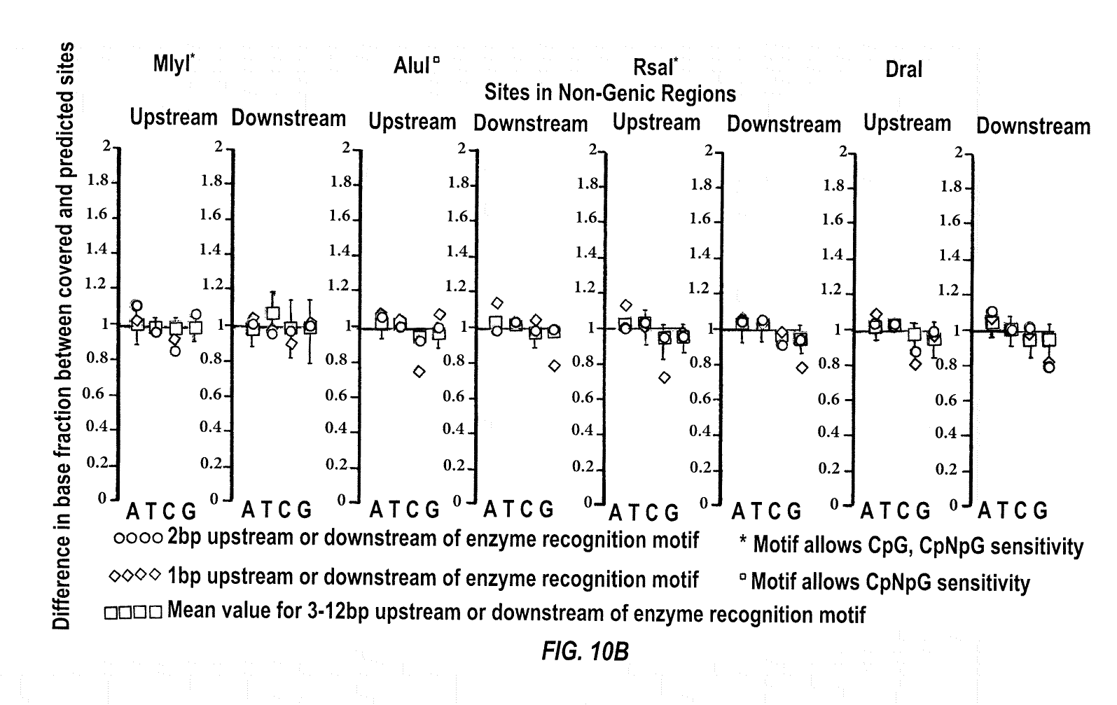

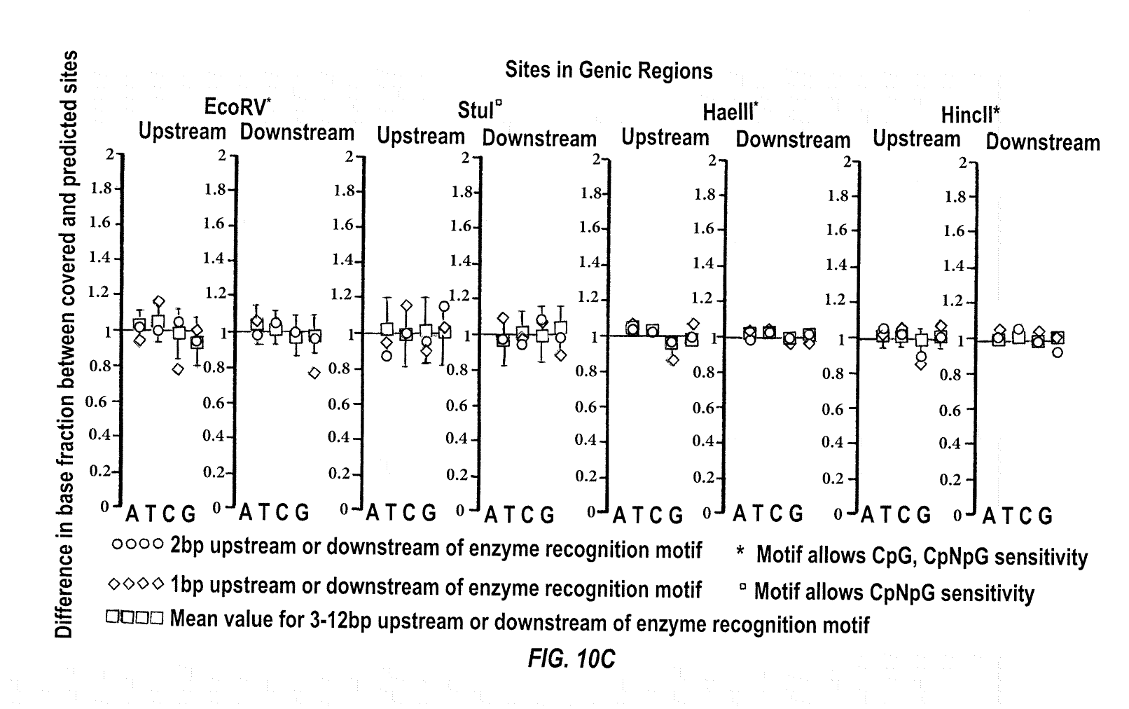

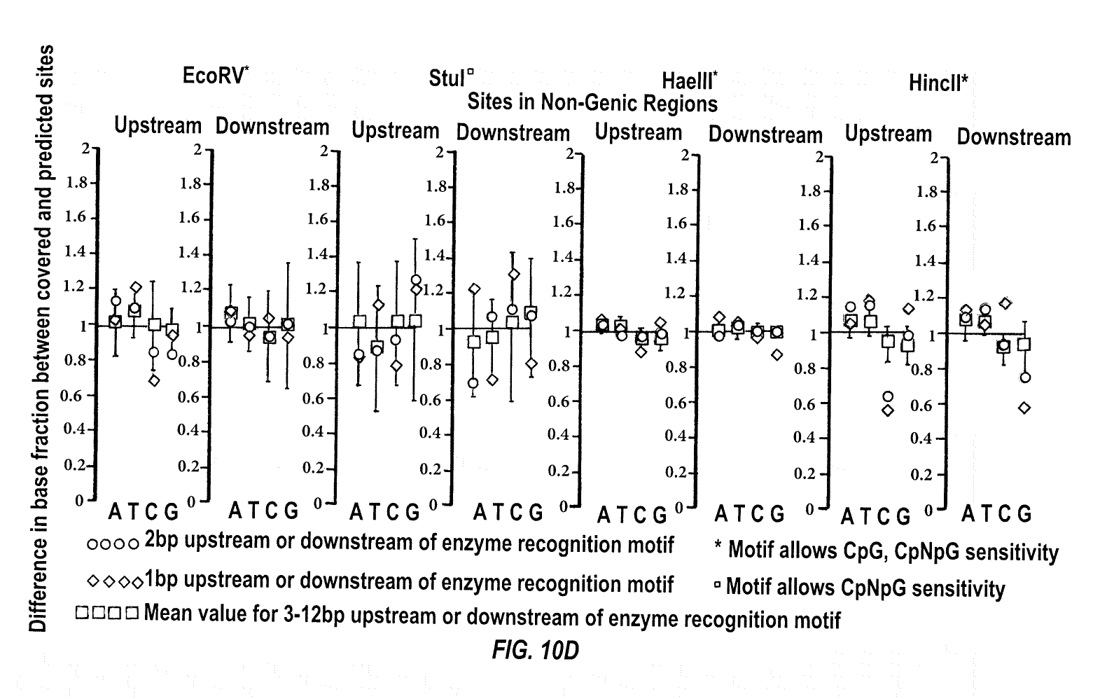

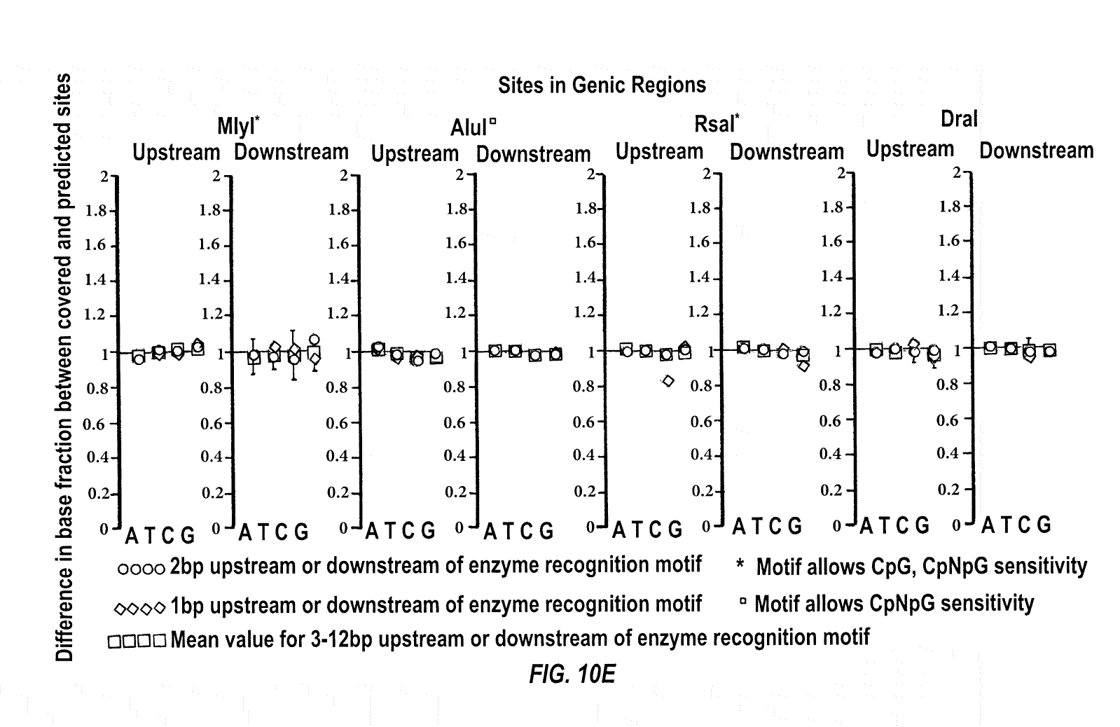

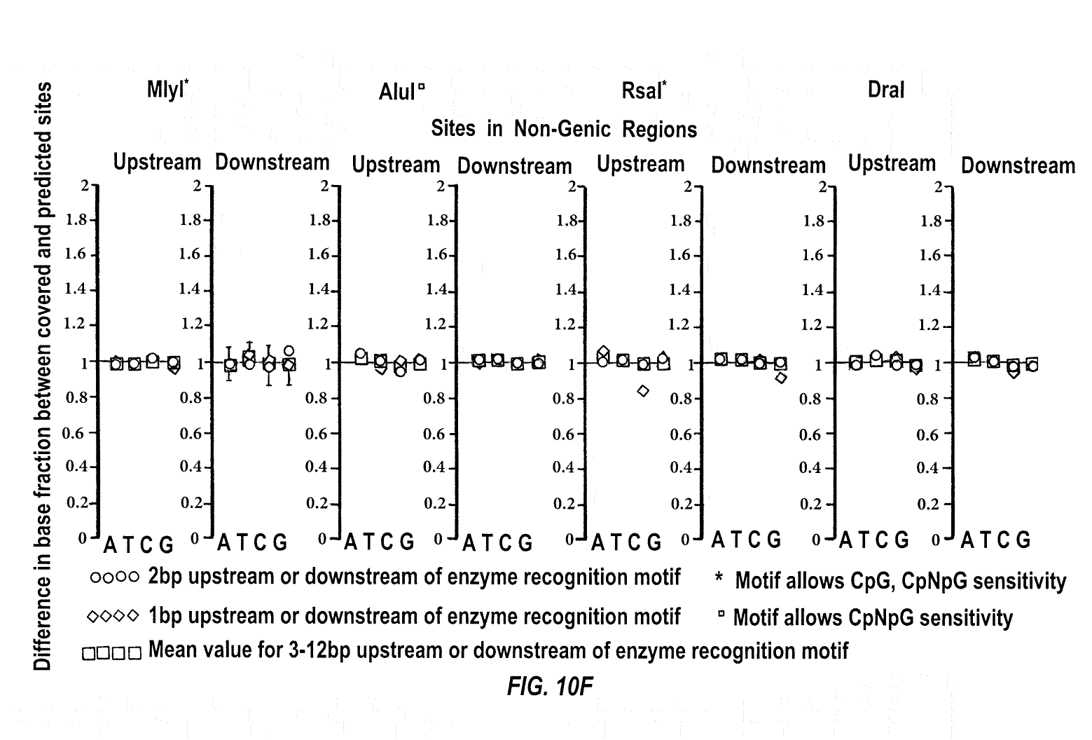

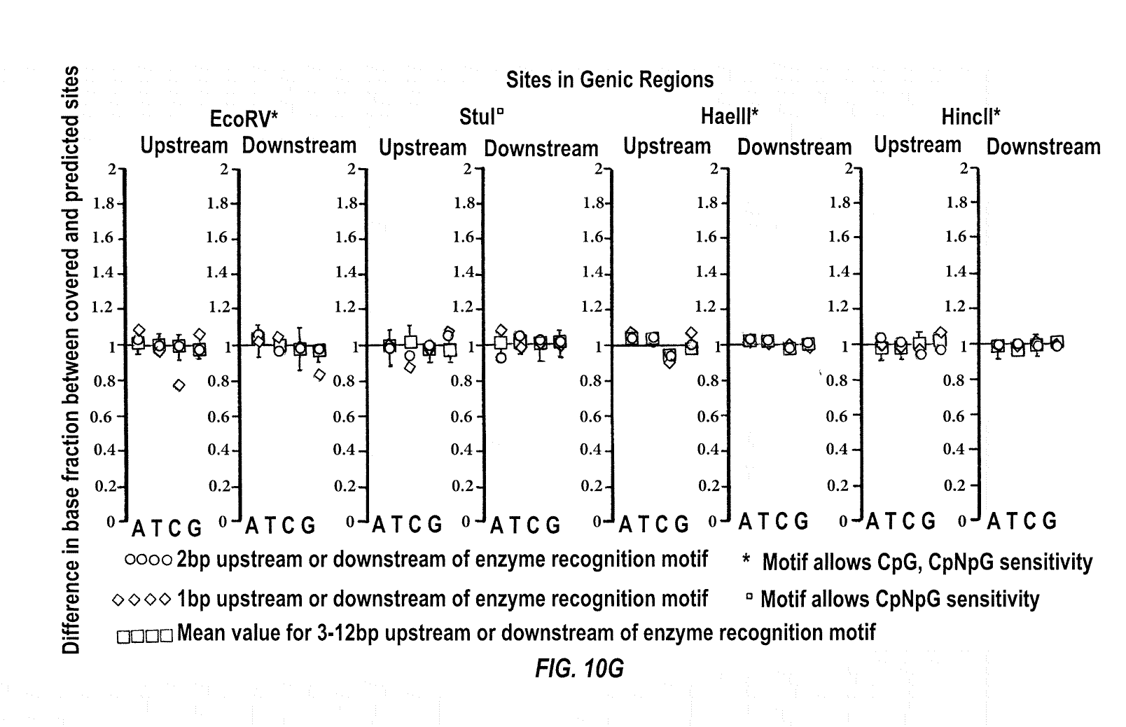

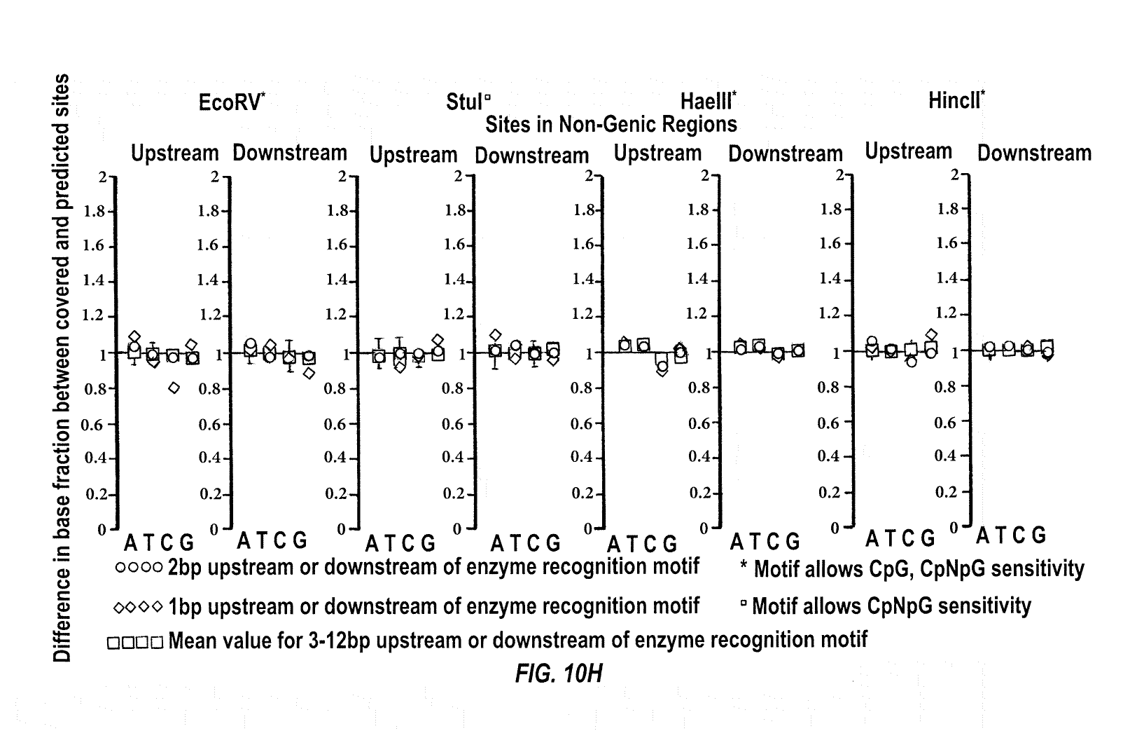

To assess the contribution of cytosine methylation to genic enrichment, the nucleotide ratios flanking the motifs of restriction sites were compared in predicted sites with aligned reads for a given enzyme versus the total set of predicted sites. Sites were further broken up into ones overlapping introns and exons and sites in non-genic regions, as repetitive, intergenic regions are often methylated (FIGS. 10A-10H). This analysis indicated that in maize several enzymes, namely HincII, RsaI, and AluI show considerable reductions in guanine one to two by upstream and cytosine one to two by downstream of restriction motifs. Further, this difference is more pronounced in non-genic than in genic regions.

(1) Maize

In maize, HincII was sensitive to both CpNpG and CpG methylation. HincII had the largest genome-wide decrease between predicted and covered upstream cytosine (from 0.227 to 0.123) and downstream guanine (from 0.225 to 0.128) ratios. Further, it had greatest increase in covered versus predicted sites in genic regions of all tested enzymes (10.04% sites predicted to be in genes, 20.03% covered sites in genes, 1.95-fold increase). RsaI, showed clear sensitivity to CpG methylation but was much less sensitive to CpNpG methylation. RsaI showed a 1.45-fold enrichment in predicted sites with sequencing coverage in genic regions versus all predicted sites (9.57% predicted, 15.31% actual). Interestingly, the enzyme with the third highest increase covered genic fraction relative to predicted (1.58-fold) was AluI, which, due to its recognition motif of AGCT, was only sensitive to CpNpG methylation (FIGS. 10A, 10B, 10C, 10D).

(2) Rice

In the less repetitive rice genome, predicted versus covered nucleotide ratios were similar for most enzymes, and differences between covered and predicted sites for a given enzyme in rice were smaller than in maize (FIGS. 10E, 10F, 10G, 10H). In rice, HincII was the enzyme with the largest difference in G/C ratios between predicted and covered sites. The cytosine ratio 1 bp upstream of the HincII motif decreased from 0.240 to 0.189 and the guanine ratio downstream decreased from 0.239 to 0.192 between total and covered predicted sites. That G/C ratios would be closer between covered and predicted sites in rice than maize was expected, as no enzyme in rice had a covered sites genic fraction>25% that of predicted sites. These results indicated that benefits conferred from methylation sensitive enzymes are genome dependent.

It is worth noting that, while different enzymes showed different degrees of methylation sensitivity in this study, this may be a product of the genomes tested more than an intrinsic property of the enzymes themselves. If an enzyme's recognition motif predisposes it to cut more often in a repetitive region, it may appear more methylation sensitive than one whose recognition site biases it away from these regions.

### Example 4

### Validation of Flexible and Scalable GBS Methods: Mapping Quality

### Materials and Methods

Variant Calling

Variants were called from aligned reads using Samtools mpileup (Li, et al. Bioinformatics, 25(16):2078-2079 (2009)). Variants retained in the final B73×CG dataset were required to have Phred≧30, MQ≧30, homozygous, opposite states in the parentals, ≧2× coverage in 20 F2 samples, heterozygosity≧0.2 and ≦0.8 in F2, and mean r2 correlation≧0.3 five variants upstream or downstream (Additional file 10).

Data Imputation

Missing variant states were not directly imputed; instead, regions were classified as B73 homozygous, heterozygous, or CG homozygous. For this, variants were first phased by parental states, then a most likely state (B73 homozygous, CG homozygous, or heterozygous) was determined in 5 Mbp sliding window across the genome using a least squares based method. The method can be described using the equation:

$$S = {\sum }_{i = 1}^{n} {r}_{i}^{2}$$

Where S is the sum of residuals, and r is the residual defined by the equation:

rᵢ=gᵢ−mᵢ

Where gᵢ is the window genotype and mᵢ is the individual marker's genotype. The three possible marker genotypes, homozygous B73, heterozygous, and homozygous CG were assigned values of 0, 1, and 2 respectively. Each possible “overall” genotype is assigned a value using the same system, and each of the three possible genotypes is tested against the set of markers. The genotype with the lowest sum of squared residuals is assigned to the window. In windows where less than ten total variants existed, variant states in proximal windows were included. Recombination breakpoints were resolved by first identifying proximal bins with differing calls. A five marker sliding window was then moved across the two proximal bins in a forward and reverse direction and a genotype call was obtained at each point. When the window transitioned from the first bin's genotype to the second's and vice versa, the point was recorded. Finally, the mean value of the two transition points was used as the point of recombination. This method was employed to resolve heterozygous regions in GBS data in spite of the high rate of missing and erroneous data, especially false homozygous calls resulting from low coverage of heterozygous SNPs.

Trait Mapping

Two traits (y1 and su1) with previously mapped genetic positions segregated within the B73×CG F2 population. Genotypes of F2 individuals for both traits were determined based on the F3 endosperm phenotypes. Trait mapping was performed on pre and post-imputation datasets of filtered markers using a custom script utilizing the apache commons (commons.apache.org/math) implementation of the One-Way ANOVA test.

Resampling of RsaI and HincII Datasets

One RsaI sample (F2-44) and one HincII sample (F2-23) were selected from the B73×CG F2 population and subsets of reads were randomly subsampled from each dataset. For RsaI, reads were subsampled in 100,000 read intervals from 100,000 reads to 2,000,000 reads, in 200,000 read intervals from 2,000,000 reads to 3,000,000 reads, and in 500,000 read intervals from 3,000,000 reads to 7,000,000 reads. The original sample had 15,389,878 2×75 bp reads. For HincII, reads were subsampled at 30,000, 40,000, and 50,000 reads and from 100,000 to 3,000,000 reads in 100,000 read intervals. The original sample (F2-23), had 3,968,544 2×75 bp reads. Sub-samplings were done to cover the range of diminishing returns for additional markers. The lowest value for each sample was determined by the point at which imputation would fail due to too few markers. Each subset was independently aligned to the genome, variant calling and filtering applied, and finally genotypes were imputed. To evaluate the subsamples the number of shared, post-filter markers was compared between the original sample and the subsets. In addition, the fraction of the genome that shared the same call between the subset and the original was determined.

Mapping Quality

A major source of data loss in GBS and many other next generation sequencing methodologies is the inability to align reads with sufficient confidence. To assess read alignment quality in the dataset, overall mapping quality of one million paired end reads was assessed at MQ≧20 and MQ≧30 for each enzyme in both maize and rice and

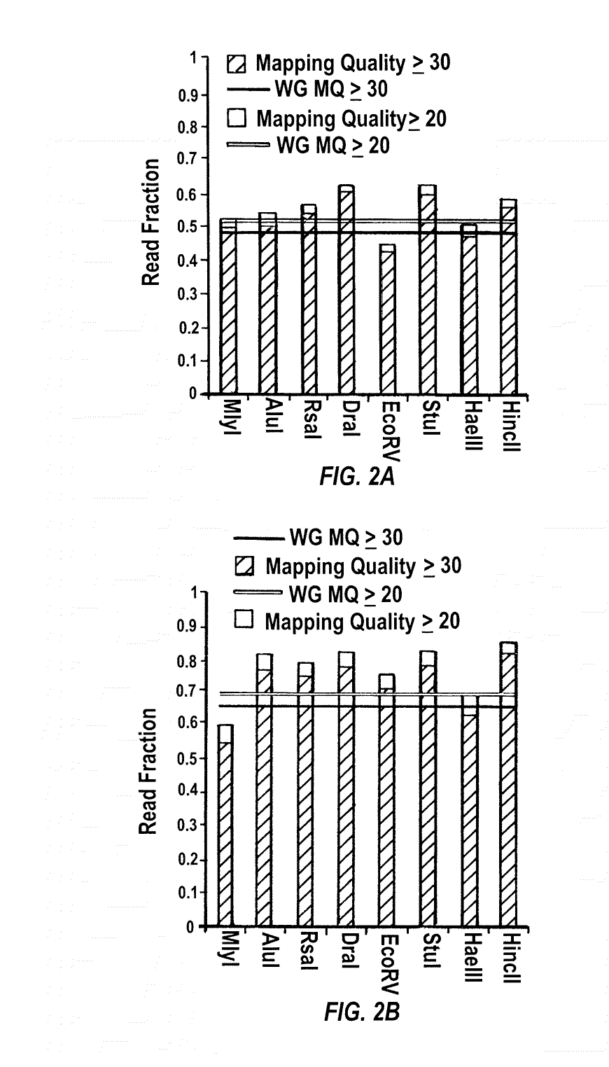

compared to the MQ distribution of whole genome samples consisting of one million paired-end reads truncated to 73 bp. In maize (FIG. 2A) HincII, StuI, and DraI all had MQ scores higher than the whole genome control (0.519≧MQ 20, 0.480≧MQ 30), while RsaI and EcoRV were lower. In rice (FIG. 2B), the majority of enzymes had higher MQ scores than the whole genome dataset (0.697≧MQ 20, 0.668≧MQ 20), except for HaeIII, which was similar in value, and MlyI, which was considerably lower. These studies indicated that enzyme choice influences the proportion of reads that could be confidently aligned to the genome and utilized in downstream experiments.

Trait Mapping

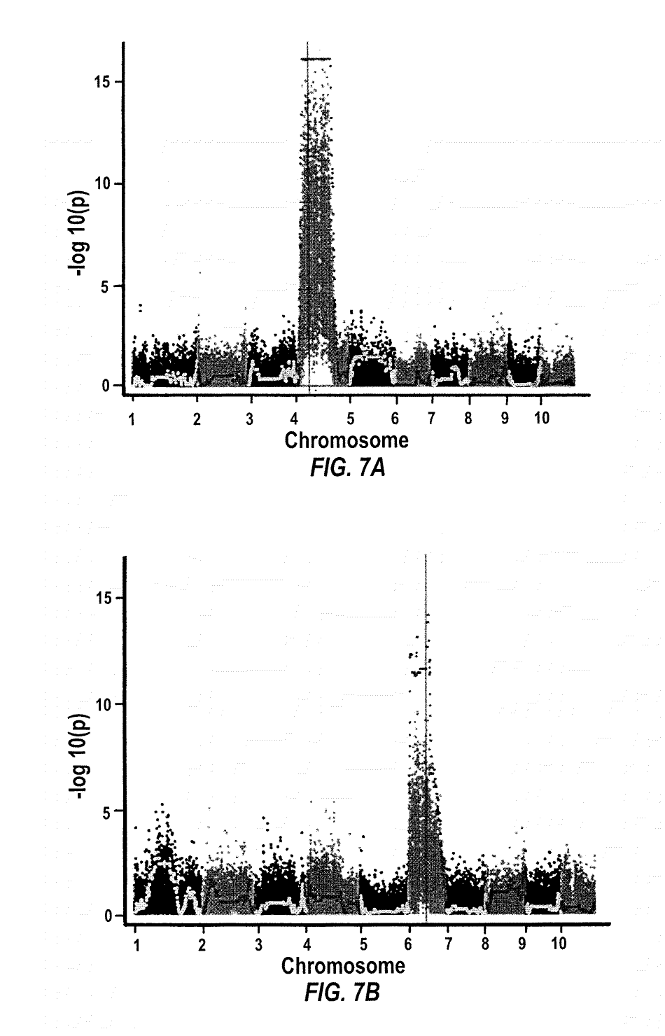

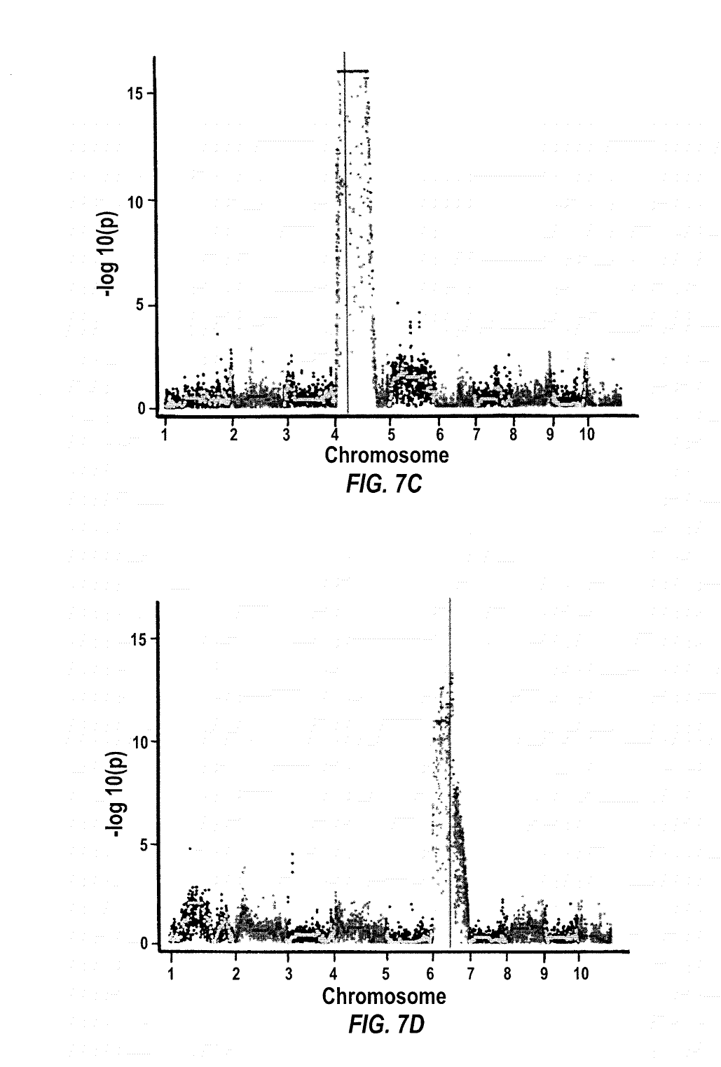

The F2 population segregated for two recessive traits previously mapped in maize: sugary1 (su1) and yellowy1 (y1). The su1 gene maps between Chr4: 41,369,510-41,378,299, and y1 maps between Chr6: 82,017,148-82-020,879. To further validate the variant calling and imputation efficacy of the GBS methodology, these traits were mapped using GBS in the F2 population. A one way ANOVA test on both pre and post imputation datasets of post-filter markers (FIG. 7A-7D) were able to localize causative alleles in the correct regions with p<1E-10.

Variant Calling

Variant filtering is a critical step in identifying informative markers, and special methods are required for GBS datasets. Variants were filtered using a combination of standard and population genomics based criteria. Filtered variants were required to be homozygous, opposite calls in parentals, covered at 2× or greater in at least twenty F2 individuals, MQ and Phred score>30, and r2 correlation≧0.3 with five proximal variants upstream or downstream. A total of 12,499 post-filtration variants were identified in the HincII dataset and 91,894 post-filtration variants were identified in the RsaI dataset.

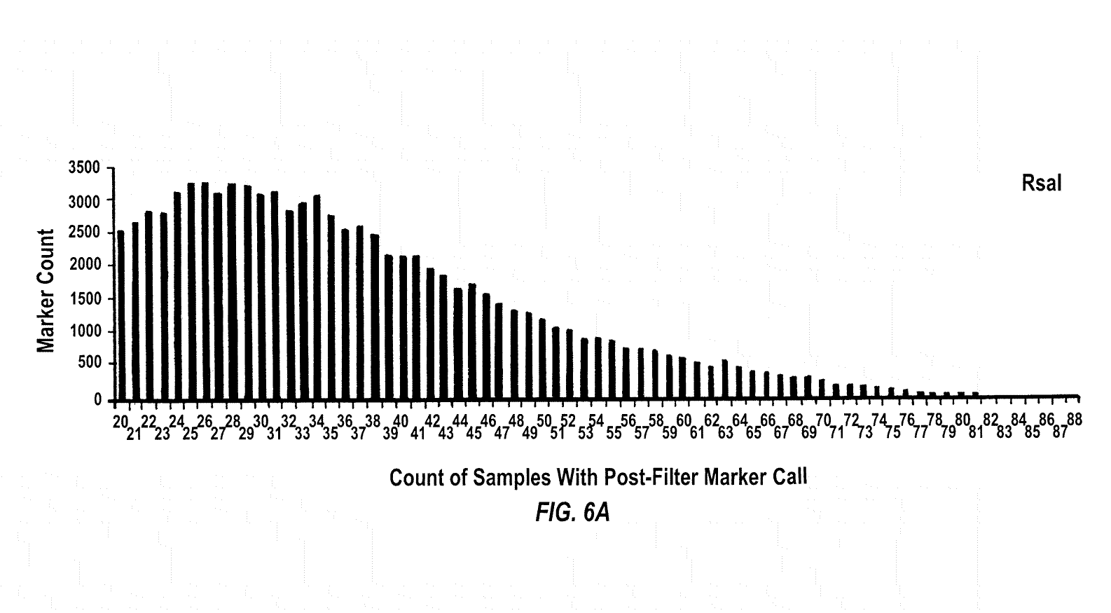

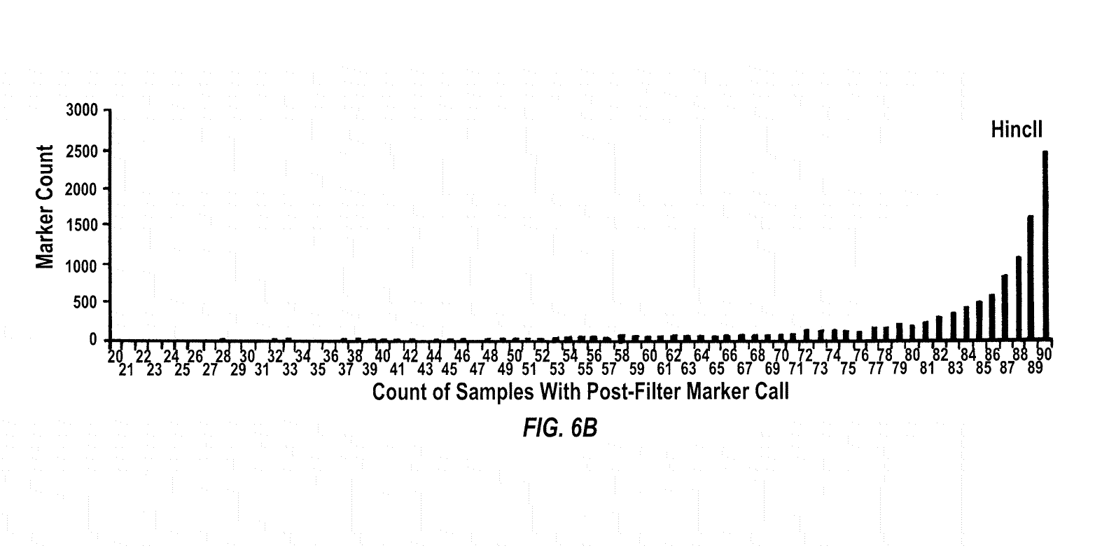

For the RsaI there was a mean per-sample post-filter variant count of 38,439.1±22,133.1 (SD) (FIG. 6A-6B), while HincII had a mean per-sample post-filter variant count of 11,214.7±1093.4 (SD) (FIG. 6A-6B).

Next, parental contribution and recombination breakpoints were determined by imputation of variants by first phasing the final set of variants by parental genotype then applying a least squares algorithm with a sliding window for final genotype calls. F₂ samples typed in both the HincII and RsaI datasets had a concordance of 97.89%±1.00% (SD) on a genomewide, nucleotide level. While large regions with a single genotype were consistent with some minor variation in imputed breakpoint position, the genotype of smaller regions varied between some replicates of samples covered in both the HincII and RsaI datasets. These differences may be due to reduced per-variant sequencing coverage in the RsaI dataset resulting in false homozygous calls in heterozygous regions, or reduced marker density in the HincII dataset resulting in events being missed.

Coverage Simulations

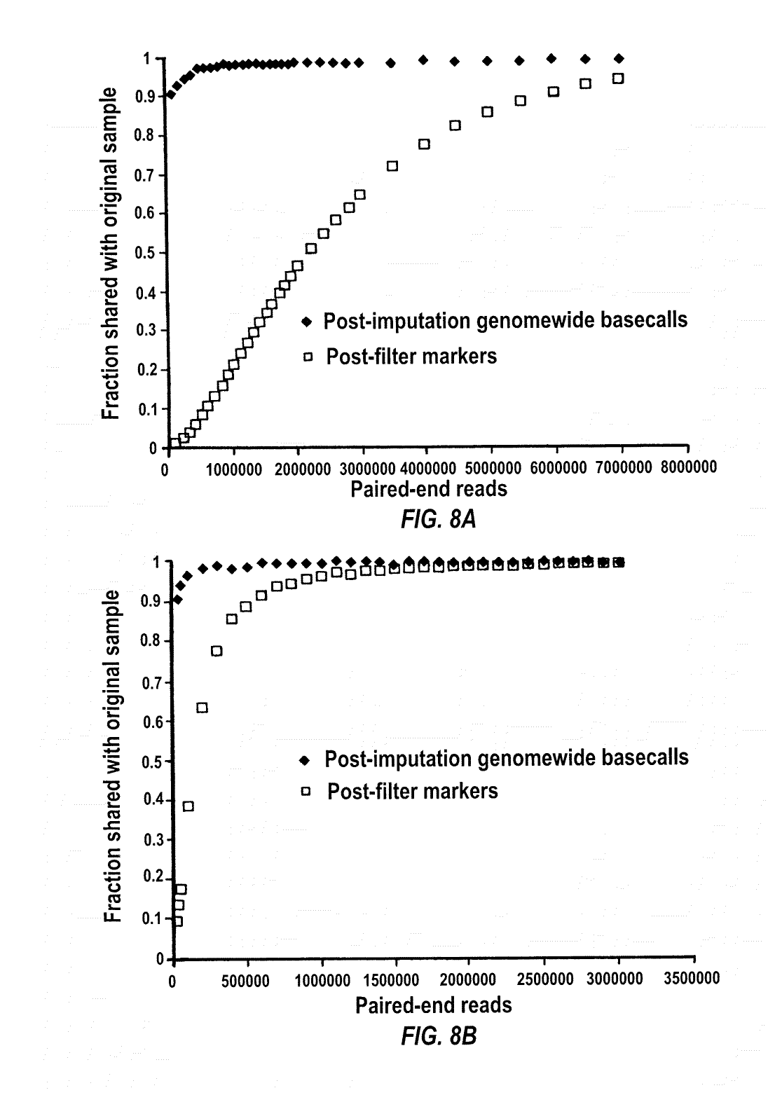

An important consideration in multiplexing for population studies is the per sample depth of coverage. To determine how depth of sequencing coverage affected imputation and marker calling, multiple subsets of randomly selected reads were taken from one RsaI F2 sample (F2-44) and one HincII F2 sample (F2-23). These samples were selected due to their high read-count, which resulted in a near saturation of potential markers (91,584 of 91,894 and 12,154 of 12,499, respectively). Subsets were then realigned against the reference genome, variants were called, and genotypes were imputed. The original RsaI sample contained 15,398,878 reads and 75,593 variant calls. To obtain 90% of the original sample's variant calls, 5,500,000 reads were required (FIG. 8A). The original HincII sample contained 3,698,544 reads and 9728 variant calls. Results indicated that as few as 550,000 reads were required to obtain 90% of the imputed variant calls found in the primary sample (FIG. 8B). The post-imputation genome similarity with the original sample remained above 90% in all read subsets. In both the post-imputation RsaI and HincII datasets, as the number of reads decreased, small recombination events disappeared and possible artifacts began to appear. For RsaI, imputed genome similarity, as measured against the original, high-coverage sample fell beneath 98.0% at 800,000 reads while genome similarity at 100,000 reads fell to only 90.4%. Discordant recombination breakpoints, defined as a pattern of recombination different from that of the primary sample, began to appear at 1.6 million reads. These incongruities were seen as minor segments of miscalled genotypes and discordant localization of recombination breakpoints. For HincII, genome similarity remained at 98% at 100,000 reads and the lowest percent genome similarity was 90.4% at 40,000 reads. Discordant recombination breakpoints began to appear at 500,000 reads.

### Results

Paired Versus Unpaired Sequencing Tags

The modified GBS method produces minimal chimeric reads due to the dA-tailing step. Thus, it is highly suited to paired-end sequencing and associated data analysis. Paired-end reads are generally held to be more likely to align correctly to a genome than single end reads, both due to the increased amount of sequence and the distance between sequences. To evaluate the effect of paired versus single end reads on alignment, the mapping quality of reads was assessed. Mapping quality (MQ) is a measure of confidence in a given read alignment, given the information available in the reference genome. MQ is a Phred scaled value; a MQ of 20 indicates a 1 in 100 chance of misalignment, and a MQ of 30 indicates a 1 in 1000 chance. Reads that map equally well at multiple locations or fail to map at all are given mapping qualities of 0. For many experiments, alignments below a certain mapping quality, usually values of 20, 30 or 40, are filtered out. Sequences were retained as pairs or as “single tags” as in the original GBS protocol (Glaubitz, et al., PloS one, 9(2):e90346 (2014)).

Paired reads are generally held to be more likely to map correctly than unpaired reads (Li, et al., Genome research, 18(11):1851-1858 (2008)).

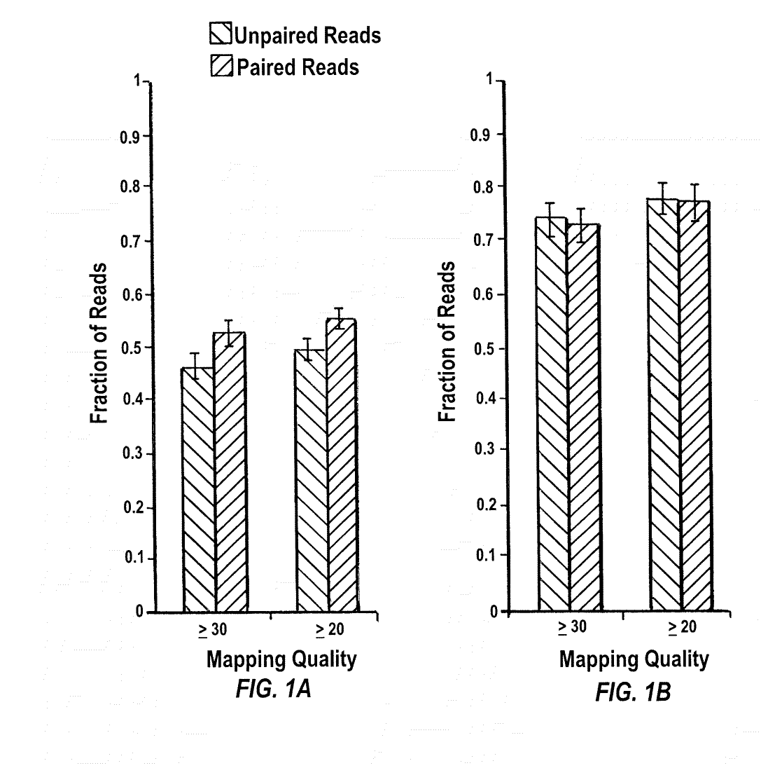

Sequences from each enzyme dataset were aligned as both paired and unpaired reads to the maize and rice reference genomes. The fraction of reads aligning at mapping quality MQ≧20 and MQ≧30 was then determined. In maize (FIG. 1A), a significantly higher fraction of reads in the paired dataset aligned at MQ≧20 (p=0.000, paired t-test) and MQ≧30 (p=0.045, paired t-test). In rice (FIG. 1B), there was no significant difference at MQ≧20 (p=0.077, paired t-test) but a small significant decrease in the fraction of paired reads aligning at MQ≧30 (p=0.045, paired t-test) compared to unpaired reads.

Restriction Motif Presence and Nucleotide Complexity

One initial concern was that the lack of enzyme-specific adaptors might produce more random reads derived from broken DNA fragments. By omitting the end-repair step, digest fragments were enriched, as end-repair both fixes broken ends and adds a phosphate group necessary for adaptor ligation to the 5′ ends of the fragment. The phosphate group is naturally retained on the 5′ with a restriction digest (Pingoud, et al., Nucleic acids research, 29(18):3705-3727 (2001)).

All enzymes save MlyI reliably produced DNA fragments with more than 80% of ends containing the proper restriction motif (Table 1). MlyI, due to its offset cut site, had a restriction motif present in less than half of its reads. Counterintuitive to expectations, this may be beneficial. This is due to how the ILLUMINA® software must calibrate both to identify the cluster boundaries on the flow cell and to assess the quality of nucleotide calls. Proper calibration requires that both the red laser, recognizing G/T and the green laser, recognizing A/C, be sufficiently excited, which requires nucleotide complexity at every cycle in the sequencing run. This is especially important in the early cycles (Krueger, et al., PloS one 2011, 6(1):e16607). As the restriction site for enzymes recognizing palindromic motifs occur at the beginning of a read, this has the potential to severely disrupt a sequencing run.

For most enzymes, namely ones that cut in the center of a palindromic sequence, this means that approximately 20-30% of a run must consist of a “calibration” sample with a random sequence. When whole genome sequence is desired or the sequencing center can arrange to conduct multiple experiments on a single lane, waste is not an issue due to this. When a full lane is desired, custom sequencing protocols may be used that defer cluster coordinate mapping past the motif-containing sequencing cycles (Krueger et al., PloS one, 6(1):e16607 (2011)) or utilize custom sequencing primers that “mask” the restriction site may be used to avoid low-complexity issues. Further, MlyI and other blunt-end restriction enzymes without a cutsite in the center of a palindromic sequence (for example, Type IIS enzymes) do not have this calibration requirement as half or more of the reads will not contain a restriction motif at all.

GBS-Based Population Genomics

The low cost and high multiplexing capacity of the modified GBS protocols indicated that the method would be suitable for population genomics. To test the suitability for trait mapping and population structure analysis, RsaI and HincII restriction digestions were used to create multiplexed GBS libraries from an F₂ population (n=91) derived from a cross between B73 and Country Gentleman (CG) maize inbreds. Eighty-nine RsaI samples and ninety HincII samples were analyzed, with eighty-eight in common to both libraries along with both parental inbreds.

Reads were demultiplexed and aligned to all predicted and covered sites in the B73 reference datasets for RsaI and HincII. No evidence was found of bias due to barcodes, as regression analysis found little correlation between samples sequenced with the same barcodes between HincII and RsaI (slope=0.087, r²=0.071), excluding the fourteen HincII samples that were resequenced. There remains the possibility that certain, specific barcodes will underperform, but these are likely to be only identified through repeated experiments.

As with previous experiments, the results indicated that the highest fraction of covered sites was between 100 and 400 bp. In this range, F2 sites were more concordant with predicted sites covered in the reference B73 datasets as expected. Above 500 bp, the performance of the set of predicted sites covered by the B73 HincII and RsaI datasets was no better than the total set of predicted sites for most F2 samples.

Variant Calling and Filtering

GBS datasets present unique challenges to variant calling and filtering. While traditional metrics like mapping quality and Phred score can be applied, the fixed ends of GBS fragments confound the allelic balance metric and the removal of PCR duplicates by collapsing non-unique reads. Incorporating a low cycle PCR step minimized the latter issue but GBS variant filtering required additional metrics, such as linkage disequilibrium, heterozygosity, and Hardy-Weinberg Equilibrium (HWE). Each of these metrics has circumstantial utility. For instance, linkage disequilibrium analysis requires a reference genome with contigs or scaffolds of sufficient size to compare markers. In wild populations, linkage disequilibrium is highly dependent on population history (Pritchard, et al. American journal of human genetics, 69(1):1-14 (2001)).

HWE is a useful metric for wild populations, but artificial crosses may have issues with segregation distortion or non-random mating. Heterozygosity is applicable to many experiments, but measurements should be corrected for coverage and take into account population history. A final note for any error correction is that variants called from paired-end reads aligning to the same position should be collapsed to a single data point when attempting admixture analysis or trait mapping and should be weighted accordingly. When treating paired-reads as single-end tags, this may cause allelic bias if each tag is treated independently. Many of the error correction tools and concepts have been built into TASSEL, a software package developed for GBS analysis (Glaubitz, et al., PloS one, 9(2):e90346 (2014)).

Trait Mapping in an F2 Population

To validate the modified GBS protocol, two traits, yellowy (y1) and sugary (su1) were mapped in a maize F2 population of ninety-one individuals. Correct locations for each causative allele were identified with both tested enzymes, RsaI and HincII. While data imputation did confer additional significance to association measurements, filtered, unimputed markers were still able to correctly identify the regions containing the causative alleles (FIG. 7A-7D).

RsaI, as was suggested by its marker density profile and overall less complex motif, was able to identify over ninety thousand post-filter markers, compared to just over twelve thousand post-filter markers in the HincII dataset. In addition, each RsaI sample had, on average, three times as many covered markers as per HincII sample. The RsaI and HincII samples both underwent approximately the same amount of sequencing. At first glance, this indicates RsaI was the better enzyme. Higher marker density leads to better resolution of recombination breakpoints. However, what is also noteworthy is the number of samples covered per marker (FIG. 6A-6B). With HincII, markers were covered across almost every sample, while in RsaI each marker was covered in only ˜30% of samples. Further, many RsaI markers even within a few cM to the mapped locations of y1 and su1 did not necessarily show significant association with their respective phenotypes pre-imputation. In HincII, virtually every marker surrounding the previously identified locations for the two mapped traits showed a significant association with phenotypes pre and post imputation. Thus, in scenarios where imputation is not possible, enzymes with a long, complex motif resulting in a more limited set of covered sites may be desirable.

Sequencing Efficiency

Overall sequencing efficiency is a point of interest. GBS libraries prepared using this method lack complexity during the initial few cycles of a sequencing reaction, which much be compensated for as discussed above. They also have a considerably wider size range than a randomly sheared library. Regarding the amount of sequencing that can be expected per lane of the HiSeq 2500, similar results to standard whole genome sequencing on some libraries were obtained. The rice enzyme panel produced just over two hundred million 2×75 bp paired-end reads when run on an ILLUMINA® HiSeq 2500 (rapid mode) lane, which was approximately 33% above what would be expected from a lane of WGS sequencing per ILLUMINA® literature. The maize enzyme panel produced just over one hundred and fifty million reads, or approximately what was expected. The B73×CG F2 populations, both HincII and RsaI, were not run on a single lane however. Both were initially run on 80% of a HiSeq 2500 lane then small amounts of additional resequencing were performed. In the case of RsaI, this was targeted across all samples, whereas for HincII, fourteen specific samples were resequenced. This is likely part of the reason why the coefficient of variation in readcount was much smaller for HincII (0.595) than RsaI (0.928). Variations in sample read count were most likely due to the use of manual pipetting as well as variation in DNA input quantity, as no evidence was found of a correlation between readcounts for samples that shared the same barcodes between datasets (slope=0.087, r2=0.071). Improvements in normalizing DNA input as well as a switch to automatic pipette systems based on later GBS experiments have reduced sample variation considerably.

Enzyme Parameters and Data Quality

The results clearly show that the ability to use a panel of enzymes for GBS has several clear benefits. A major source of data loss in sequencing is the inability to uniquely align reads with sufficient confidence (Li, et al., Genome research, 18(1.1):1851-1858 (2008)). As assessed by mapping quality, certain enzymes, such as DraI, StuI, and HincII produced datasets that were aligned with greater accuracy than others, such as MlyI, HaeIII, and EcoRV in maize (FIGS. 2A-2B). This may reflect a bias against repetitive elements due to motif, or it could be methylation sensitivity limiting digest in repetitive regions.

Enrichment of genic regions was another parameter looked at closely. HincII, RsaI, and AluI in maize produced datasets that contained a considerably greater portion of covered sites in genic regions (FIG. 5A). On the other hand, for MlyI, DraI, EcoRV, and HaeIII in maize as well as all enzymes in rice (FIG. 5B), the proportion of covered sites overlapping genic regions was similar to the genic fraction of total predicted sites. The difference between the two categories appears to be due to methylation sensitivity, which biases enzymes away from cutting the genome in repetitive, heterochromatic regions. The ability to enrich for genic coverage is beneficial in any dataset but is especially beneficial for association studies in populations that have undergone large amounts of recombination. In these studies, a trait may only have associations to markers in the immediate vicinity of the functional variant.

Effect of Genome on Enzyme Selection

Enzyme panels were tested on both B73 maize and Nipponbare rice. While both are critical crop species, their genomes are very dissimilar. The maize genome is large at 2500 Mbp and highly enriched for methylated transposon content. Estimates place the total transposable element content of the B73 genome at above 80% (Tenaillon, et al. Genome biology and evolution, 3:219-229 (2011)). The rice genome is much smaller at approximately 430 Mbp and is much less repetitive at approximately 40% (Goff, et al, Science, 296(5565):92-100(2002)). These parameters resulted in very different experimental outcomes.

The first, and most obvious difference was in the fraction of reads that could be aligned to a genome with high confidence, represented by mapping quality. On average, twenty percent more reads could be aligned with a MQ≧30 in rice than in maize (FIGS. 1A-1B). This is not an unexpected result. What was unexpected was that while paired-end reads conferred a statistically significant improvement in alignment quality over single end reads in maize, they did not do so in rice. In fact, the opposite was observed. Again, this is likely due to the differences in repetitive content between the two genomes. Additional sequence was able to improve the rate of alignment in maize, but in rice, where shorter sequences were more likely to be suitable for a unique alignment, additional sequence just increased the likelihood of sequencing errors reducing the alignment quality.

The second experimental outcome that differed greatly between the two genomes was methylation sensitivity. In maize, HincII, RsaI, and AluI showed significant reductions in G/C content surrounding the restriction motif at sequenced sites versus the predicted G/C content of all possible sites. Further, the fraction of covered reads in genic regions was also greater than predicted by as much as twofold (FIG. 5A). In rice, the proportion of covered sites in genic regions was higher than in maize, the differences between the total predicted and covered datasets tended to be much smaller (FIG. 5B). Further, there was little or no evidence of bias against restriction sites with a potentially methylated motif for any enzyme. This follows the observation that the maize genome contains a much larger proportion of methylated, repetitive content than rice.

The conclusion of the genome comparison, that enzyme choice should take into account the genome of the target organism is not surprising. Utilization of methylation sensitive enzymes avoids repeat content in methylated, repeat rich genomes. Paired-end sequencing in difficult, highly repetitive genomes may produce a considerable increase in useable markers, whereas in much simpler genomes the use of single-end sequencing this may not be an issue. One area that was not directly examined in this study but would likely improve data quality is the use of restriction enzymes that are biased away from repetitive regions by the sequence of their recognition motif. Identifying transposon families or repetitive elements likely to be present in a given genome and selecting an enzyme that does not recognize their sequence may further reduce coverage of unformative regions.

We claim:

1. A method for reduced representation sequencing of a nucleic acid sample comprising:
(a) contacting a first nucleic acid sample with one or more blunt-cutting restriction endonuclease enzyme(s) to form a reaction mix,
(b) incubating the reaction mix under conditions that allow the restriction endonuclease enzyme(s) to cut the nucleic acid to produce a digested nucleic acid sample containing blunt-ended nucleic acid fragments;
(c) ligating the blunt-end nucleic acid fragments in the digested nucleic acid sample to universal sequencing adapters to produce an adapter-ligated digested nucleic acid sample;
(d) performing a complexity reduction on the blunt-ended nucleic acid digestion fragments from the adapter-ligated digested nucleic acid sample to form an enriched nucleic acid sample;
(e) labelling the nucleic acid digestion fragments in the enriched nucleic acid sample with one or more labels to form a labelled, enriched nucleic acid sample; and
(f) sequencing at least a portion of the labelled, enriched nucleic acid sample.

2. The method of stem 1, further comprising
consecutively or simultaneously performing steps (a) through (e) with a second or more nucleic acid sample(s) to obtain a second or more labelled, enriched nucleic acid sample(s); and
combining first and second or more enriched nucleic acid samples before performing step (f).

3. The method of claim 2, further comprising
(g) aligning the sequences obtained in step (f) and determining one or more polymorphisms between the first and second or more nucleic acid samples in the alignment.

4. The method of claim 1 wherein ligating the blunt-end DNA fragments to universal sequencing adapters includes the step of adding adenine to the 3′ end of the DNA fragments.

5. The method of claim 2 wherein the universal sequencing adapters are ILLUMINA® Y-adapters.

6. The method of claim 1, wherein the nucleic acids of step (b), step (c), step (d), step (e), or combinations thereof are bound to a solid phase.

7. The method of claim 6, wherein the solid phase is a multiplicity of magnetic beads.

8. The method of claim 6, further comprising the step of washing the nucleic acids-bound to the solid phase of any of step (b), step (c), step (d), step (e), or combinations thereof.

9. The method of claim 6, wherein one or more of step (b), step (c), step (d) or step (e) are carried out within the same well of a microliter plate.

10. The method of claim 1, wherein the complexity reduction of step (d) comprises fractionation of the adapter-ligated digested nucleic acid sample on the basis of size.

11. The method of claim 10, wherein fractionation of the adapter-ligated digested nucleic acid sample on the basis of size is carried out by selective hybridization of the adapter-ligated digested nucleic acid to magnetic beads.

12. The method of claim 1, wherein the enriched nucleic acid sample comprises DNA fragments with an average length of between 200 and 800 base pairs.

13. The method of claim 1, wherein the first nucleic acid sample includes genomic DNA.

14. The method of claim 13, wherein the first nucleic acid sample includes genomic DNA from more than a single organism.

15. The method of claim 1, wherein labelling the nucleic acid digestion fragments in the enriched nucleic acid sample comprises introducing one or more sequence identifiers to each DNA fragment by polymerase chain reaction.

16. The method of claim 15, wherein the polymerase chain reaction comprises between 2 and 10 cycles, preferably 5 cycles.

17. The method of claim 15, wherein oligonucleotide primers used in the polymerase chain reaction independently comprise a sequence identifier of between four and ten nucleic acid residues.

18. The method of claim 15, wherein the polymerase chain reaction incorporates two unique sequences to each amplified nucleic acid.

19. The method of claim 1, wherein the sequencing of stage (f) comprises paired-end sequencing.

20. The method of claim 13, wherein one or more of the restriction enzymes are selected based on in silico modeling to create a virtual restriction enzyme digest for the genomic nucleic acid sample.

21. A method for sequencing of nucleic acids comprising:
(a) contacting a first nucleic acid sample with one or more blunt-cutting restriction endonuclease enzyme(s) to form a reaction mix,
(b) incubating the reaction mix under conditions that allow the restriction endonuclease enzyme(s) to cut the nucleic acid to produce a digested nucleic acid sample containing blunt-ended nucleic acid fragments;
(c) ligating the blunt-end nucleic acid fragments in the digested nucleic acid sample to universal sequencing adapters to produce an adapter-ligated digested nucleic acid sample;
(d) labelling the nucleic acid digestion fragments in the adapter-ligated digested nucleic acid sample with one or more labels to form a labelled nucleic acid sample; and
(e) sequencing at least a portion of the labelled nucleic acid sample.

22. A kit for conducting the method of claim 6, comprising
(a) one or more blunt-cutting restriction endonuclease enzyme(s);
(b) universal sequencing adapters;
(c) SPRI beads; and
(d) instructions for carrying out the method of claim 5.


SEQUENCE LISTING (16 sequences)

SEQ ID NO: 1 - DNA, 33 bp, Artificial Sequence
  Synthetic Oligonucleotide
gatcggaagagcacacgtctgaactccagtcac

SEQ ID NO: 2 - DNA, 33 bp, Artificial Sequence
  Synthetic Oligonucleotide
acactctttccctacacgacgctcttccgatct

SEQ ID NO: 3 - DNA, 62 bp, Artificial Sequence
  Synthetic Oligonucleotide
caagcagaagacggcatacgagatnnnngtgactggagttcagacgtgtgctcttccgatct

SEQ ID NO: 4 - DNA, 63 bp, Artificial Sequence
  Synthetic Oligonucleotide
caagcagaagacggcatacgagatnnnnngtgactggagttcagacgtgtgctcttccgatct

SEQ ID NO: 5 - DNA, 64 bp, Artificial Sequence
  Synthetic Oligonucleotide
caagcagaagacggcatacgagatnnnnnngtgactggagttcagacgtgtgctcttccgatct

SEQ ID NO: 6 - DNA, 65 bp, Artificial Sequence
  Synthetic Oligonucleotide
caagcagaagacggcatacgagatnnnnnnngtgactggagttcagacgtgtgctcttccgatct

SEQ ID NO: 7 - DNA, 66 bp, Artificial Sequence
  Synthetic Oligonucleotide
caagcagaagacggcatacgagatnnnnnnnngtgactggagttcagacgtgtgctcttccgatct

SEQ ID NO: 8 - DNA, 67 bp, Artificial Sequence
  Synthetic Oligonucleotide
caagcagaagacggcatacgagatnnnnnnnnngtgactggagttcagacgtgtgctcttccgatct

SEQ ID NO: 9 - DNA, 68 bp, Artificial Sequence
  Synthetic Oligonucleotide
caagcagaagacggcatacgagatnnnnnnnnnngtgactggagttcagacgtgtgctcttccgatct

SEQ ID NO: 10 - DNA, 66 bp, Artificial Sequence
  Synthetic Oligonucleotide
aatgatacggcgaccaccgagatctacacnnnnacactctttccctacacgacgctcttccgatct

SEQ ID NO: 11 - DNA, 67 bp, Artificial Sequence
  Synthetic Oligonucleotide
aatgatacggcgaccaccgagatctacacnnnnnacactctttccctacacgacgctcttccgatct

SEQ ID NO: 12 - DNA, 68 bp, Artificial Sequence
  Synthetic Oligonucleotide
aatgatacggcgaccaccgagatctacacnnnnnnacactctttccctacacgacgctcttccgatct

SEQ ID NO: 13 - DNA, 69 bp, Artificial Sequence
  Synthetic Oligonucleotide
aatgatacggcgaccaccgagatctacacnnnnnnnacactctttccctacacgacgctcttccgatct

SEQ ID NO: 14 - DNA, 70 bp, Artificial Sequence
  Synthetic Oligonucleotide
aatgatacggcgaccaccgagatctacacnnnnnnnnacactctttccctacacgacgctcttccgatct

SEQ ID NO: 15 - DNA, 71 bp, Artificial Sequence
  Synthetic Oligonucleotide
aatgatacggcgaccaccgagatctacacnnnnnnnnnacactctttccctacacgacgctcttccgatct

SEQ ID NO: 16 - DNA, 72 bp, Artificial Sequence
  Synthetic Oligonucleotide
aatgatacggcgaccaccgagatctacacnnnnnnnnnnacactctttccctacacgacgctcttccgatct